(19) United States
(12) Patent Application Publication
(10) Pub. No.: US 2019/0256930 A1
(43) Pub. Date: Aug. 22, 2019

(54) BIOMARKERS FOR DETERMINING RESPONSIVENESS TO LSD1 INHIBITORS

(71) Applicant: ORYZON GENOMICS, S.A., Madrid (ES)

(72) Inventors: Maria Isabel ARÉVALO SÁNCHEZ, Cornella de Llobregat (ES); Serena LUNARDI, Cornella de Llobregat (ES); Tamara MAES, Cornella de Llobregat (ES); Cristina MASCARO CRUSAT, Cornella de Llobregat (ES)

(21) Appl. No.: 16/346,915

(22) PCT Filed: Nov. 2, 2017
(86) PCT No.: PCT/EP2017/078084

§ 371 Date: May 2, 2019

(30) Foreign Application Priority Data
Nov. 3, 2016 (EP) 16382506.0
Dec. 2, 2016 (EP) 16382588.8

(51) Int. Cl. C12Q 1/6886; A61P 35/02; A61K 31/135

(52) U.S. Cl. CPC C12Q 1/6886; A61P 35/02; C12Q 2600/106; C12Q 2600/158; C12Q 2600/118; A61K 31/135

(57) ABSTRACT

The present invention relates to methods for monitoring the response to treatment with an LSD1 inhibitor in a subject suffering from leukemia. The present invention also provides methods for the identification of a responding subject to treatment with an LSD1 inhibitor. Also methods of determining whether a proliferative diseased cell is responsive to treatment with an LSD1 inhibitor are provided. The methods comprise determining the level of one or more of markers in a sample, wherein an increased level of one or more of said markers compared to a control indicates responsiveness to the LSD1 inhibitor. Methods of treatment of patients with the LSD1 inhibitor, wherein the patients are identified in accordance with the present invention to be responders are also subject of the present invention. LSD1 inhibitors for use in the treatment of this patient group are provided.

The present invention relates to methods for monitoring the response to treatment with an LSD1 inhibitor in a subject suffering from leukemia. The present invention also provides methods for the identification of a responding subject to treatment with an LSD1 inhibitor. Also methods of determining whether a proliferative diseased cell is responsive to treatment with an LSD1 inhibitor are provided. The methods comprise determining the level of one or more of the markers S100A12, VCAN, ITGAM, LY96, ANXA2, CD86, GPR65, CRISP9, LYZ and VIM, in a sample, wherein an increased level of one or more of the markers S100A12, VCAN, ITGAM, LY96, ANXA2, CD86, GPR65, CRISP9, LYZ and VIM compared to a control indicates responsiveness to the LSD1 inhibitor. Methods of treatment of patients with the LSD1 inhibitor, wherein the patients are identified in accordance with the present invention to be responders are also subject of the present invention. LSD1 inhibitors for use in the treatment of this patient group are provided.

Aberrant gene expression in affected tissue as compared to normal tissue is a common characteristic of many human diseases. This is true for cancer and many neurological diseases which are characterized by changes in gene expression patterns. Gene expression patterns are controlled at multiple levels in the cell. Control of gene expression can occur through modifications of DNA: DNA promoter methylation is associated with suppression of gene expression. Another class of modifications involve histones, which are proteins, present in the nucleus of eukaryotic cells, that organize DNA strands into nucleosomes by forming molecular complexes around which the DNA winds. Histones play a critical role in modulating chromatin structure and DNA accessibility for replication, repair, and transcription. The covalent modification of histones is closely associated with regulation of gene transcription. Chromatin modifications have been suggested to represent an epigenetic code that is dynamically ‘written’ and ‘erased’ by specialized proteins, and ‘read’ or interpreted by proteins that translate the code into gene expression changes. A number of histone modifications have been discovered including histone acetylation, histone lysine methylation, histone arginine methylation, histone ubiquinylation, and histone sumoylation.

A group of enzymes known as histone lysine methyl transferases and histone lysine demethylases are involved in histone lysine modifications. One particular human histone lysine demethylase enzyme called Lysine Specific Demethylase-1 (LSD1) (Shi et al. (2004) Cell 119:941) has been reported to be involved in this crucial histone modification. LSD1 has a fair degree of structural similarity, and amino acid identity/homology to polyamine oxidases and monoamine oxidases, all of which (i.e., MAO-A, MAO-B and LSD1) are flavin dependent amine oxidases which catalyze the oxidation of nitrogen-hydrogen bonds and/or nitrogen carbon bonds.

LSD1 has been recognized as an interesting target for the development of new drugs to treat cancer, neurological diseases and other conditions, and a number of LSD1 inhibitors are currently under preclinical or clinical development for use in human therapy.

Finding pharmacodynamic (PD) biomarkers which indicate that a drug is active can be valuable for use during clinical trials or in clinical practice. PD biomarkers can be used to monitor target engagement, i.e. to see if the drug is inhibiting the target against which the drug is designed to act in a subject receiving such drug. They can also be used to monitor the response of those patients receiving the drug. If the biomarker indicates that the patient is not responding appropriately to the drug treatment, then the dosage administered can be increased, reduced or treatment can be discontinued. Biomarkers can also be used to identify particular groups of patients that would benefit, or that would benefit the most, from receiving the drug treatment.

The technical problem underlying the present invention is the provision of means and methods to monitor the response to treatment with an LSD1 inhibitor in subjects suffering from leukemia and to identify subjects suffering from leukemia that respond to an LSD1 inhibitor.

The technical problem is solved by provision of the embodiments characterized in the claims.

Accordingly, the present invention relates to a method for monitoring the response to treatment with an LSD1 inhibitor in a subject suffering from leukemia, said method comprising determining the level of one or more of the markers S100A12, VCAN, ITGAM, LY96, ANXA2, CD86, GPR65, CRISP9, LYZ and VIM, in a sample from said subject, wherein an increased level of one or more of the markers S100A12, VCAN, ITGAM, LY96, ANXA2, CD86, GPR65, CRISP9, LYZ and VIM compared to a control is indicative for response to treatment.

In a further aspect, the present invention relates to a method for the identification of a responding subject to treatment with an LSD1 inhibitor, said method comprising determining the level of one or more of the markers S100A12, VCAN, ITGAM, LY96, ANXA2, CD86, GPR65, CRISP9, LYZ and VIM in a sample from a subject suffering from leukemia, wherein an increased level of one or more of the markers S100A12, VCAN, ITGAM, LY96, ANXA2, CD86, GPR65, CRISP9, LYZ and VIM compared to a control is indicative for a responding subject.

In a related aspect, the present invention relates to a method of determining whether a proliferative diseased cell is responsive to treatment with an LSD1 inhibitor, said method comprising determining the level of one or more of the markers S100A12, VCAN, ITGAM, LY96, ANXA2, CD86, GPR65, CRISP9, LYZ and VIM in a sample from a subject suffering from leukemia, wherein an increased level of one or more of the markers S100A12, VCAN, ITGAM, LY96, ANXA2, CD86, GPR65, CRISP9, LYZ and VIM compared to a control is indicative for a responsive proliferative diseased cell.

As documented herein below and in the appended examples, levels of biomarkers in subjects suffering from leukemia, especially acute myeloid leukemia (AML), were determined during the course of treatment with an LSD1 inhibitor. The level was correlated to response to the LSD1 inhibitor (increase in blast differentiation and/or a decrease in blast cells). Thereby, a panel of biomarkers was identified whose increased expression level correlated with the response to the LSD1 inhibitor. These biomarkers were therefore demonstrated herein as being useful for monitoring a response to LSD1 inhibitor in leukemia patients. They can also serve to identify responders to LSD1 inhibitors.

It was demonstrated herein that not all potential biomarkers that are differentially regulated during LSD1 inhibitor treatment are useful for monitoring a response to LSD1 inhibitors in leukemia patients. For example, patient 9 showed a response to LSD1 inhibitor treatment (in this patient, blast differentiation and decrease in blasts was observed), and a decrease in the level of CTSG. However, the level of CTSG was increased in patients 1 and 2, that also responded to LSD1 inhibitor treatment. Thus, the level of CTSG is not consistently increased or decreased in responding subjects. Likewise, the level of CAMSAP2 was decreased in responding patient 9 and increased in responding patients 1, and 2, and 4. Therefore, also the level of CAMSAP2 is not consistently increased or decreased in responding subjects. Thus, CTSG and CAMSAP2 were determined not to be useful for monitoring a response to LSD1 inhibitor treatment.

By contrast, levels of the markers S100A12, VCAN, ITGAM, LY96, ANXA2, CD86, GPR65, CRISP9, LYZ, and VIM were consistently increased in leukemia patients that responded to treatment with an LSD1 inhibitor. This increased expression level of these biomarkers was particularly consistent and pronounced in AML patients of AML subtype M4 and M5 (see patients 1, 2 and 9). Thus, biomarkers S100A12, VCAN, ITGAM, LY96, ANXA2, CD86, GPR65, CRISP9, LYZ, and/or VIM are useful to monitor a response to treatment with an LSD1 inhibitor and/or to identify responders. Their use may be particularly advantageous in the patient group of AML subtype M4 and M5. The highest increase was seen for biomarkers S100A12, VCAN, and LY96, particularly in samples from responding patients of the AML M4/M5 subtypes.

Additionally, the expression levels of biomarkers of the invention correlate with the variation of blast cells in bone marrow, particularly in M4/M5 subtypes, further supporting the utility of these marker genes in monitoring response to LSD1 inhibitor treatment in easily accessible samples such as peripheral blood. In particular, the expression levels of Ly96 and ITGAM correlate with the variation of blast cells in bone marrow particularly in M4/M5 subtypes.

In the herein provided experiments peripheral blood samples obtained from the patients have been used. While the present invention is not limited to this type of sample, the use of blood samples is particularly advantageous. Blood extractions are easy to perform and can be performed more frequently than biopsies or bone marrow sampling, and leukemia patients are subject to frequent hemogram analysis. Therefore, a monitoring method that can be used to assess the response to (treatment with) an LSD1 inhibitor in blood samples as described herein is highly desirable.

Unexpectedly, the herein provided biomarkers are not only useful to monitor response to an LSD1 inhibitor or identify responders to treatment with an LSD1 inhibitor. It was shown herein that the biomarkers can also be used to predict whether a subject is at risk of developing a differentiation syndrome (DS). The differentiation syndrome (DS) is a relatively common and potentially severe complication seen in AML patients treated with differentiating agents. LSD1 inhibitors have been shown to induce differentiation of leukemic blast cells. The differentiation of a vast number of leukemic blasts may lead to cellular migration, endothelial activation, and release of interleukins and vascular factors responsible for tissue damage, finally developing in a syndrome characterized by unexplained fever, acute respiratory distress with interstitial pulmonary infiltrates, and/or a vascular capillary leak leading to acute renal failure. In fact, patients 1 and 9 herein that responded well to LSD1 inhibitor treatment developed a differentiation syndrome in the course of the treatment. As demonstrated herein, biomarkers S100A12 and VCAN showed an exacerbated (18 to 550-fold) up-regulation in these patients. Importantly, this up-regulation could be observed up to 2 weeks prior to the clinical diagnosis of the differentiation syndrome. Thus, measuring the increase of S100A12 and VCAN is a useful tool to early monitor the risk of developing a differentiation syndrome in leukemia patients receiving treatment with an LSD1 inhibitor (e.g. ORY-1001), particularly in AML M4/M5 subtypes.

As explained above and shown in the appended examples, S100A12, VCAN, ITGAM, LY96, ANXA2, CD86, GPR65, CRISP9, LYZ, and VIM are highly useful biomarkers for monitoring a response to an LSD1 inhibitor or for identifiying responders. Therefore, S100A12, VCAN, ITGAM, LY96, ANXA2, CD86, GPR65, CRISP9, LYZ, and VIM can be used advantageously in accordance with the present invention. Subsets of these markers may be particularly advantageously used for specific applications, e.g. for discriminating best responders and worse responders and/or for assessing the risk of developing a differentiation syndrome among those subjects receiving treatment with an LSD1 inhibitor. Based on an overall assessment of the experimental data provided herein, S100A12, VCAN, ITGAM, LY96, ANXA2, CD86, GPR65, CRISP9, and/or LYZ, would be preferred biomarkers for use in the present invention. A more limited panel of one or more of biomarkers S100A12, VCAN, ITGAM, LY96, ANXA2, and CD86, would be particularly preferred for use in the present invention.

The terms “marker”/“markers” and “biomarker”/“biomarkers” are used interchangeably herein.

As mentioned above, the present invention relates to a method for monitoring the response to treatment with an LSD1 inhibitor in a subject suffering from leukemia, said method comprising determining the level of one or more of the markers S100A12, VCAN, ITGAM, LY96, ANXA2, CD86, GPR65, CRISP9, LYZ and VIM, in a sample from said subject, wherein an increased level of one or more of the markers S100A12, VCAN, ITGAM, LY96, ANXA2, CD86, GPR65, CRISP9, LYZ and VIM compared to a control is indicative for response to treatment.

It is understood that the response of a subject to an LSD1 inhibitor/to treatment with an LSD1 inhibitor is monitored.

The monitoring method of the invention relates therefore in other words to a method for monitoring the response of a subject suffering from leukemia to treatment with an LSD1 inhibitor, said method comprising determining the level of one or more of the markers S100A12, VCAN, ITGAM, LY96, ANXA2, CD86, GPR65, CRISP9, LYZ and VIM, in a sample from said subject, wherein an increased level of one or more of the markers S100A12, VCAN, ITGAM, LY96, ANXA2, CD86, GPR65, CRISP9, LYZ and VIM compared to a control is indicative for response of said subject to treatment. In yet other words, the present invention relates in an aspect to a method for monitoring the response to treatment with an LSD1 inhibitor, said method comprising determining the level of one or more of the markers S100A12, VCAN, ITGAM, LY96, ANXA2, CD86, GPR65, CRISP9, LYZ and VIM, in a sample from a subject suffering from leukemia, wherein an increased level of one or more of the markers S100A12, VCAN, ITGAM, LY96, ANXA2, CD86, GPR65, CRISP9, LYZ and VIM compared to a control is indicative for response of said subject to treatment.

The term “treatment” as used in the present invention relates in its broadest sense to the administration of an LSD1 inhibitor to a subject suffering from leukemia. In a more simplified form, the terms “response to treatment with an LSD1 inhibitor in a subject suffering from leukemia” or “treatment with an LSD1 inhibitor in a subject suffering from leukemia” and the like can be phrased “response to an LSD1 inhibitor in a subject suffering from leukemia” or “an LSD1 inhibitor in a subject suffering from leukemia” and the like.

Thus, the present invention relates in other words in this sense to a method for monitoring the response to an LSD1 inhibitor in a subject suffering from leukemia, said method comprising determining the level of one or more of the markers S100A12, VCAN, ITGAM, LY96, ANXA2, CD86, GPR65, CRISP9, LYZ and VIM, in a sample from said subject, wherein an increased level of one or more of the markers S100A12, VCAN, ITGAM, LY96, ANXA2, CD86, GPR65, CRISP9, LYZ and VIM compared to a control is indicative for response to said LSD1 inhibitor. Likewise, the monitoring method of the invention relates in one aspect to a method for monitoring the response of a subject suffering from leukemia to an LSD1 inhibitor, said method comprising determining the level of one or more of the markers S100A12, VCAN, ITGAM, LY96, ANXA2, CD86, GPR65, CRISP9, LYZ and VIM, in a sample from said subject, wherein an increased level of one or more of the markers S100A12, VCAN, ITGAM, LY96, ANXA2, CD86, GPR65, CRISP9, LYZ and VIM compared to a control is indicative for response of said subject to said LSD1 inhibitor. Likewise, the present invention relates in an aspect to a method for monitoring the response to an LSD1 inhibitor, said method comprising determining the level of one or more of the markers S100A12, VCAN, ITGAM, LY96, ANXA2, CD86, GPR65, CRISP9, LYZ and VIM, in a sample from a subject suffering from leukemia, wherein an increased level of one or more of the markers S100A12, VCAN, ITGAM, LY96, ANXA2, CD86, GPR65, CRISP9, LYZ and VIM compared to a control is indicative for response to said LSD1 inhibitor.

The method can comprise a step of comparing the level of one or more of the markers S100A12, VCAN, ITGAM, LY96, ANXA2, CD86, GPR65, CRISP9, LYZ and VIM with a control.

The present invention relates in one aspect accordingly to a method for monitoring the response to treatment with an LSD1 inhibitor in a subject suffering from leukemia, said method comprising
(a) determining the level of one or more of the markers S100A12, VCAN, ITGAM, LY96, ANXA2, CD86, GPR65, CRISP9, LYZ and VIM, in a sample from said subject,
(b) comparing the level of one or more of the markers S100A12, VCAN, ITGAM, LY96, ANXA2, CD86, GPR65, CRISP9, LYZ and VIM determined in a) with a control.

According to said method, an increased level of one or more of the markers S100A12, VCAN, ITGAM, LY96, ANXA2, CD86, GPR65, CRISP9, LYZ and VIM determined in a) compared to a control indicates a response to the treatment with an LSD1 inhibitor.

The monitoring method of the invention relates therefore in other words to a method for monitoring the response of a subject suffering from leukemia to treatment with an LSD1 inhibitor, said method comprising
(a) determining the level of one or more of the markers S100A12, VCAN, ITGAM, LY96, ANXA2, CD86, GPR65, CRISP9, LYZ and VIM, in a sample from said subject,
(b) comparing the level of one or more of the markers S100A12, VCAN, ITGAM, LY96, ANXA2, CD86, GPR65, CRISP9, LYZ and VIM determined in a) with a control.

According to said method, an increased level of one or more of the markers S100A12, VCAN, ITGAM, LY96, ANXA2, CD86, GPR65, CRISP9, LYZ and VIM determined in a) compared to a control indicates a response of said subject to the treatment of leukemia with an LSD1 inhibitor.

In yet other words, the present invention relates in an aspect to a method for monitoring the response to treatment with an LSD1 inhibitor, said method comprising
(a) determining the level of one or more of the markers S100A12, VCAN, ITGAM, LY96, ANXA2, CD86, GPR65, CRISP9, LYZ and VIM, in a sample from a subject suffering from leukemia,
(b) comparing the level of one or more of the markers S100A12, VCAN, ITGAM, LY96, ANXA2, CD86, GPR65, CRISP9, LYZ and VIM determined in a) with a control.

According to said method, an increased level of one or more of the markers S100A12, VCAN, ITGAM, LY96, ANXA2, CD86, GPR65, CRISP9, LYZ and VIM compared to a control is indicative for response to treatment.

The term “monitoring the response” as used herein can include or can be an assessment of the response.

The monitoring method of the invention relates therefore in other words in one aspect to a method for assessing the response to treatment with an LSD1 inhibitor in a subject suffering from leukemia, said method comprising assessing the level of one or more of the markers S100A12, VCAN, ITGAM, LY96, ANXA2, CD86, GPR65, CRISP9, LYZ and VIM, in a sample from said subject, wherein an increased level of one or more of the markers S100A12, VCAN, ITGAM, LY96, ANXA2, CD86, GPR65, CRISP9, LYZ and VIM compared to a control is indicative for response to treatment. In another aspect, the present invention relates to a method for assessing the response of a subject suffering from leukemia to treatment with an LSD1 inhibitor, said method comprising assessing the level of one or more of the markers S100A12, VCAN, ITGAM, LY96, ANXA2, CD86, GPR65, CRISP9, LYZ and VIM, in a sample from said subject, wherein an increased level of one or more of the markers S100A12, VCAN, ITGAM, LY96, ANXA2, CD86, GPR65, CRISP9, LYZ and VIM compared to a control is indicative for response of said subject to treatment. In yet other words, the present invention relates in an aspect to a method for assessing the response to treatment with an LSD1 inhibitor, said method comprising assessing the level of one or more of the markers S100A12, VCAN, ITGAM, LY96, AN , CD86, GPR65, CRISP9, LYZ and VIM, in a sample from a subject suffering from leukemia, wherein an increased level of one or more of the markers S100A12, VCAN, ITGAM, LY96, ANXA2, CD86, GPR65, CRISP9, LYZ and VIM compared to a control is indicative for response of said subject to treatment.

It is understood that the term “treatment with an LSD1 inhibitor” as used herein can be a “therapy comprising an LSD1 inhibitor”.

The term “response (to treatment with an LSD1 inhibitor”) as used herein can include or can be “efficacy (of treatment with an LSD1 inhibitor)”.

The monitoring method of the invention relates therefore in other words to a method for monitoring the efficacy of treatment with an LSD1 inhibitor in a subject suffering from leukemia, said method comprising monitoring the level of one or more of the markers S100A12, VCAN, ITGAM, LY96, ANXA2, CD86, GPR65, CRISP9, LYZ and VIM, in a sample from said subject, wherein an increased level of one or more of the markers S100A12, VCAN, ITGAM, LY96, ANXA2, CD86, GPR65, CRISP9, LYZ and VIM compared to a control is indicative for efficacy of the treatment. In yet other words, the present invention relates in an aspect to a method for monitoring the efficacy of treatment with an LSD1 inhibitor, said method comprising monitoring the level of one or more of the markers S100A12, VCAN, ITGAM, LY96, ANXA2, CD86, GPR65, CRISP9, LYZ and VIM, in a sample from a subject suffering from leukemia, wherein an increased level of one or more of the markers S100A12, VCAN, ITGAM, LY96, ANXA2, CD86, GPR65, CRISP9, LYZ and VIM compared to a control is indicative for efficacy of said treatment.

As mentioned above, the present invention relates to a method for monitoring the response to treatment with an LSD1 inhibitor in a subject suffering from leukemia, said method comprising determining the level of one or more of the markers S100A12, VCAN, ITGAM, LY96, ANXA2, CD86, GPR65, CRISP9, LYZ and VIM, in a sample from said subject, wherein an increased level of one or more of the markers S100A12, VCAN, ITGAM, LY96, ANXA2, CD86, GPR65, CRISP9, LYZ and VIM compared to a control is indicative for response to treatment.

It is understood that the term “indicative” as used herein refers to the fact that an increase in the level of one or more of the biomarkers disclosed herein reflects the response to (treatment with) an LSD1 inhibitor. Accordingly, the methods of the invention can also be phrased in a more assertive way without deferring from the gist of the invention, e.g. by stating that if the level of one or more of the markers S100A12, VCAN, ITGAM, LY96, ANXA2, CD86, GPR65, CRISP9, LYZ and VIM is increased compared to a control, the subject is identified as responsive to (treatment with) an LSD1 inhibitor.

For example, the present invention can accordingly relate in one aspect to a method for monitoring the response to treatment with an LSD1 inhibitor in a subject suffering from leukemia, said method comprising determining the level of one or more of the markers S100A12, VCAN, ITGAM, LY96, ANXA2, CD86, GPR65, CRISP9, LYZ and VIM, in a sample from said subject, wherein if the level of one or more of the markers S100A12, VCAN, ITGAM, LY96, ANXA2, CD86, GPR65, CRISP9, LYZ and VIM is increased compared to a control, the subject is responsive to treatment with an LSD1 inhibitor. The monitoring method of the invention can likewise relate to a method for monitoring the response of a subject suffering from leukemia to treatment with an LSD1 inhibitor, said method comprising determining the level of one or more of the markers S100A12, VCAN, ITGAM, LY96, ANXA2, CD86, GPR65, CRISP9, LYZ and VIM, in a sample from said subject, wherein if the level of one or more of the markers S100A12, VCAN, ITGAM, LY96, ANXA2, CD86, GPR65, CRISP9, LYZ and VIM is increased compared to a control, the subject is responsive to treatment with an LSD1 inhibitor. In yet other words, the present invention relates in an aspect to a method for monitoring the response to treatment with an LSD1 inhibitor, said method comprising determining the level of one or more of the markers S100A12, VCAN, ITGAM, LY96, ANXA2, CD86, GPR65, CRISP9, LYZ and VIM, in a sample from a subject suffering from leukemia, wherein if the level of one or more of the markers S100A12, VCAN, ITGAM, LY96, ANXA2, CD86, GPR65, CRISP9, LYZ and VIM is increased compared to a control, the subject is responsive to treatment with an LSD1 inhibitor.

The methods of the invention serve to monitor the response to (treatment with) an LSD1 inhibitor. They thus can be used to identify responding subjects and/or to identify a responding proliferative diseased cell.

Thus, the present invention relates in a related aspect to a method for the identification of a responding subject to treatment with an LSD1 inhibitor, said method comprising determining the level of one or more of the markers S100A12, VCAN, ITGAM, LY96, ANXA2, CD86, GPR65, CRISP9, LYZ and VIM in a sample from a subject suffering from leukemia, wherein an increased level of one or more of the markers S100A12, VCAN, ITGAM, LY96, ANXA2, CD86, GPR65, CRISP9, LYZ and VIM compared to a control is indicative for a responding subject.

In other words, the present invention relates in a one aspect to a method for the identification of a responding subject to treatment with an LSD1 inhibitor, said method comprising determining the level of one or more of the markers S100A12, VCAN, ITGAM, LY96, ANXA2, CD86, GPR65, CRISP9, LYZ and VIM in a sample from a subject suffering from leukemia, wherein if the level of one or more of the markers S100A12, VCAN, ITGAM, LY96, ANXA2, CD86, GPR65, CRISP9, LYZ and VIM is increased compared to a control, the subject is responsive to treatment with an LSD1 inhibitor.

In a related aspect, the present invention relates to a method of determining whether a proliferative diseased cell is responsive to treatment with an LSD1 inhibitor, said method comprising determining the level of one or more of the markers S100A12, VCAN, ITGAM, LY96, ANXA2, CD86, GPR65, CRISP9, LYZ and VIM in a sample from a subject suffering from leukemia, wherein an increased level of one or more of the markers S100A12, VCAN, ITGAM, LY96, ANXA2, CD86, GPR65, CRISP9, LYZ and VIM compared to a control is indicative for a responsive proliferative diseased cell. In other words, the present invention relates to a method of determining whether a proliferative diseased cell is responsive to treatment with an LSD1 inhibitor, said method comprising determining the level of one or more of the markers S100A12, VCAN, ITGAM, LY96, ANXA2, CD86, GPR65, CRISP9, LYZ and VIM in a sample from a subject suffering from leukemia, wherein if the level of one or more of the markers S100A12, VCAN, ITGAM, LY96, ANXA2, CD86, GPR65, CRISP9, LYZ and VIM is increased compared to a control, the proliferative diseased cell is responsive to treatment with an LSD1 inhibitor.]

As mentioned above, the term “treatment” as used herein relates in its broadest sense to the administration of an LSD1 inhibitor (to a subject suffering from leukemia). In a more simplified form, the terms “response to treatment with an LSD1 inhibitor” and the like can be phrased “response to an LSD1 inhibitor” and the like.

Thus, the present invention relates in a related aspect to a method for the identification of a responding subject to an LSD1 inhibitor, said method comprising determining the level of one or more of the markers S100A12, VCAN, ITGAM, LY96, ANXA2, CD86, GPR65, CRISP9, LYZ and VIM in a sample from a subject suffering from leukemia, wherein an increased level of one or more of the markers S100A12, VCAN, ITGAM, LY96, ANXA2, CD86, GPR65, CRISP9, LYZ and VIM compared to a control is indicative for a responding subject. In other words, the present invention relates in a related aspect to a method for the identification of a responding subject to an LSD1 inhibitor, said method comprising determining the level of one or more of the markers S100A12, VCAN, ITGAM, LY96, ANXA2, CD86, GPR65, CRISP9, LYZ and VIM in a sample from a subject suffering from leukemia, wherein if the level of one or more of the markers S100A12, VCAN, ITGAM, LY96, ANXA2, CD86, GPR65, CRISP9, LYZ and VIM is increased compared to a control, the subject is responsive to the LSD1 inhibitor.

In a further related aspect, the present invention relates to a method of determining whether a proliferative diseased cell is responsive to an LSD1 inhibitor, said method comprising determining the level of one or more of the markers S100A12, VCAN, ITGAM, LY96, ANXA2, CD86, GPR65, CRISP9, LYZ and VIM in a sample from a subject suffering from leukemia, wherein an increased level of one or more of the markers S100A12, VCAN, ITGAM, LY96, ANXA2, CD86, GPR65, CRISP9, LYZ and VIM compared to a control is indicative for a responsive proliferative diseased cell. In other words, the present invention relates to a method of determining whether a proliferative diseased cell is responsive to an LSD1 inhibitor, said method comprising determining the level of one or more of the markers S100A12, VCAN, ITGAM, LY96, ANXA2, CD86, GPR65, CRISP9, LYZ and VIM in a sample from a subject suffering from leukemia, wherein if the level of one or more of the markers S100A12, VCAN, ITGAM, LY96, ANXA2, CD86, GPR65, CRISP9, LYZ and VIM is increased compared to a control, the proliferative diseased cell is responsive to the LSD1 inhibitor.

It is understood that the present invention aims at providing a companion diagnostic test using samples from subjects suffering from leukemia wherein the subjects receive a treatment with an LSD1 inhibitor.

Leukemia is a cancer of the body's blood-forming tissues. These tissues include the bone marrow and the lymphatic system. Leukemia often begins in the bone marrow. A normal bone marrow cell undergoes a change and becomes a type of leukemia cell. Once the marrow cell undergoes such a change, the leukemia cells can grow and survive better than normal cells. Thus, the leukemia cells crowd out or suppress the development of normal cells over time.

Different types of leukemia depend on the type of blood cell that becomes a cancer cell. For example, lymphoblastic leukemia is a cancer of the lymphoblasts.

White blood cells are the most common type of blood cell to become leukemic cancer cells. Thereby, leukemia results in high numbers of abnormal white blood cells. These abnormal white blood cells are not fully developed/differentiated and are called blasts. Red blood cells (erythrocytes) and platelets may also become leukemic cancer cells.

Diagnosis is typically made by blood tests or bone marrow biopsy. Symptoms of leukemia can include bleeding and bruising problems, feeling tired, fever, and an increased risk of infections. These symptoms are caused by a lack of normal blood cells.

Leukemia occurs most often in adults older than 55 years, but it is also the most common cancer in children younger than 15 years. Leukemia can be either acute or chronic. Acute leukemia is a fast-growing cancer that usually gets worse quickly. Chronic leukemia is a slower-growing cancer that gets worse slowly over time. The treatment and prognosis for leukemia depend on the type of blood cell affected and whether the leukemia is acute or chronic, among other factors.

For the purpose of the present invention, “leukemia” is preferably “myeloid leukemia”. “Myeloid leukemia” as used herein means any leukemia that has arisen from any cell of the developmental tree of myeloid cells (including multipotential hematopoietic stem cells, common myeloid progenitors, megakaryoblasts, erythroblasts, myeloblasts, mast cell progenitors, monocytes/macrophages, eosinophils, neutrophils, basophils, megakaryocytes/thrombocytes, erythrocytes, and mast cells, as well as cells that have arosen from other hematopoeietic lineages and that have undergone oncogenic transformation providing myeloid characteristics), both acute and chronic, including also mixed lineage/multilineage leukemias. Myeloid leukemia as used herein thus comprises, without being limited thereto, leukemias as classified in classes C92 to C94 of the International Classification of Diseases ICD-10 (online version 2016).

Most preferred herein is acute myeloid leukemia (AML). Acute myeloid leukemia (AML) is a cancer of the myeloid lineage of blood cells, characterized by the rapid growth of abnormal white blood cells that accumulate in the bone marrow and interfere with the production of normal blood cells. AML can occur in adults and children. It is the most common type of acute leukemia in adults. AML as used herein includes, inter alia, acute myelogenous leukemia, acute myeloblastic leukemia, acute granulocytic leukemia, and acute nonlymphocytic leukemia.

AML as used herein includes any leukemia classified as such according to any of the medically recognized past, current or future classification systems.

For example, “AML” as used herein includes leukemias of French-American-British (FAB) subtypes M0 to M7. The French-American-British (FAB) AML classification of 1976 (Proposals for the classification of the acute leukaemias. French-American-British (FAB) co-operative group. Bennett J M, Catovsky D, Daniel M T, Flandrin G, Galton D A, Gralnick H R, Sultan C. Br J Haematol. 1976 August; 33(4):451-8) and its subsequent revision (Proposed revised criteria for the classification of acute myeloid leukemia. A report of the French-American-British Cooperative Group. Bennett J M, Catovsky D, Daniel M T, Flandrin G, Galton D A, Gralnick H R, Sultan C. Ann Intern Med. 1985 October; 103(4):620-5) divided AMLs into 8 subtypes, based on morphologic and cytochemical features of the bone marrow leukemic blasts, including the type of cell from which the leukemia developed and how mature the cells were, among others.

| FAB subtype | Name |
|---|---|
| M0 | Undifferentiated AML |
| M1 | AML without maturation (poorly differentiated) |
| M2 | AML with maturation (more differentiated) |
| M3 | Acute promyelocytic leukemia |
| M4 | Acute myelomonocytic leukemia |
| | Subtype: |
| | M4 eos: Acute myelomonocytic leukemia with eosinophilia |
| M5 | Acute monocytic leukemia |
| | Subtypes: |
| | M5a: Acute monoblastic leukemia - poorly differentiated |
| | M5b: Acute monocytic leukemia - more differentiated |
| M6 | Acute erythroblastic leukemia |
| | Subtypes: |
| | M6a: Erythroleukemia |
| | M6b: Pure erythroid leukemia |
| M7 | Acute megakaryoblastic leukemia |

In particular, M4, M5, and M6 FAB subtypes correspond to C92.5, C93.0, and C94.0 WHO ICD-10 classes (online version 2016):

C92.5 Acute myelomonocytic leukaemia

AML M4
AML M4 Eo with inv(16) or t(16; 16)

C93.0 Acute monoblastic/monocytic leukaemia

AML M5a
AML M5b
AML M5

C94.0Acute Erythroid Leukaemia

Acute myeloid leukaemia M6 (a)(b)

Erythroleukaemia

The morphologic subtypes of AML also include rare types not included in the FAB system, such as acute basophilic leukemia, which was proposed as a ninth subtype, M8.

For example, “AML” as used herein includes the following categories: AML with recurrent genetic abnormalities, AML with myelodysplasia related changes, therapy related myeloid neoplasms, AML not otherwise specified (NOS), myeloid sarcoma, and myeloid proliferations related to Down Syndrome; or any subcategory thereof defined in the WHO Classification of myeloid neoplasms and acute leukemia (Arber D A, Orazi A, Hasserjian R, Thiele J, Borowitz M J, Le Beau M M, Bloomfield C D, Cazzola M, Vardiman J W. Blood 2016 May 19; 127(20):2391-405).

Particularly preferred herein is AML subtype M4 or M5, as assessed/determined according to French-American-British (FAB) classification. French-American-British (FAB) subtype M4 corresponds to C92.5 and FAB subtype M5 corresponds to C93.0 of WHO classification ICD-10 (version 2016), respectively.

Preferably, the AML herein is acute myelomonocytic leukemia, acute monoblastic leukemia or acute monocytic leukemia.

The term “subject suffering from leukemia” as used herein refers to an individual suffering from leukemia. The terms “subject” and “individual” and “patient” are used interchangeably herein. Preferably, the subject is a human. A “subject suffering from leukemia” typically shows/has (clinical) symptoms as described above, e.g. bleeding, bruising problems, feeling tired, fever, and/or an increased risk of infections. These symptoms are normally caused by a lack of normal blood cells. In addition/in the alternative, the “subject suffering from leukemia” has been (clinically) diagnosed for leukemia e.g. by a blood test or by a bone marrow test. By looking at a sample of the blood, it can be determined if a subject suspected of suffering from leukemia has abnormal levels of white blood cells or platelets which indicates that the subject suffers from leukemia. The bone marrow sample can e.g. be taken from the hipbone. By looking at a sample of the bone marrow the presence and/or percentage of leukemia cells can be determined which in turn indicates that the subject suffers from leukemia. A subject that has thus been/is thus diagnosed to suffer from leukemia can be termed a “leukemia patient”. Preferably, the leukemia patient is a human leukemia patient.

The methods of the invention comprise determining the level of one or more of the markers S100A12, VCAN, ITGAM, LY96, ANXA2, CD86, GPR65, CRISP9, LYZ and VIM. These markers per se are well known in the art and also described herein below.

The following aliases for these markers are known:

S100a12 has the following aliases according to GeneCards:

S100 Calcium Binding Protein A12, Extracellular Newly identified RAGE-Binding Protein, S100 Calcium-Binding Protein A12 (Calgranulin C), Migration Inhibitory Factor-Related Protein 6, Calcium-Binding Protein In Amniotic Fluid 1, Neutrophil S100 Protein, Calgranulin-C, EN-RAGE, CAAF1, MRP-6, CGRP, CAGC, P6, S100 Calcium Binding Protein A12 (Calgranulin C), S100 Calcium-Binding Protein A12, Calgranulin C, Calcitermin, ENRAGE, MRP6

Vcan has the following aliases according to GeneCards:

Versican, Chondroitin Sulfate Proteoglycan 2, Chondroitin Sulfate Proteoglycan Core Protein 2, Glial Hyaluronate-Binding Protein, Large Fibroblast Proteoglycan, Versican Proteoglycan, CSPG2, GHAP, PG-M, ERVR, WGN1, WGN Itgam has the following aliases according to GeneCards:

CD11b, Integrin Subunit Alpha M, Integrin, Alpha M (Complement Component 3 Receptor 3 Subunit), Cell Surface Glycoprotein MAC-1 Subunit Alpha, Complement Component 3 Receptor 3 Subunit, CD11 Antigen-Like Family Member B, Leukocyte Adhesion Receptor MO1, CR-3 Alpha Chain, CR3A, Integrin, Alpha M (Complement Component Receptor 3, Alpha; Also Known As CD11b (P170), Macrophage Antigen Alpha Polypeptide), Neutrophil Adherence Receptor Alpha-M Subunit, Macrophage Antigen Alpha Polypeptide, Neutrophil Adherence Receptor, Antigen CD11b (P170), CD11b Antigen, MAC-1, MAC1A, SLEB6, MO1A

Ly96 has the following aliases according to GeneCards:

Lymphocyte Antigen 96, Protein MD-2, ESOP-1, Ly-96, MD2, Myeloid Differentiation Protein-2, ESOP1, MD-2

Anxa2 has the following aliases according to GeneCards:

Annexin A2, Annexin II Placental Anticoagulant Protein IV, Calpactin I Heavy Chain, Calpactin-1 Heavy Chain, Chromobindin-8, Lipocortin II, Protein I, Annexin-2, ANX2L4, PAP-IV, CAL1H, LPC2D, ANX2, P36 Epididymis Secretory Protein Li 270, Calpactin I Heavy Polypeptide, Chromobindin 8, HEL-S-270, L IP2, LPC2

Cd86 has the following aliases according to GeneCards:

CD86 Molecule, CD86 Antigen (CD28 Antigen Ligand 2, B7-2 Antigen), CTLA-4 Counter-Receptor B7.2, CD28LG2, FUN-1, BU63, B70, B-Lymphocyte Activation Antigen B7-2, B-Lymphocyte Antigen B7-2, Activation B7-2 Antigen, CD86 Antigen, LAB72, B7-2, B7.2

Gpr65 has the following aliases according to GeneCards:

G Protein-Coupled Receptor 65, T-Cell Death-Associated Gene 8 Protein, G-Protein Coupled Receptor 65, TDAG8, HTDAG8

Crisp9 has the following aliases according to GeneCards:

PI16, Peptidase Inhibitor 16, Cysteine-Rich Secretory Protein 9, Protease Inhibitor 16, PSP94-Binding Protein, PSPBP, Microseminoprotein, Beta-Binding Protein, Beta-Binding Protein, Microseminoprotein, MSMBBP, CD364

LYZ has the following aliases according to GeneCards:

Lysozyme, 1, 4-Beta-N-Acetylmuramidase C, EC 3.2.1.17, LZM, Lysozyme (Renal Amyloidosis), Renal Amyloidosis, C-Type Lysozyme, Lysozyme F1, LYZF1

Vim has the following aliases according to GeneCards:

Vimentin, Epididymis Luminal Protein 113, CTRCT30, HEL113

Further, also Camsap2 and Ctsg are known.

Camsap2has the following aliases according to GeneCards:

Calmodulin Regulated Spectrin Associated Protein Family Member 2, Calmodulin Regulated Spectrin-Associated Protein Family, Member 2, Calmodulin-Regulated Spectrin-Associated Protein 1-Like Protein 1, CAMSAP1L1, Calmodulin Regulated Spectrin-Associated Protein 1-Like 1, KIAA1078

Ctsg has the following aliases according to GeneCards:

Cathepsin G, CG, EC 3.4.21.20, EC 3.4.21,CATG

Aliases for each of the 2 endogenous control markers as employed herein are as follows:

Gapdh has the following aliases according to GeneCards:

Glyceraldehyde-3-Phosphate Dehydrogenase, Peptidyl-Cysteine S-Nitrosylase GAPDH, EC 1.2.1.12,

GAPD, Epididymis Secretory Sperm Binding Protein Li 162eP, Aging-Associated Gene 9 Protein, HEL-S-162eP, EC 2.6.99.-, EC 1.2.1, G3PD

Hprt has the following aliases according to GeneCards:

Hypoxanthine Phosphoribosyltransferase 1, EC 2.4.2.8, HGPRTase, HGPRT, HPRT1, Hypoxanthine-Guanine Phosphoribosyltransferase 1, Testicular Tissue Protein Li 89, Lesch-Nyhan Syndrome

As used herein, the names of the markers can interchangeably be written in capital letters or small case letters. Therefore, VCAN is equivalent to Vcan, S100A12 is equivalent to S100a12, LY96 is equivalent to Ly96 etc

Public data base entries:

DNA and protein sequences of human S100A12, VCAN, ITGAM, LY96, ANXA2, CD86, GPR65, CRISP9, LYZ and VIM, as well as of Camsap2, Ctsg, Gapdh, and Hprt1 have been previously reported, see GenBank Numbers (NCBI-GenBank Flat File Release 216.0, Oct. 15, 2016) and UniProtKB/Swiss-Prot Numbers (Knowledgebase Release 2016_09) listed below, each of which is incorporated herein by reference in its entirety for all purposes. Such sequences can be used to design procedures for determining and analysis of the level of S100A12, VCAN, ITGAM, LY96, ANXA2, CD86, GPR65, CRISP9, LYZ and VIM, as well as of Camsap2, Ctsg, Gapdh, and Hprt1 by ways known to one skilled in the art.

| Name | NCBI Reference Sequence | UniProtKB/Swiss-Prot |
|---|---|---|
| S100a12 | NM_005621.1 | S10AC_HUMAN, P80511 |
| Vcan | NM_001126336.2 | CSPG2_HUMAN, P13611 |
| | NM_001164097.1 | (incl. 5 isoforms: |
| | NM_001164098.1 | P13611-1 to P13611-5) |
| | NM_004385.4 | |
| Itgam | NM_001145808.1 | ITAM_HUMAN, P11215 |
| | NM_000632.3 | (incl. 2 isoforms: |
| | | P11215-1 and P11215-2) |
| Ly96 | NM_015364.4 | LY96_HUMAN, Q9Y6Y9 |
| | NM_001195797.1 | (incl. 2 isoforms: |
| | | Q9Y6Y9-1 and Q9Y6Y9-2) |
| Anxa2 | NM_001002858.2 | ANXA2_HUMAN, P07355 |
| | NM_001002857.1 | (incl. 2 isoforms: |
| | NM_001136015.2 | P07355-1 and P07355-2) |
| | NM_004039.2 | |
| Cd86 | NM_001206924.1 | CD86_HUMAN, P42081 |
| | NM_001206925.1 | (incl. 6 isoforms: |
| | NM_006889.4 | P42081-1 to P42081-6) |
| | NM_175862.4 | |
| | NM_176892.1 | |
| Gpr65 | NM_003608.3 | PSYR_HUMAN, Q8IYL9 |
| Crisp9 | NM_153370.2 | PI16_HUMAN, Q6UXB8 |
| | NM_001199159.1 | (incl. 2 isoforms: |
| | | Q6UXB8-1 and Q6UXB8-2) |
| Lyz | NM_000239.2 | LYSC_HUMAN, P61626 |
| Vim | NM_003380.3 | VIME_HUMAN, P08670 |
| Camsap2 | NM_203459.2 | CAMP2_HUMAN, Q08AD1 |
| Ctsg | NM_001911.2 | CATG_HUMAN, P08311 |
| Gapdh | NM_002046.5 | G3P_HUMAN, P04406 |
| Hprt1 | NM_000194.2 | HPRT_HUMAN, P00492 |

Exemplary amino acid sequences and nucleotide sequences of human S100A12, VCAN, ITGAM, LY96, ANXA2, CD86, GPR65, CRISP9, LYZ, VIM, CAMSAP2, CTSG, Gapdh, and Hprt1 are shown in SEQ ID NO: 1 to 28 herein. The following table allocates the markers and the respective sequences:

| | Nucleotide sequence | Amino acid sequence |
|---|---|---|
| | (SEQ ID NO) | (SEQ ID NO) |
| S100A12 | 1 | 2 |
| VCAN | 3 | 4 |
| ITGAM | 5 | 6 |
| LY96 | 7 | 8 |
| ANXA2 | 9 | 10 |
| CD86 | 11 | 12 |
| GPR65 | 13 | 14 |
| CRISP9 | 15 | 16 |
| LYZ | 17 | 18 |
| VIM | 19 | 20 |
| CAMSAP2 | 21 | 22 |
| CTSG | 23 | 24 |
| Gapdh | 25 | 26 |
| Hprt1 | 27 | 28 |

The methods of the invention can comprise determining the level of 2 or more, 3 or more, 4 or more, 5 or more, 6 or more, 7 or more, 8 or more, or 9, or 10 of the markers S100A12, VCAN, ITGAM, LY96, ANXA2, CD86, GPR65, CRISP9, LYZ and VIM. In a more preferred aspect, the methods of the invention comprise determining the level of all of the markers S100A12, VCAN, ITGAM, LY96, ANXA2, CD86, GPR65, CRISP9, LYZ and VIM (i.e. of a combination of S100A12, VCAN, ITGAM, LY96, ANXA2, CD86, GPR65, CRISP9, LYZ and VIM).

Preferably, the methods of the invention comprise determining the level of all of the markers S100A12, VCAN, ITGAM, LY96, ANXA2, CD86, GPR65, CRISP9, LYZ and VIM (i.e. of a combination of S100A12, VCAN, ITGAM, LY96, ANXA2, CD86, GPR65, CRISP9, LYZ and VIM), wherein a subject/diseased cell is identified as responsive to (treatment with) an LSD1 inhibitor if at least 6 (e.g. 6, 7, 8, 9 or all) of said markers are increased compared to a control, and preferably if at least 7 (e.g. 7, 8, 9 or all) of said markers are increased compared to a control.

The methods of the invention can comprise determining the level of one or more, of the markers S100A12, VCAN, ITGAM, LY96, ANXA2, CD86, GPR65, CRISP9, and LYZ. The methods of the invention can comprise determining the level of one or more, 2 or more, 3 or more, 4 or more, 5 or more, 6 or more, 7 or more, 8 or 9, of the markers S100A12, VCAN, ITGAM, LY96, ANXA2, CD86, GPR65, CRISP9, and LYZ. In a more preferred aspect the methods of the invention comprise determining the level of all of the markers S100A12, VCAN, ITGAM, LY96, ANXA2, CD86, GPR65, CRISP9, and LYZ (i.e. of a combination of S100A12, VCAN, ITGAM, LY96, ANXA2, CD86, GPR65, CRISP9, and LYZ).

In one aspect, the present invention relates to a method for monitoring the response to treatment with an LSD1 inhibitor in a subject suffering from leukemia, said method comprising determining the level of one or more of the markers S100A12, VCAN, ITGAM, LY96, ANXA2, CD86, GPR65, CRISP9, and LYZ, in a sample from said subject, wherein an increased level of one or more of the markers S100A12, VCAN, ITGAM, LY96, ANXA2, CD86, GPR65, CRISP9, and LYZ compared to a control is indicative for response to treatment.

In one aspect, the present invention relates to a method for the identification of a responding subject to treatment with an LSD1 inhibitor, said method comprising determining the level of one or more of the markers S100A12, VCAN, ITGAM, LY96, ANXA2, CD86, GPR65, CRISP9, and LYZ in a sample from a subject suffering from leukemia, wherein an increased level of one or more of the markers S100A12, VCAN, ITGAM, LY96, ANXA2, CD86, GPR65, CRISP9, and LYZ compared to a control is indicative for a responding subject.

In one aspect, the present invention relates to a method of determining whether a proliferative diseased cell is responsive to treatment with an LSD1 inhibitor, said method comprising determining the level of one or more of the markers S100A12, VCAN, ITGAM, LY96, ANXA2, CD86, GPR65, CRISP9, and LYZ in a sample from a subject suffering from leukemia, wherein an increased level of one or more of the markers S100A12, VCAN, ITGAM, LY96, ANXA2, CD86, GPR65, CRISP9, and LYZ compared to a control is indicative for a responsive proliferative diseased cell.

Preferably, the methods of the invention comprise determining the level of all of the markers S100A12, VCAN, ITGAM, LY96, ANXA2, CD86, GPR65, CRISP9, and LYZ (i.e. of a combination of S100A12, VCAN, ITGAM, LY96, ANXA2, CD86, GPR65, CRISP9, and LYZ), wherein a subject/diseased cell is identified as responsive to (treatment with) an LSD1 inhibitor if at least 6 (e.g. 6, 7, 8, 9 or all) of said markers are increased compared to a control, and preferably if at least 7 (e.g. 7, 8, 9 or all) of said markers are increased compared to a control.

In a preferred aspect, the methods of the invention comprise determining the level of one or more, 2 or more, 3 or more, 4 or more, 5 or 6 of the markers S100A12, VCAN, ITGAM, LY96, ANXA2, and CD86. In a particularly preferred aspect, the methods of the invention comprise determining the level of all of the markers S100A12, VCAN, ITGAM, LY96, ANXA2, and CD86 (i.e. a combination of markers S100A12, VCAN, ITGAM, LY96, ANXA2, and CD86 is used).

In one preferred aspect, the present invention relates to a method for monitoring the response to treatment with an LSD1 inhibitor in a subject suffering from leukemia, said method comprising determining the level of one or more of the markers S100A12, VCAN, ITGAM, LY96, ANXA2, and CD86, in a sample from said subject, wherein an increased level of one or more of the markers S100A12, VCAN, ITGAM, LY96, ANXA2, and CD86 compared to a control is indicative for response to treatment.

In one preferred aspect, the present invention relates to a method for the identification of a responding subject to treatment with an LSD1 inhibitor, said method comprising determining the level of one or more of the markers S100A12, VCAN, ITGAM, LY96, ANXA2, and CD86 in a sample from a subject suffering from leukemia, wherein an increased level of one or more of the markers S100A12, VCAN, ITGAM, LY96, ANXA2, and CD86 compared to a control is indicative for a responding subject.

In one preferred aspect, the present invention relates to a method of determining whether a proliferative diseased cell is responsive to treatment with an LSD1 inhibitor, said method comprising determining the level of one or more of the markers S100A12, VCAN, ITGAM, LY96, ANXA2, and CD86 in a sample from a subject suffering from leukemia, wherein an increased level of one or more of the markers S100A12, VCAN, ITGAM, LY96, ANXA2, and CD86 compared to a control is indicative for a responsive proliferative diseased cell.

As explained above and shown in the appended examples, the level of markers Ly96 and ITGAM in blood has been confirmed herein to correlate with the effect of treatment with an LSD1 inhibitor on blast number in bone marrow, particularly in samples from patients of the AML M4/M5 subtype. Thus, if it is desired to monitor the levels of blast cells in the bone marrow, determining the level of markers Ly96 and/or ITGAM (preferably the level in a blood sample from said subject, particularly a peripheral blood sample from said subject) is particularly envisaged.

In one aspect, the present invention relates to a method for monitoring the response to treatment with an LSD1 inhibitor in a subject suffering from leukemia, said method comprising determining the level of the markers Ly96 and/or ITGAM, in a sample from said subject, wherein an increased level of the markers Ly96 and/or ITGAM compared to a control is indicative for response to treatment.

In one aspect, the present invention relates to a method for the identification of a responding subject to treatment with an LSD1 inhibitor, said method comprising determining the level of the markers Ly96 and/or ITGAM in a sample from a subject suffering from leukemia, wherein an increased level of the markers Ly96 and/or ITGAM compared to a control is indicative for a responding subject.

In one aspect, the present invention relates to a method of determining whether a proliferative diseased cell is responsive to treatment with an LSD1 inhibitor, said method comprising determining the level of the markers Ly96 and/or ITGAM in a sample from a subject suffering from leukemia, wherein an increased level of the markers Ly96 and/or ITGAM compared to a control is indicative for a responsive proliferative diseased cell.

It is preferred in this context that the level of Ly96 and ITGAM is determined.

As demonstrated herein, the herein provided markers are not only useful to monitor response to an LSD1 inhibitor or identify responders to treatment with an LSD1 inhibitor, but are also useful for predicting/assessing whether a subject is at risk of developing/suffering from a differentiation syndrome (DS). The subject is suffering from leukemia and is treated with an LSD1 inhibitor. In this context the term “monitoring response” or “identifying a responding subject” can include or be predicting/assessing whether a subject is at risk of developing a differentiation syndrome (DS). As demonstrated herein, biomarkers S100A12 and VCAN showed an exacerbated (18 to 550-fold) up-regulation in patients that developed differentiation syndrome. Importantly, this up-regulation could be observed up to 2 weeks prior to the clinical diagnosis of the differentiation syndrome. Thus, S100A12 and VCAN are a useful tool to early monitor the risk of developing a differentiation syndrome in leukemia patients receiving treatment with an LSD1 inhibitor (e.g. ORY-1001), particularly in AML M4/M5 subtypes.

In accordance with the above, the present invention relates in one aspect to a method for predicting/assessing whether a subject is at risk of developing/suffering from a differentiation syndrome (DS), said method comprising determining the level of one or more of the markers S100A12, VCAN, ITGAM, LY96, ANXA2, CD86, GPR65, CRISP9, LYZ and VIM, in a sample from said subject, wherein an increased level of one or more of the markers S100A12, VCAN, ITGAM, LY96, ANXA2, CD86, GPR65, CRISP9, LYZ and VIM compared to a control is indicative for an increased risk of developing/suffering from a differentiation syndrome (DS). The subject is suffering from leukemia and is treated with an LSD1 inhibitor. In a preferred aspect, the present invention relates to a method for predicting/assessing whether a subject is at risk of developing/suffering from a differentiation syndrome (DS), said method comprising determining the level of one or more of the markers S100A12 and VCAN in a sample from said subject, wherein an increased level of one or more of the markers S100A12 and VCAN compared to a control is indicative for a(n) (increased) risk of developing/suffering from a differentiation syndrome (DS). The subject is suffering from leukemia and is treated with an LSD1 inhibitor.

In one aspect, the present invention relates to a method for the identification of a subject that is at risk of developing/suffering from a differentiation syndrome (DS), said method comprising determining the level of one or more of the markers S100A12, VCAN, 1TGAM, LY96, ANXA2, CD86, GPR65, CRISP9, LYZ and VIM in a sample from a subject suffering from leukemia, wherein an increased level of one or more of the markers S100A12, VCAN, ITGAM, LY96, ANXA2, CD86, GPR65, CRISP9, LYZ and VIM compared to a control is indicative for a(n) (increased) risk of developing/suffering from a differentiation syndrome (DS). The subject is suffering from leukemia and is treated with an LSD1 inhibitor (i.e. is undergoing treatment with an LSD1 inhibitor). In a preferred aspect, the present invention relates to a method for the identification of a subject that is at risk of developing/suffering from a differentiation syndrome (DS), said method comprising determining the level of one or more of the markers S100A12 and VCAN in a sample from a subject suffering from leukemia, wherein an increased level of one or more of the markers S100A12 and VCAN compared to a control is indicative for a(n) (increased) risk of developing/suffering from a differentiation syndrome (DS).

In a further aspect, the present invention relates to a method for monitoring the risk of developing/suffering from a differentiation syndrome in a subject with/suffering leukemia receiving treatment/being treated with an LSD1 inhibitor, which comprises determining the level of one or more of the markers S100A12, VCAN, ITGAM, LY96, ANXA2, CD86, GPR65, CRISP9, LYZ and VIM, in a sample from said subject, wherein an increased level of one or more of the markers S100A12, VCAN, ITGAM, LY96, ANXA2, CD86, GPR65, CRISP9, LYZ and VIM compared to a control is indicative for an increased risk of developing/suffering from a differentiation syndrome (DS). In a preferred aspect the present invention relates to a method for monitoring the risk of developing/suffering from a differentiation syndrome in a subject with/suffering leukemia receiving treatment/being treated with an LSD1 inhibitor, which comprises determining the level of one or more of the markers S100A12 and VCAN, in a sample from said subject, wherein an increased level of one or more of the markers S100A12 and VCAN compared to a control is indicative for an increased risk of developing/suffering from a differentiation syndrome (DS).

In the context of the methods for assessing the risk for developing/suffering from a differentiation syndrome, a risk of developing DS is identified if the level of one or more of the markers to be used herein, particularly of S100A12 and/or VCAN, is increased at least 8-fold in comparison to a control, and the risk is even higher if the level of said markers is increased by at least 16-fold in comparison to a control.

In this context, the treatment of said subject with said LSD1 inhibitor can be adapted if the level of one or more of the markers to be used herein, particularly of S100A12 and/or VCAN, is increased in comparison to a control. For example, the adaption of the treatment may comprise administering a decreased amount of the LSD1 inhibitor for a certain period of the treatment, a treatment stop of the LSD1 inhibitor, or the administration of an additional therapy (e.g. a therapy treating, preventing or ameliorating (the side-effects of) the differentiation syndrome).

The type of sample to be used herein is not limited as long as leukemic cells/leukemic cancer cells are present in the sample. For example, tissues invaded by leukemic tumor cells may be used. Also a bone marrow sample from a subject can be used. Yet, the use of blood samples is generally preferred herein, and peripheral blood samples are particularly preferred.

It is understood that the cancer cell(s)/proliferative diseases cell(s) to be evaluated/assessed/scrutinized may be part of a sample (like a blood sample or a bone marrow sample). In relation to leukemia, the term “cancer cell(s)” can refer to (a) “proliferative diseased cell(s)”. In this context, also the level of (a) marker(s) of the invention in cells other than “proliferative diseased cell(s)” from a given sample (like a bone marrow sample or a blood sample) may be determined without deferring from the gist of this invention. In this context, it can be contemplated that a prior isolation (by sorting, MACS, etc.) of myeloid cells (e.g. from blood) is performed to enrich for myeloid cells and, hence, also for “proliferative diseased cell(s)” in the sample. “prior isolation” means “isolation” prior to determining the level of one or more of the markers of the invention.

The sample (e.g. the sample comprising the at least one “proliferative diseased cell”) can be obtained from a subject. In one aspect, the methods of the invention can comprise a step of obtaining a sample from a subject. The obtaining step is prior to the “determining the level of one or more of the markers of the invention” and prior to a potential step of isolation (by sorting, MACS, etc.) of myeloid cells from said obtained sample, if applicable.

The term “proliferative diseased cell(s)” as used herein refers to a leukemic cell/leukemic cancer cell, for example (an) immature white blood cell(s)/immature leukocye(s)/blast(s).

The term “responsiveness” (and likewise “respond” and grammatical variants thereof) as used herein means that (a) proliferative diseased cell/cancer cell and/or a patient as defined herein responds to or has an increased likelihood of responding to an LSD1 inhbitor. The term “response” as used in the context of the present invention (e.g. in the context of response to (treatment with) an LSD1 inhibitor or in the context of response of a subject or diseased cell to (treatment with) an LSD1 inhibitor) means: (i) blast differentiation in bone marrow and/or peripheral blood, and/or (ii) a decrease in blast counts in bone marrow and/or peripheral blood; preferentially, “response” includes a decrease in blast counts in bone marrow and/or peripheral blood, most preferably “response” means: (i) blast differentiation in bone marrow and/or peripheral blood, and (ii) a decrease in blast counts in bone marrow and/or peripheral blood. Ideally, a “response” translates into a complete remission (CR), morphologic complete remission with incomplete blood count recovery (CRi), morphologic leukemia-free state, cytogenetic complete remission (CRc), molecular complete remission (CRm), or partial remission (PR) of said subject, which can be assessed as known in the art (see e.g. H. Döhner et al, Blood. 2010 Jan. 21; 115(3):453-74. doi: 10.1182/blood-2009-07-235358. Epub 2009 Oct. 30; B D Cheson et al, J Clin Oncol. 2003 Dec. 15; 21(24):4642-9).

The herein provided methods can be useful in a therapeutic setting, i.e. if a patient suffers from leukemia and is treated with an LSD1 inhibitor. In other words, if leukemia has already been diagnosed and the subject is undergoing anti-leukemia therapy is, the methods of the present invention can allow stratification of subjects which can benefit from therapy with an LSD1 inhbitor. If, for example, one or more of the markers of the invention is increased in a sample, the patient can be eligible for (ongoing) therapy with an LSD1 inhibitor. For such patients the LSD1 inhibitor might be the sole anti-cancer therapy or LSD1 inhibitor might be administered as co-therapy (e.g. in combination with a second (or yet further) LSD1 inhibitor or in combination with conventional therapy). The methods of the present invention may also be useful in order to stratify patients which cannot benefit from therapy with an LSD1 inhibitor.

A person skilled in the art will appreciate that a positive test that the level of one or more of the markers of the invention is increased does not necessarily translate 1:1 into a successful treatment of leukemia. However, by these methods sub-groups of patients/subjects are identified that have a higher chance of a positive clinical response (=show a better response rate) to a treatment with an LSD1 inhibitor, as compared to the sub-group of patients not showing these positive test results. In other words, a positive result indicates that the subject/patient has a higher chance to respond to treatment with an LSD1 inhibitor as compared to a subject/patient with no increased level of one or more of the markers of the invention.

In accordance with the present invention, the sample is obtained (or is to be obtained) from the subject after the initiation of the treatment with the LSD1 inhibitor. In other words, the sample is obtained (or is to be obtained) from the subject during the treatment with the LSD1 inhibitor and, optionally, after the treatment with the LSD1 inhibitor (after the treatment is terminated). For example, the sample is obtained (or is to be obtained) from the subject at day 3 or at a subsequent day after the initiation of the treatment with the LSD1 inhibitor (i.e. at any one day during the treatment with an LSD1 inhibitor, preferably starting at day 3 of the treatment). The sample can also be obtained earlier, e.g. at day 1 or day 2. As non-limiting examples, the sample is (to be) obtained at day 3, 4, 5, 6, 7, 8, 9, 10, 11, 12, 13, 14, 15, 16, 17, 18, 19, 20, 21, 22, 23, 24, 25, or 26 etc. days after the initiation of the treatment with said LSD1 inhibitor. The sample can also be obtained earlier, e.g. at day 1 or day 2 after the initiation of the treatment with said LSD1 inhibitor. The “initation of the treatment” would be at “day 1”.

It is contemplated herein that several samples from said same subject can be obtained, e.g. samples at different days after the initiation of the treatment (e.g. the first sample is obtained not earlier than at day 3, and (an) additional sample(s) is optionally obtained at (a) later day(s) during the treatment). Generally, the methods of the invention can comprise in accordance with the above determining the level of one or more of the markers of the invention in a second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth etc. sample.

It is also envisaged herein that several samples can be obtained from the subject on the same day at different time points (hours). For example, two, 3, 4, 5, or more sample(s) can be obtained from the subject on the same day. As a further non-limiting example, the multiple sample are (to be) obtained at day 3, 4, 5, 6, 7, 8, 9, 10, 11, 12, 13, 14, 15, 16, 17, 18, 19, 20, 21, 22, 23, 24, 25, and/or 26 etc. days after the initiation of the treatment with said LSD1 inhibitor.

As mentioned, an increased level of one or more of the markers S100A12, VCAN, ITGAM, LY96, ANXA2, CD86, GPR65, CRISP9, LYZ and VIM, indicates a response. Whether there is an increase is determined in comparison to a control, preferably a control for said marker.

As used in context of the methods of the present invention, a non-limiting example of a “control” (for a specific marker) can be a “non-responder” control, for example the level of a specific marker to be used herein in a sample/cell/tissue obtained from one or more healthy subjects or obtained from one or more subjects suffering from leukemia but already known to be not responsive to an LSD1 inhibitor. Another example for a “non-responder” control is the level of specific marker to be used herein in a cell line/sample/cell/tissue that shows no response to an LSD1 inhibitor in an ex-vivo/in vitro test. Another non-limiting example of a “control” is an “internal standard”, for example purified or synthetically produced RNA, proteins and/or peptides or a mixture thereof, where the amount of each RNA/protein/peptide is gauged by using the “non-responder” control described above. The control may also be the level of a specific marker to be used herein in a sample/cell/tissue obtained from said same subject suffering from leukemia, provided that the sample/cell/tissue does not contain proliferative diseased cells as defined herein. The control may also be the level of a specific marker to be used herein in a sample/cell/tissue obtained from an subject suffering from leukemia that has been obtained prior to the development or diagnosis of said leukemia.

Preferably, a “control” for a specific marker to be used herein (i.e. S100A12, VCAN, ITGAM, LY96, ANXA2, CD86, GPR65, CRISP9, LYZ or VIM), is the level of said specific marker (i.e. S100A12, VCAN, ITGAM, LY96, ANXA2, CD86, GPR65, CRISP9, LYZ or VIM, respectively), determined in a sample of said same subject prior to the initiation of treatment with the LSD1 inhibitor. In other words, the control is the “base line” level of said marker in a sample from a subject suffering from leukemia before the subject has received treatment with an LSD1 inhibitor. For example, if the level of the marker S100A12 is determined in a sample from a subject suffering from leukemia after the inititation of treatment with the LSD1 inhibitor, the control for said marker S100A12 is the level of said marker S100A12 determined in a sample of said same subject prior to the initiation of treatment with said LSD1 inhibitor. This explanation and definition applies mutatis mutandis to marker(s) VCAN, ITGAM, LY96, ANXA2, CD86, GPR65, CRISP9, LYZ and VIM, respectively.

It is contemplated herein that the level of one or more of the markers S100A12, VCAN, ITGAM, LY96, ANXA2, CD86, GPR65, CRISP9, LYZ and VIM, is at least 1.3-fold, preferably at least 2-fold increased in comparison to a control.

Particular in relation to assessing the risk for developing a differentiation syndrome the level of one or more of the markers to be used herein, particularly of S100A12 and/or VCAN, is at least 8-fold (e.g. at least 16-fold) increased in comparison to a control.

The fold change herein is defined as the ratio of the level of the biomarker in the sample relative to the control. A fold change of 2, or 2-fold increase in the sample over the control means that the level of the biomarker in the sample was twice as high as the level in the control, a fold change of 0.5, or 2-fold decrease in the sample over the control means that the level of the biomarker in the sample was half as the level in the control. In a preferred embodiment of the method, the control is a sample obtained from the patient at baseline, i.e. prior to the administration of the first dose of LSD1 inhibitor.

The fold change can be calculated as the ratio of the biomarker's gene expression level in the sample relative to the biomarker's gene expression level in the control. Different methods have been described to assess relative levels of biomarker's gene expression. For example, the level of the biomarker in the sample relative to the control can be assessed by qRT-PCR. In the exponential phase of the amplification reaction, the intensity of the fluorescence is directly proportional to the quantity of PCR product formed. In qRT-PCR analysis, the fold change is calculated as 2̂(−ΔCp) or preferably as 2̂(−ΔΔCp)), where Cp is calculated applying the Second Derivative Maximum (SDM) cycle values; or as 2̂(−ΔC_{T}) or preferably as 2̂(−ΔΔC_{T}), where C_{T} is the threshold cycle value, or as 2̂(−ΔCq) 2̂(−ΔΔCq), where C_{T} is is the quantification cycle values.

For example, the LightCycler® 480 Software determines the “crossing point” (Cp), i.e. the point where the reaction's fluorescence reaches the maximum of the second derivative of the amplification curve, which corresponds to the point where the acceleration of the fluorescence signal is at its maximum. The Cp values reflect the target mRNA concentration in the original RNA sample. Differences in Cp values (ΔCp) for a gene X of interest in a given sample relative to a control sample reflect changes in mRNA concentration of the gene X in a given amount of total RNA in the respective sample, and are calculated as:

ΔCp, gene X=Cp(sample, gene X)−Cp(control, gene X)

To compensate for errors in the determination of RNA concentration or efficiency of 1^{st} strand synthesis or amplification, an endogenous reference gene is usually assessed in parallel to the gene X of interest for normalization, and the ΔΔCp is then calculated as:

ΔΔCp, gene X=[Cp(sample, gene X)−Cp(sample, reference gene)]−[Cp(control, gene X)−Cp(control, reference gene)]

The fold change in mRNA concentration is calculated as 2^{-ΔΔCp}, a negative ΔΔCp representing an increase in the expression level, and vice versa.

Microarray hybridization using chips or slides covered with probes to interrogate biomarkers can also be used to assess gene expression levels. In two-colour microarray analysis the fold change is calculated as the ratio between the signal intensities generated by the amplified and/or labeled nucleic acid derived from the RNA of the sample, labeled with one fluorophore; and the amplified and/or labeled nucleic acid derived from the RNA of the control, labeled with a second fluorophore, at the position of the biomarker probe. The ratio is frequently calculated after data processing of the raw signal intensities, including global normalization, compensation of spatial deviation and background subtraction. Microarray data are also frequently expressed as log2(ratio of the signal intensity of the marker in the sample/relative to the control). Microarray analysis can also be performed by using independent single colour hybridizations of the amplified and/or labeled RNAs derived from the sample and from the control, and by calculating the ratio between the ratio of the signal intensities in silico. Levels can also be calculated from the signals of multiple probes interrogating the biomarkers, and the raw signal intensities can be corrected by subtraction of the background or signal for a mismatch probe. Other techniques used to assess differential gene expression include RNA sequencing; in this case the expression level of a biomarker in a sample is determined by counting the amount of sequence reads corresponding to the biomarker relative to the total amount of sequence reads in the sample, and the fold change is calculated as the ratio of the relative level of the biomarker in the sample and the control. Other methods that can be used to measure RNA levels include digital PCR and nanopore sequencing.

The fold change can also be calculated from the ratio of the biomarker's protein level in the sample and of the biomarker's protein level in the control. Biomarker protein levels can be measured using immune based protein detection techniques including protein microarrays, colorimetric or chemoluminescent ELISA; or proximity assays including the Förster/Resonance Energy Transfer (FRET), AlphaLISA, DELFIA, and proximity ligation assays (protein PCR), or fluorescence activated cell sorting (FACS). Immune agents used to detect the protein can include biomarker specific antibodies, antibody fragments, or can be substituted by aptamers, chemoprobes or other molecules binding the biomarker protein with appropriate specificity and affinity. Biomarker protein levels can further be quantified by iTRAQ or SILAC; by spectral counting or by targeted biomarker protein quantitation using multiple-reaction monitoring (MRM) mass spectrometry.

In the methods of the present invention, the level of said one or more of the markers S100A12, VCAN, ITGAM, LY96, ANXA2, CD86, GPR65, CRISP9, LYZ and VIM is the expression level.

Preferably, the expression level is the mRNA expression level. Methods for detecting mRNA expression level can preferably include but are not limited to PCR, gene expression analyses, microarray analyses, gene expression chip analyses, Whole Transcriptome Sequencing (RNAseq), nanopore sequencing, digital gene expression, hybridization techniques and chromatography as well as any other techniques known in the art, e.g. those described in Ralph Rapley, “The Nucleic Acid Protocols Handbook”, published 2000, ISBN: 978-0-89603-459-4.

The PCR may be quantitative PCR or RealTime PCR, preferably quantitative RealTime PCR (qPCR).

The protein expression level can be detected preferably by immune assays which include the recognition of the protein or protein complex by anti antibody or antibody fragment, comprising but not limited to enzyme linked immunosorbent assays (ELISA), “sandwich” immunoassays, immunoradiometric assays, in situ immunoassays, alphaLISA immunoassays, protein proximity assays, proximity ligation assay technology (e.g. protein qPCR), western blot analysis, immunoprecipitation assays, immunofluorescent assays, flow cytometry, immunohistochemistry (IHC), immuneeletrophoresis, protein immunestaining, confocal microscopy; or by similar methods in which the antibody or antibody fragment is substituted by a chemical probe, aptamer, receptor, interacting protein or other by another biomolecule recognizing the biomarker protein in a specific manner; or by Förster/fluorescence resonance energy transfer (FRET), differential scanning fluorimetry (DSF), microfluidics, spectrophotometry, mass spectrometry, enzymatic assays, surface plasmon resonance, or combinations thereof. Immunoassays may be homogeneous assays or heterogeneous assays. In a homogeneous assay the immunological reaction usually involves the specific antibody, a labeled analyte, and the sample of interest. The signal arising from the label is modified, directly or indirectly, upon the binding of the antibody to the labeled analyte. Both the immunological reaction and detection of the extent thereof can be carried out in a homogeneous solution. Immunochemical labels which may be employed include free radicals, radioisotopes, fluorescent dyes, enzymes, bacteriophages, or coenzymes. In a heterogeneous assay approach, the reagents are usually the sample, the antibody, and means for producing a detectable signal. The antibody can be immobilized on a support, such as a bead, plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels.

In the methods according to the invention, an antibody to the biomarker of interest can be used. In the methods according to the present invention, a kit for detection can be used. Such antibodies and kits are available from commercial sources such as EMD Millipore, R&D Systems for biochemical assays, Thermo Scientific Pierce Antibodies, Novus Biologicals, Aviva Systems Biology, Abnova Corporation, AbD Serotec or others. Alternatively, antibodies can also be synthesized by any known method. The term “antibody” as used herein is intended to include monoclonal antibodies, polyclonal antibodies, and chimeric antibodies. Antibodies can be conjugated to a suitable solid support (e.g., beads such as protein A or protein G agarose, microspheres, plates, slides or wells formed from materials such as latex or polystyrene) in accordance with known techniques, such as passive binding. Antibodies as described herein may likewise be conjugated to detectable labels or groups such as radiolabels (e.g., ³⁵S), enzyme labels (e.g., horseradish peroxidase, alkaline phosphatase), fluorescent labels (e.g., fluorescein, Alexa, green fluorescent protein, rhodamine), can generated by release of singlet oxygen by phthalocyanine containing beads after irradiation at 680 nM and subsequent absorption and emission of light by acceptor beads containing Europium or Therbium, and oligonucleotide labels. Labels can generate signal directly or indirectly. Signal generated can include fluorescence, radioactivity, luminescence, in accordance with known techniques.

The expression level can be normalized to the expression level of an endogenous gene. An endogenous gene must meet a series of criteria, as known by those skilled in the art, e.g. its expression level must be unaffected by experimental factors, show minimal variability in its expression between tissues and physiological states, etc. Examples of suitable endogenous genes are, e.g, GADPH or HPRT1.

In the methods of the present invention, the evaluation of the morphological blast differentiation and blast counts can be performed in accordance with methods known in the art, for example in accordance to ICSH guidelines (ICSH guidelines for the standardization of bone marrow specimens and reports, Lee S H, Ether W N, Porwit A, Tomonaga M, Peterson L C; International Council for Standardization In Hematology, International journal of laboratory hematology 2008 October; 30(5):349-64) by microscopic examination of smears of bone marrow aspirate and/or peripheral blood stained with the May-Grünwald-Giemsa method or similar Romanofsky staining methods. (Immuno)histochemistry and functional techniques (e.g. chemotaxis/phagocytic test) can be also used for blast identification.

The term “treatment with an LSD1 inhbitor” can comprise or be administration of the LSD1 inhibitor to a subject suffering from leukemia. A non-limiting treatment with an LSD1 inhbitor” can comprise or be administering the LSD1 inhibitor (e.g. ORY-1001) according to the following schedule: 140 microgram/m2/day on a dosing scheme 5 days on, 2 days off, up to 4 cycles.

If the level of one or more of the markers S100A12, VCAN, ITGAM, LY96, ANXA2, CD86, GPR65, CRISP9, LYZ and VIM is not increased in a sample of the subject compared to a control, the treatment with said LSD1 inhibitor can be adapted (e.g. the exemplary treatment specified above can be adapted).

For example, said adaption of the treatment with said LSD1 inhibitor can comprise or be termination of the treatment with said LSD1 inhibitor.

For example, said adaption of the treatment with said LSD1 inhibitor comprises increasing the dose of said LSD1 inhibitor. The dose can, for example, be increased until a response to said LSD1 inhibitor can be determined (e.g.

either by determining an increase level of one of the markers to be used herein and/or by determining a (clinical) reponse, such as a decreased number/percentage of blasts and/or an increased number/percentage of differentiated blasts). The dose can be further (continuously) increased until a plateau is reached, e.g. until the level of one of the markers to be used herein does not further increase and/or until the number/percentage of blasts does not further decrease and/or an number/percentage of differentiated blasts does not further increase; or until a maximum desirable level of marker induction is reached.

If the level of only one marker in a sample from a subject is not increased and the level of all remaining markers is increased, this would not be necessarily interpreted as meaning that the dose given was too low and needs to be adapted/increased. Thus, if none of the markers is increased compared to the control (or if only 1 marker, or if only 2 markers are above said cutoff), then this might suggest there is not enough LSD1 inhibition (target engagement) to lead to a clinical response in said subject. In such a situation the adaption of the treatment can be contemplated, particularly an increase of the dose of the LSD1 inhibitor to be administered to said subject.

It is preferred herein that the method(s) herein above is an in vitro method. “In vitro”, as used herein, means that the method(s) of the invention is (are) are not performed in vivo, i.e. directly on a subject, but on a sample obtained from and separated/isolated from said subject (i.e. removed from its in vivo location).

The LSD1 inhibitor to be used in the methods of the invention can be any LSD1 inhibitor known in the art. As used herein, an LSD1 inhibitor (LSD1i) is a compound which inhibits LSD1. Both irreversible and reversible LSD1i have been reported. Irreversible LSD1 inhibitors exert their inhibitory activity by becoming covalently bound to the FAD cofactor within the LSD1 active site and are generally based on a 2-cyclyl-cyclopropylamino moiety such as a 2-(hetero)arylcyclopropylamino moiety. Reversible inhibitors of LSD1 have also been reported.

LSD1 inhibitors are for example disclosed in: WO2010/043721, WO2010/084160, WO2011/035941, WO2011/042217, WO2011/131697, WO2012/013727, WO2012/013728, WO2012/045883, WO2013/057320, WO2013/057322, WO2010/143582, US2010-0324147, WO2011/022489, WO2011/131576, WO2012/034116, WO2012/135113, WO2013/022047, WO2013/025805, WO2014/058071, WO2014/084298, WO2014/086790, WO2014/164867, WO2014/205213, WO2015/021128, WO2015/031564, US2015-0065434, WO2007/021839, WO2008/127734, WO2015/089192, CN104119280, CN103961340, CN103893163, CN103319466, CN103054869, WO2014/194280, WO2015/089192, WO2015/120281, WO2015/123465, WO2015/123437, WO2015/123424, WO2015/123408, WO2015/134973, WO2015/156417, WO2015/168466, WO2015/181380, WO2015/200843, WO2016/003917, WO2016/004105, WO2016/007722, WO2016/007727, WO2016/007731, WO2016/007736, WO2016/034946, WO2016/037005, WO2016/123387, WO2016/130952, WO2016/161282, WO2016/172496, as well as in K Taeko et al, Bioorg Med Chem Lett. 2015, 25(9):1925-8. doi: 10.1016/j.bmc1.2015.03.030. Epub 2015 Mar 20, PMID: 25827526; S Valente et al, Eur J Med Chem. 2015, 94:163-74. doi: 10.1016/j.ejmech.2015.02.060. Epub 2015 Mar. 3, PMID:25768700; MN Ahmed Khan et al Med. Chem. Commun., 2015, 6, 407-412, DOI: 10.1039/C4MD00330F epub 29 Sep. 2014; M Pieroni et al, Eur J Med Chem. 2015; 92:377-386. doi: 10.1016/j.ejmech.2014.12.032. Epub 2015 Jan. 7. PMID:25585008; V Rodriguez et al, Med. Chem. Commun., 2015, 6, 665-670 DOI: 10.1039/C4MD00507D, Epub 23 Dec. 2014; P Vianello et al, Eur J Med Chem. 2014, 86:352-63. doi: 10.1016/j.ejmech.2014.08.068. Epub 2014 Aug. 27; D P Mould et al, Med. Res. Rev., 2015, 35:586-618. doi:10.1002/med.21334, epub 24 Nov. 2014; L Y Ma et al, 2015, 58(4):1705-16. doi: 10.1021/acs.jmedchem.5b00037. Epub 2015 Feb. 6; S L Nowotarski et al, 2015, 23(7):1601-12. doi: 10.1016/j.bmc.2015.01.049. Epub 2015 Feb. 7. PMID:25725609; C J Kutz et al Med chem comm. 2014, 5(12):1863-1870 PMID: 25580204; C Zhou et al, Chemical Biology & Drug Design,2015, 85(6):659-671. doi:10.1111/cbdd.12461, epub 22 Dec. 2014; P Prusevich et al, ACS Chem Biol. 2014, 9(6):1284-93. doi: 10.1021/0500018s. Epub 2014 Apr. 7; B Dulla et al, Org Biomol Chem 2013, 11, 3103-3107, doi: 10.1039/c3ob40217g; J R Hitchin et al, Med Chem Commun, 2013, 4, 1513-1522 DOI: 10.1039/c3md00226h; and Y Zhou et al, Biorg Med Chem Lett, 2015, online publication 20 Jun. 2015, doi:10.1016/j.bmcl.2015.06.054. LSD1 inhibitors are further disclosed e.g. in WO2017/027678, CN106045862, WO2017/004519, WO2014/164867, WO2017/079476, WO2017/079670, WO2017/090756, WO2017/109061, WO2017/116558, WO2017/114497, CN106432248, CN106478639, CN106831489, CN106928235, CN107033148, WO2017149463, CN107174584, CN107176927, WO2017157322, US20170283397, and JP2017178811.

The LSD1 inhibitor to be used herein is preferably a 2-(hetero)arylcyclopropylamino compound. As used herein, a “2-(hetero)arylcyclopropylamino LSD1i” or a “2-(hetero)arylcyclopropylamino compound” means a LSD1i whose chemical structure comprises a cyclopropyl ring substituted at position 1 with an amino group, which can be optionally substituted, and substituted at position 2 with an aryl or heteroaryl group (wherein the aryl or heteroaryl group can be optionally substituted). Such 2-(hetero)arylcyclopropylamino-based LSD1i are for example disclosed in WO2010/043721, WO2010/084160, WO2011/035941, WO2011/042217, WO2011/131697, WO2012/013727, WO2012/013728, WO2012/045883, WO2013/057320, WO2013/057322, WO2012/135113, WO2013/022047, WO2014/058071, WO2010/143582, US2010-0324147, WO2011/131576, WO2014/084298, WO2014/086790, WO2014/164867, WO2014/194280, WO2015/021128, WO2015/123465, WO2015/123437, WO2015/123424, WO2015/123408, WO2015/156417, WO2015/181380, WO2016/123387 and WO2016/130952. The following compounds are examples of 2-(hetero)arylcyclopropylamino-based LSD1 inhibitors:

4-((4-((((1R,2S)-2-phenylcyclopropyl)amino)methyl)piperidin-1-yl)methyl)benzoic acid;

1-((4-(methoxymethyl)-4-(((1R,2S)-2-phenylcyclopropylamino)methyl)piperidin-1-yl)methyl)cyclobutanecarboxylic acid;

N-[4-[2-[(cyclopropylmethylamino)methyl]cyclopropyl]phenyl]-1-methyl-pyrazole-4-carboxamide;

N-[(2S)-5-{[(1R,2S)-2-(4-fluorophenyl)cyclopropyl]amino}-1-(4-methylpiperazin-1-yl)-1-oxopentan-2-yl]-4-(1H-1, 2,3-triazol-1-yl)benzamide;

4-[2-(4-amino-piperidin-1-yl)-5-(3-fluoro-4-methoxy-phenyl)-1-methyl-6-oxo-1,6-dihydro-pyrimidin-4-yl]-2-fluorobenzonitrile;

and pharmaceutically acceptable salts or solvates thereof.

More preferably, the LSD1 inhibitor is (trans)-N1-((1R,2S)-2-phenylcyclopropyl)cyclohexane-1,4-diamine or a pharmaceutically acceptable salt or solvate thereof. Even more preferably, the LSD1 inhibitor is (trans)-N1-((1R,2S)-2-phenylcyclopropyl)cyclohexane-1,4-diamine bis-hydrochloride. The compound (trans)-N1-((1R,2S)-2-phenylcyclopropyl)cyclohexane-1,4-diamine is also known as ORY-1001 and has been disclosed for example in WO2013/057322, see example 5. Pharmaceutical formulations comprising ORY-1001 for administration to subjects can be prepared following methods known to those skilled in the art, for example as described in WO2013/057322.

Furthermore, therapeutic uses are contemplated, i.e. treatment of the herein identified responders/responding subjects with an LSD1 inhibitor.

For example, the present invention relates to a method of treating a subject suffering from leukemia with an LSD1 inhibitor, wherein the subject is identified as a responder to treatment with an LSD1 inhibitor in accordance with this invention.

The present invention also relates to to an LSD1 inhibitor for use in treating a subject suffering from leukemia, wherein the subject is identified as a responder to treatment with an LSD1 inhibitor in accordance with this invention.

Moreover, kits for use in the invention are provided. For example, a kit for use in carrying out the method in accordance with this invention is provided, comprising means for determining the level of one or more of the markers S100A12, VCAN, ITGAM, LY96, ANXA2, CD86, GPR65, CRISP9, LYZ and VIM.

In a further aspect, the present invention relates to a method for monitoring the response to treatment with an LSD1 inhibitor in a subject suffering from leukemia, said method comprising determining the level of one or more of the markers S100A12, VCAN, ITGAM, LY96, ANXA2, CD86, GPR65, CRISP9, LYZ and VIM, in a sample from said subject, wherein a decreased level of one or more of the markers S100A12, VCAN, ITGAM, LY96, ANXA2, CD86, GPR65, CRISP9, LYZ and VIM compared to a control is indicative for a non-response to treatment.

In one aspect, the present invention relates to a method for the identification of a non-responding subject to treatment with an LSD1 inhibitor, said method comprising determining the level of one or more of the markers S100A12, VCAN, ITGAM, LY96, ANXA2, CD86, GPR65, CRISP9, LYZ and VIM in a sample from a subject suffering from leukemia, wherein a decreased level of one or more of the markers S100A12, VCAN, ITGAM, LY96, ANXA2, CD86, GPR65, CRISP9, LYZ and VIM compared to a control is indicative for a non-responding subject.

In one aspect, the present invention relates to a method of determining whether a proliferative diseased cell is non-responsive to treatment with an LSD1 inhibitor, said method comprising determining the level of one or more of the markers S100A12, VCAN, ITGAM, LY96, ANXA2, CD86, GPR65, CRISP9, LYZ and VIM in a sample from a subject suffering from leukemia, wherein a decreased level of one or more of the markers S100A12, VCAN, ITGAM, LY96, ANXA2, CD86, GPR65, CRISP9, LYZ and VIM compared to a control is indicative for a non-responsive proliferative diseased cell.

For example, if there is no response to treatment with the LSD1 inhibitor, the decision may be taken to discontinue treatment or increase the dose of the LSD1 inhibitor.

The above methods to identify non-responding subjects/diseased cells can comprise determining the level of 2 or more, 3 or more, 4 or more, 5 or more, 6 or more, 7 or more, 8 or more, or 9, or 10 of the markers S100A12, VCAN, ITGAM, LY96, ANXA2, CD86, GPR65, CRISP9, LYZ and VIM. In a more preferred aspect, said methods comprise determining the level of all of the markers S100A12, VCAN, ITGAM, LY96, ANXA2, CD86, GPR65, CRISP9, LYZ and VIM (i.e. of a combination of S100A12, VCAN, ITGAM, LY96, ANXA2, CD86, GPR65, CRISP9, LYZ and VIM). Preferably, said methods comprise determining the level of all of the markers S100A12, VCAN, ITGAM, LY96, ANXA2, CD86, GPR65, CRISP9, LYZ and VIM (i.e. of a combination of S100A12, VCAN, ITGAM, LY96, ANXA2, CD86, GPR65, CRISP9, LYZ and VIM), wherein a subject/diseased cell is identified as non-responsive to treatment if at least 3 of said markers are decreased compared to a control.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of.”

Thus, the terms “comprising”/“including”/“having” mean that any further component (or likewise features, integers, steps and the like) can be present.

The term “consisting of” means that no further component (or likewise features, integers, steps and the like) can be present.

The term “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

Thus, the term “consisting essentially of” means that specific further components (or likewise features, integers, steps and the like) can be present, namely those not materially affecting the essential characteristics of the composition, device or method. In other words, the term “consisting essentially of” (which can be interchangeably used herein with the term “comprising substantially”), allows the presence of other components in the composition, device or method in addition to the mandatory components (or likewise features, integers, steps and the like), provided that the essential characteristics of the device or method are not materially affected by the presence of other components.

The term “method” refers to manners, means, techniques and procedures for accomplishing a given task including, but not limited to, those manners, means, techniques and procedures either known to, or readily developed from known manners, means, techniques and procedures by practitioners of the chemical, biological and biophysical arts.

The present invention is further described by reference to the following non-limiting figures and examples. The Figures show:

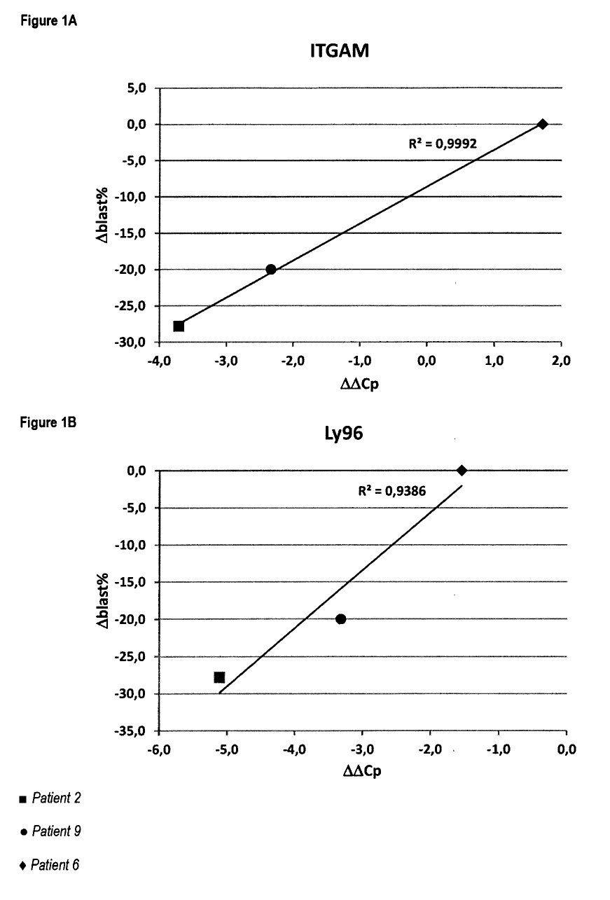

FIG. 1. depicts a correlation between variation of blast counts in bone marrow (as %) versus expression levels (as ΔΔCp) obtained in Example 1 for ITGAM (FIG. 1A) or Ly96 (FIG. 1B), wherein ▪ refers to data for Patient 2, ● for Patient 9 and ♦ for Patient 6.

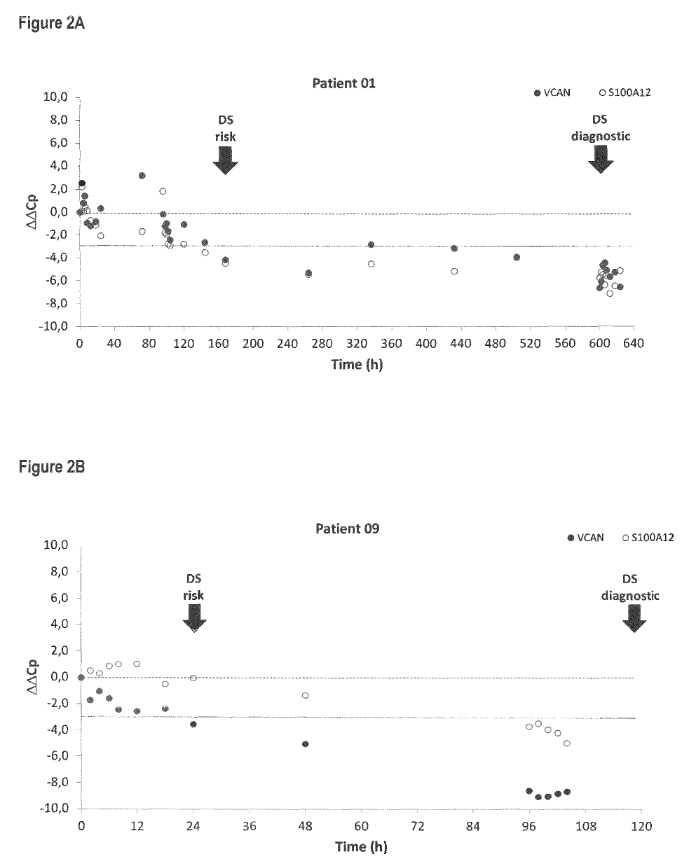

FIG. 2. depicts the evolution of the expression levels (as ΔΔCp) for VCAN and S100A12 over time in patients developing a differentiation syndrome: FIG. 2A: patient 1; FIG. 2B: patient 9

The Example illustrates the invention.

### EXAMPLE 1

### Effect of ORY-1001 on Pharmacodynamic Gene Markers and Correlation with Early Clinical Response in Leukemia Patients 1.1: Patient Population

As part of a Phase I clinical study assessing the human pharmacokinetics and safety of ORY-1001 in acute leukemia patients, an extension cohort of 14 patients (mean age 57; range 30-78, gender 8M/6F) was opened in order to provide a preliminary assessment of efficacy. A summary of the patients recruited in this extension cohort can be found in Table 1.

Preliminary clinical efficacy endpoints included (a) morphological blast differentiation and (b) decrease in blast %. In addition, gene expression determinations of selected markers were performed.

TABLE 1

| Patient characteristics | | n (%) |
|---|---|---|
| FAB subtype | M2 (myeloblastic with maturation) | 3 (21) |
| | M4 (myelomonocytic) | 4 (29) |
| | M5a/M5b (monoblastic/monocytic) | 3 (21) |
| | M6a (erythroleukemic) | 4 (29) |
| | Total | 14 (100) |

1.2: Treatment

Drug: ORY-1001, which is the compound with the following chemical name and structure: (trans)-N1-((1R,2S)-2-phenylcyclopropyl)cyclohexane-1,4-diamine [CAS Reg. No. 1431304-21-0].

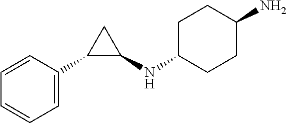 [SMILES: N[C@H]1CC[C@H](N[C@@H]2C[C@H]2c2ccccc2)CC1]

ORY-1001 was administered to patients as the dihydrochloride salt, i.e. (trans)-N1-((1R,2S)-2-phenylcyclopropyl)cyclohexane-1,4-diamine bis-hydrochloride.

Each patient received ORY-1001 for oral intake as a solution at a dose of 140 microgram/m2/day (as free base) q.d., during 5 consecutive days with 2 days of rest, for 4 cycles (total of 28 days), or until disease progression or unacceptable toxicity was observed.

1.3: Clinical Response Determinations

For the evaluation of the morphological blast differentiation and blast counts, smears of bone marrow aspirate and/or peripheral blood were prepared, stained by the May-Grünwald-Giemsa method, and microscopically examined in accordance with ICSH guidelines (ICSH guidelines for the standardization of bone marrow specimens and reports, Lee S H, Erber W N, Porwit A, Tomonaga M, Peterson L C; International Council for Standardization In Hematology, International journal of laboratory hematology 2008 October; 30(5):349-64).

Preliminary evidence of early clinical response was observed in peripheral blood and/or bone marrow, i.e. morphologic blast differentiation and decrease in blast cells, respectively in 5/14 and 6/14 patients throughout all the four FAB subtypes, as shown in Table 2.

TABLE 2

| | FAB | Blast | |
|---|---|---|---|
| Patient No. | subtype | differentiation | % Blast variation^{d} |
| 01^{a} | M4 | Yes | ↓ −55% (peripheral blood) ^{e} |
| 02 | M4 | Yes | ↓ −28% (bone marrow) |
| 03 | M5b | No | N/A |
| 04 | M4 | Yes | =(peripheral blood) |
| 05 | M6a | No | ↓ −53% (bone marrow)^{f} |
| 06 | M4 | No | =(bone marrow) |
| 07 | M6a | No | ↑ 46% (bone marrow) |
| 08 | M6a | No | ↓ −55% (bone marrow) |
| 09 ^{b} | M5a | Yes | ↓ −20% (bone marrow) |
| 10 ^{c} | M5a | No | =(bone marrow) |
| 11 | M2 | Yes | ↑ 58% (bone marrow) |
| 12 | M2 | No | ↓ −32% (peripheral blood) |
| 13 | M6a | No | ↑ 63% (bone marrow) |
| 14 | M2 | No | ↑ 54% (bone marrow) |

^{a}Diagnosed with a differentiation syndrome on day 26
^{b} Diagnosed with a differentiation syndrome on day 6
^{c} Cutaneous leukemia
^{d} % variation of blast % between pre- and post-treatment (day 7 to 29, depending on patient); “=” indicates no or no clinically relevant variation
^{e} Between day 5 and 12 of treatment
^{f}Between day 15 and 29 of treatment
N/A: not available

1.4: Blood Sampling

All patients underwent serial collection of whole blood at pre-established time points up to 768 h (day 33) after the first dose, i.e. pre-dose, and 2, 4, 6, 8, 12, 18, 24, 48, 72, 96, 98, 100, 102, 104, 120, 144, 168, 264, 336, 432, 504, 600, 602, 604, 606, 608, 612, 618, 624, 672, and 768 h post-dose. Five ml of blood were collected using an S-Monovette K3 EDTA tube.

1.5: Sample Processing for RNA Extraction

Plasma for pharmacokinetic determinations was separated by centrifugation. The remaining cell volume was resuspended in 2 mL PBS and an aliquot of 2.5 mL was stabilized in a PAXgene® Blood RNA tube as described by the vendor and kept frozen for subsequent RNA extraction and qRT-PCR.

1.6: RNA Extraction

RNA extraction was performed using PAXgene® Blood RNA Kit (PreAnalytix) as described by the vendor. RNA quality was assessed using an Agilent 2100 Bioanalyzer™ and quantity was measured using a NanoDrop™ spectrophotometer.

1.7: Reverse Transcription

An amount of 0.5 micrograms of total RNA was reverse transcribed to obtain 1st strand cDNA (iScript® Reverse Transcription Supermix for RTqPCR; Bio-Rad).

1.8: Gene Expression Analysis by qRT-PCR

Gene expression was analyzed by qRT-PCR, a variant of the PCR (Polymerase Chain Reaction) method that permits the simultaneous exponential amplification and detection of specific cDNA fragments. Taqman gene expression assays were used, which employ the principle of doubly labeled hydrolysis probes marked with a fluorescent moiety at their 5′ end and with a quencher moiety at the 3′ end, which prevents the generation of fluorescence according to the Förster energy transfer principle.

During the amplification process, the hydrolysis probe hybridizes to its complementary sequence in the target amplicon. During each cycle, the Taq polymerase initiates the production of a copy of the target sequence starting from the primer. When the Taq polymerase reaches the hydrolysis probe, its 5′-3′ exonuclease activity fragments the hydrolysis probe, and liberates the fluorescent group from the quencher moiety, resulting in the emission of a fluorescent signal.

In the exponential phase of the amplification reaction, the intensity of the fluorescence is directly proportional to the quantity of PCR product formed. The LightCycler® 480 Software determines the “crossing point” (Cp), i.e. the point where the reaction's fluorescence reaches the maximum of the second derivative of the amplification curve, which corresponds to the point where the acceleration of the fluorescence signal is at its maximum. The Cp values reflect the target mRNA concentration in the original RNA sample. Differences in Cp values (ΔCp) for a gene X of interest in a given sample relative to a control sample reflect changes in mRNA concentration of the gene X in a given amount of total RNA in the respective sample, and are calculated as:

ΔCp, gene X=Cp(sample, gene X)−Cp(control, gene X)

To compensate for errors in the determination of RNA concentration or efficiency of 1^{st} strand synthesis or amplification, an endogenous reference gene is usually assessed in parallel to the gene X of interest for normalization, and the ΔΔCp is then calculated as:

ΔΔCp, gene X=[Cp(sample, gene X)−Cp(sample, reference gene)]−[Cp(control, gene X)−Cp(control, reference gene)]

The fold change in mRNA concentration is calculated as 2^{-ΔΔCp}, a negative ΔΔCp representing an increase in the expression level, and vice versa.

For a gene to be regarded as a reliable reference, it must meet a series of criteria, as known by those skilled in the art, e.g. its expression level being unaffected by experimental factors, showing minimal variability in its expression between tissues and physiological states, etc. Examples of suitable endogenous genes are GAPDH and HPRT1, among others.

An amount of 0.5 micrograms of the 1st strand product was used to perform qRT-PCR reactions in triplicate (Taqman® gene expression assay, Life technologies, see Table 3) in a Roche LightCycler®480. In order to analyze the changes in the expression levels of ANXA2, CAMSAP2, CD86, CRISP9, CTSG, GPR65, ITGAM, LY96, LYZ, S100A12, VCAN, and VIM, ΔΔCp values for a given patient and time point were calculated as described above, relative to the endogenous reference gene HPRT1 and to a control sample obtained from the same patient at pre-dose (i.e. prior to administration of the first dose of ORY-1001 to said patient).

TABLE 3

| Gene | TaqMan ® Gene Expression Assay |
|---|---|
| ANXA2 | Hs01561520_m1 |
| CAMSAP2 | Hs01115863_m1 |
| CD86 | Hs01567026_m1 |
| CRISP9 | Hs00542137_m1 |
| CTSG | Hs01113415_g1 |
| GPR65 | Hs01097741_s1 |
| ITGAM | Hs00355885_m1 |
| LY96 | Hs01026734_m1 |
| LYZ | Hs00426232_m1 |
| S100A12 | Hs00942835_g1 |
| VCAN | Hs00171642_m1 |
| VIM | Hs00185584_m1 |
| HPRT1 (endogenous reference) | Hs02800695_m1 |

For the analysis of the gene expression data, the time point (or time interval) showing the maximum response is typically selected. This time point/interval may change depending on the specific dose, administration scheme, etc. In the present study, all the gene expression and correlation analysis was performed by using the data obtained after administration of day 5, i.e. within the time interval between 98 and 168 h after the first dose. The maximum response observed within this time interval is referred to in the tables herein as “Maximum response (ΔΔCp) on day 5”. This time interval was selected based on the fact that gene expression levels were overall qualitatively comparable to the maximum response achieved at the end of treatment (i.e. after administration on day 26) (see Table 4 as an example, a comparison of maximum response on days 1, 5, and 26 for 2 patients and genes).

TABLE 4

| GPR65 | | | | S100A12 | | |
|---|---|---|---|---|---|---|
| | Day 1 ^{a} | Day 5 ^{b} | Day 26 ^{c} | Day 1 ^{a} | Day 5 ^{b} | Day 26 ^{c} |
| Patient 01 | 2.2 | −1.8 | −3.2 | −2.1 | −4.5 | −7.1 |
| Patient 02 | −1.1 | −1.4 | −4.8 | −0.4 | −1.7 | −2.8 |

^{a} Maximum response (in ΔΔCp) within first 24 h after the first administration.
^{b} Maximum response (in ΔΔCp) within 98 and 168 h after the first administration.
^{c} Maximum response (in ΔΔCp) within 602 and 768 h after the first administration.

In total, expression changes in 12 potential PD marker genes associated to blast differentiation (mostly to monocyte/macrophage) were monitored in peripheral blood of all 14 patients. Results of maximum response on day 5 are shown in Table 5.

TABLE 5

| Patient | Maximum response (ΔΔCp) on day 5 | | | | | | | | | | | |
|---|---|---|---|---|---|---|---|---|---|---|---|---|
| No. | VCAN | LYZ | GPR65 | S100A12 | Ly96 | CTSG | ANXA2 | CRISP9 | VIM | CAMSAP2 | CD86 | ITGAM |
| 01 | −4.2 | −3.9 | −1.8 | −4.5 | −3.2 | −3.6 | −2.6 | −3.5 | −2.2 | −2.3 | −4.4 | −3.0 |
| 02 | −1.2 | −0.6 | −1.4 | −1.7 | −5.1 | −1.7 | −1.4 | −2.8 | −0.4 | −3.0 | −2.5 | −3.7 |
| 03 | 1.6 | nm | nm | nm | −1.6 | nm | nm | nm | nm | nm | nm | nm |
| 04 | −1.2 | 2.4 | 4.4 | 3.9 | −4.1 | −2.5 | 3.2 | 2.5 | 1.3 | −2.9 | −2.8 | −2.8 |
| 05 | 1.2 | 1.2 | −1.5 | −2.1 | −1.5 | −0.7 | −1.4 | −1.1 | −0.6 | −1.4 | 1.3 | 1.8 |
| 06 | 2.2 | 2.9 | −1.3 | 1.7 | −1.6 | nm | nm | nm | nm | nm | −1.8 | 1.7 |
| 07 | 2.0 | 3.2 | −3.8 | 1.3 | −2.9 | 2.3 | 2.0 | −2.9 | −1.2 | −2.8 | 2.2 | 1.6 |
| 08 | −0.8 | 3.2 | 3.1 | 2.7 | 2.3 | 3.9 | −1.1 | 1.2 | −1.3 | −1.9 | 2.5 | 2.1 |
| 09 | −9.1 | −1.2 | −0.9 | −5.0 | −3.3 | 3.3 | −2.6 | −3.5 | −0.5 | 1.2 | −2.9 | −2.3 |
| 10 | 1.8 | 1.7 | 2.3 | 3.0 | 3.0 | 1.8 | 1.9 | 2.3 | 2.2 | 3.1 | 2.2 | 3.0 |
| 11 | 3.4 | 2.9 | −4.8 | 0.8 | −4.3 | 2.2 | −1.9 | 2.2 | 1.7 | 1.6 | −3.0 | 3.1 |
| 12 | −2.2 | −2.3 | −3.0 | 2.4 | −3.1 | −1.8 | −2.0 | −3.5 | −2.4 | −3.7 | −2.0 | −1.3 |
| 13 | 3.4 | 2.5 | −3.8 | 2.0 | −4.8 | 7.6 | −3.8 | 3.1 | −3.4 | −4.3 | −3.3 | −2.3 |
| 14 | −2.1 | −2.2 | −2.3 | 2.1 | 2.4 | nm | −1.6 | −3.1 | nm | −2.5 | 0.8 | 3.2 |

nm: not measured due to limited sample amount

1.9: Correlations Between Gene Expression and Response to Treatment with an LSD1 Inhibitor

Possible correlations between expression changes in marker genes described in table 5 and response to ORY-1001 treatment (see Table 2) were investigated.

A 1.3 to 550-fold (corresponding to −0.4 to −9.1 ΔΔCp) up-regulation of the gene markers S100A12, VCAN, ITGAM, LY96, ANXA2, CD86, GPR65, CRISP9, LYZ, and VIM was observed in patients showing both blast morphological differentiation and a decrease in blast cells, particularly in M4/M5 subtypes (see Table 6). In contrast, some of the genes were down-regulated (0.6 to 0.05-fold change, corresponding to 0.8 to 4.4 ΔΔCp) in patients showing no morphological differentiation and/or no effect or increase in blast cells (see Table 7). LYZ, GPR65, ANXA2, S100A12, CRISP9, and VIM were clearly differentially regulated in M4/M5 patients showing blast count decrease (markers up-regulated) compared to those showing blast differentiation with no decrease in blast count (markers down-regulated). The expression levels of Ly96 and ITGAM did additionally correlate with the variation of blast cells in bone marrow (see FIGS. 1A and 1B), particularly in M4/M5 subtypes, further supporting the utility of these marker genes in monitoring response to ORY-1001 treatment in easily accessible samples such as peripheral blood. On the other hand, CTSG and CAMSAP2 were not considered suitable for this purpose, as they both showed a non-consistent response, i.e. down-regulation in patients showing a blast decrease and/or morphological differentiation, and up-regulation in patients showing an increase in blast counts.

TABLE 6

| Patient | Blast | % blast | Maximum response (ΔΔCp) on day 5 | | | | | | | | | | | |
|---|---|---|---|---|---|---|---|---|---|---|---|---|---|---|
| No. | differentiation | variation | VCAN | LYZ | GPR65 | S100A12 | Ly96 | CTSG | ANXA2 | CRISP9 | VIM | CAMSAP2 | CD86 | ITGAM |
| 01 | Yes | ↓ −55% | −4.2 | −3.9 | −1.8 | −4.5 | −3.2 | −3.6 | −2.6 | −3.5 | −2.2 | −2.3 | −4.4 | −3.0 |
| 02 | Yes | ↓ −28% | −1.2 | −0.6 | −1.4 | −1.7 | −5.1 | −1.7 | −1.4 | −2.8 | −0.4 | −3.0 | −2.5 | −3.7 |
| 09 | Yes | ↓ −20% | −9.1 | −1.2 | −0.9 | −5.0 | −3.3 | 3.3 | −2.6 | −3.5 | −0.5 | 1.2 | −2.9 | −2.3 |

TABLE 7

| Patient | Blast | % blast | Maximum response (ΔΔCp) on day 5 | | | | | | | | | | | |
|---|---|---|---|---|---|---|---|---|---|---|---|---|---|---|
| No. | differentiation | variation | VCAN | LYZ | GPR65 | S100A12 | Ly96 | CTSG | ANXA2 | CRISP9 | VIM | CAMSAP2 | CD86 | ITGAM |
| 03 | No | na | 1.6 | nm | nm | nm | −1.6 | nm | nm | nm | nm | nm | nm | nm |
| 04 | Yes | = | −1.2 | 2.4 | 4.4 | 3.9 | −4.1 | −2.5 | 3.2 | 2.5 | 1.3 | −2.9 | −2.8 | −2.8 |
| 06 | No | = | 2.2 | 2.9 | −1.3 | 1.7 | −1.6 | nm | nm | nm | nm | nm | −1.8 | 1.7 |
| 14 | No | ↓ 54% | −2.1 | −2.2 | −2.3 | 2.1 | 2.4 | nm | −1.6 | −3.1 | nm | −2.5 | 0.8 | 3.2 |

na: not available;
nm: not measured due to limited sample amount

1.10: Correlation between S100A12 and VCAN Gene Expression and Differentiation Syndrome

The differentiation syndrome (DS), also known as retinoic acid syndrome, is a relatively common and potentially severe complication seen in AML patients treated with differentiating agents, such as all-trans retinoic acid and/or arsenic trioxide. The differentiation of vast numbers of leukemic blasts may lead to cellular migration, endothelial activation, and release of interleukins and vascular factors responsible for tissue damage, finally developing in a syndrome characterized by unexplained fever, acute respiratory distress with interstitial pulmonary infiltrates, and/or a vascular capillary leak leading to acute renal failure.

S100A12 and VCAN showed an exacerbated (18 to 550-fold, corresponding to −4.2 to −9.1 ΔΔCp) up-regulation pattern in patients developing a differentiation syndrome (Patients 01 and 09, see Table 5) within 98 and 168 h after the first dose, and this could be already observed up to 2 weeks prior to its clinical diagnosis (see FIGS. 2A and 2B). These two markers may thus be a useful tool for early monitoring the risk of developing a differentiation syndrome in AML patients receiving treatment with an LSD1 inhibitor (e.g. ORY-1001), particularly in M4/M5 subtypes.

The present invention refers to the following nucleotide and amino acid sequences:

The sequences provided herein are available in the NCBI database and can be retrieved from www.ncbi.nlm.nih.qov/sites/entrez?db=gene; Theses sequences also relate to annotated and modified sequences. The present invention also provides techniques and methods wherein homologous sequences, and variants of the concise sequences provided herein are used. Preferably, such “variants” are genetic variants, e.g. splice variants.

Exemplary amino acid sequences and nucleotide sequences of human S100A12, VCAN, ITGAM, LY96, ANXA2, CD86, GPR65, CRISP9, LYZ, VIM, CAMSAP2, CTSG, Gapdh, and Hprt1 are shown in SEQ ID NO: 1 to 28 herein below.

```
SEQ ID No. 1: Nucleotide sequence encoding Homo sapiens S100 calcium binding protein
A12 (S100A12),
mRNA NCBI Reference Sequence: NM_005621.1. The coding region ranges from nucleotide
60 to nucleotide 347 (highlighted in bold). It is understood that the mRNA
corresponds to the sequence below (i.e. is identical to that sequence) with the
exception that the “t” (thymidine) residue is replaced by a “uracil” (u) residue.
ORIGIN
    1 accactgctg gctttttgct gtagctccac attcctgtgc attgaggggt taacattagg
  61 ctgggaagat gacaaaactt gaagagcatc tggagggaat tgtcaatatc ttccaccaat
121 actcagttcg gaaggggcat tttgacaccc tctctaaggg tgagctgaag cagctgctta
181 caaaggagct tgcaaacacc atcaagaata tcaaagataa agctgtcatt gatgaaatat
241 tccaaggcct ggatgctaat caagatgaac aggtcgactt tcaagaattc atatccctgg
301 tagccattgc gctgaaggct gcccattacc acacccacaa agagtaggta gctctctgaa
361 ggctttttac ccagcaatgt cctcaatgag ggtcttttct ttccctcacc aaaacccagc
421 cttgcccgtg gggagtaaga gttaataaac acactcacga aaagtt //
SEQ ID No. 2: Amino acid sequence of Homo sapiens S100 calcium binding protein
A12 (S100A12), protein
UniProtKB/Swiss-Prot: S10AC_HUMAN, P80511
MTKLEEHLEGIVNIFHQYSVRKGHFDTLSKGELKQLLTKELANTIKNIKDKAVIDEIFQGLDANQDEQVDFQEFISLVIAIALK
AAHYHTHKE
SEQ ID No. 3: Nucleotide sequence encoding Homo sapiens Versican (VCAN), mRNA
NCBI Reference Sequence: NM_001126336.2. The coding region ranges from nucleotide
357 to nucleotide 2324 (highlighted in bold). It is understood that the mRNA
corresponds to the sequence below (i.e. is identical to that sequence) with the
exception that the “t” (thymidine) residue is replaced by a “uracil” (u) residue.
ORIGIN
            1 cttcttctcg ctgagtctcc tcctcggctc tgacggtaca gtgatataat gatgatgggt
          61 gtcacaaccc gcatttgaac ttgcaggcga gctgccccga gcctttctgg ggaagaactc
        121 caggcgtgcg gacgcaacag ccgagaacat taggtgttgt ggacaggagc tgggaccaag
        181 atcttcggcc agccccgcat cctcccgcat cttccagcac cgtcccgcac cctccgcatc
        241 cttccccggg ccaccacgct tcctatgtga cccgcctggg caacgccgaa cccagtcgcg
        301 cagcgctgca gtgaattttc cccccaaact gcaataagcc gccttccaag gccaagatgt
        361 tcataaatat aaagagcatc ttatggatgt gttcaacctt aatagtaacc catgcgctac
        421 ataaagtcaa agtgggaaaa agcccaccgg tgaggggctc cctctctgga aaagtcagcc
        481 taccttgtca tttttcaacg atgcctactt tgccacccag ttacaacacc agtgaatttc
        541 tccgcatcaa atggtctaag attgaagtgg acaaaaatgg aaaagatttg aaagagacta
        601 ctgtccttgt ggcccaaaat ggaaatatca agattggtca ggactacaaa gggagagtgt
        661 ctgtgcccac acatcccgag gctgtgggcg atgcctccct cactgtggtc aagctgctgg
        721 caagtgatgc gggtctttac cgctgtgacg tcatgtacgg gattgaagac acacaagaca
        781 cggtgtcact gactgtggat ggggttgtgt ttcactacag ggcggcaacc agcaggtaca
        841 cactgaattt tgaggctgct cagaaggctt gtttggacgt tggggcagtc atagcaactc
        901 cagagcagct ctttgctgcc tatgaagatg gatttgagca gtgtgacgca ggctggctgg
        961 ctgatcagac tgtcagatat cccatccggg ctcccagagt aggctgttat ggagataaga
      1021 tgggaaaggc aggagtcagg acttatggat tccgttctcc ccaggaaact tacgatgtgt
      1081 attgttatgt ggatcatctg gatggtgatg tgttccacct cactgtcccc agtaaattca
      1141 ccttcgagga ggctgcaaaa gagtgtgaaa accaggatgc caggctggca acagtggggg
      1201 aactccaggc ggcatggagg aacggctttg accagtgcga ttacgggtgg ctgtcggatg
      1261 ccagcgtgcg ccaccctgtg actgtggcca gggcccagtg tggaggtggt ctacttgggg
      1321 tgagaaccct gtatcgtttt gagaaccaga caggcttccc tccccctgat agcagatttg
      1381 atgcctactg ctttaaacga cctgatcgct gcaaaatgaa cccgtgcctt aacggaggca
      1441 cctgttatcc tactgaaact tcctacgtat gcacctgtgt gccaggatac agcggagacc
      1501 agtgtgaact tgattttgat gaatgtcact ctaatccctg tcgtaatgga gccacttgtg
      1561 ttgatggttt taacacattc aggtgcctct gccttccaag ttatgttggt gcactttgtg
      1621 agcaagatac cgagacatgt gactatggct ggcacaaatt ccaagggcag tgctacaaat
      1681 actttgccca tcgacgcaca tgggatgcag ctgaacggga atgccgtctg cagggtgccc
      1741 atctcacaag catcctgtct cacgaagaac aaatgtttgt taatcgtgtg ggccatgatt
      1801 atcagtggat aggcctcaat gacaagatgt ttgagcatga cttccgttgg actgatggca
      1861 gcacactgca atacgagaat tggagaccca accagccaga cagcttcttt tctgctggag
      1921 aagactgtgt tgtaatcatt tggcatgaga atggccagtg gaatgatgtt ccctgcaatt
      1981 accatctcac ctatacgtgc aagaaaggaa cagtcgcttg cggccagccc cctgttgtag
      2041 aaaatgccaa gacctttgga aagatgaaac ctcgttatga aatcaactcc ctgattagat
      2101 accactgcaa agatggtttc attcaacgtc accttccaac tatccggtgc ttaggaaatg
      2161 gaagatgggc tatacctaaa attacctgca tgaacccatc tgcataccaa aggacttatt
      2221 ctatgaaata ctttaaaaat tcctcatcag caaaggacaa ttcaataaat acatccaaac
      2281 atgatcatcg ttggagccgg aggtggcagg agtcgaggcg ctgatcccta aaatggcgaa
      2341 catgtgtttt catcatttca gccaaagtcc taacttcctg tgcctttcct atcacctcga
      2401 gaagtaatta tcagttggtt tggatttttg gaccaccgtt cagtcatttt gggttgccgt
      2461 gctcccaaaa cattttaaat gaaagtattg gcattcaaaa agacagcaga caaaatgaaa
      2521 gaaaatgaga gcagaaagta agcatttcca gcctatctaa tttctttagt tttctatttg
      2581 cctccagtgc agtccatttc ctaatgtata ccagcctact gtactattta aaatgctcaa
      2641 tttcagcacc gatggccatg taaataagat gatttaatgt tgattttaat cctgtatata
      2701 aaataaaaag tcacaatgag tttgggcata tttaatgatg attatggagc cttagaggtc
      2761 tttaatcatt ggttcggctg cttttatgta gtttaggctg gaaatggttt cacttgctct
      2821 ttgactgtca gcaagactga agatggcttt tcctggacag ctagaaaaca caaaatcttg
      2881 taggtcattg cacctatctc agccataggt gcagtttgct tctacatgat gctaaaggct
      2941 gcgaatggga tcctgatgga actaaggact ccaatgtcga actcttcttt gctgcattcc
      3001 tttttcttca cttacaagaa aggcctgaat ggaggacttt tctgtaacca ggaacatttt
      3061 ttaggggtca aagtgctaat aattaactca accaggtcta ctttttaatg gctttcataa
      3121 cactaactca taaggttacc gatcaatgca tttcatacgg atatagacct agggctctgg
      3181 agggtggggg attgttaaaa cacatgcaaa aaaaaaaaaa aaaaaaaaaa aagaaatttt
      3241 gtatatataa ccattttaat cttttataaa gttttgaatg ttcatgtatg aatgctgcag
      3301 ctgtgaagca tacataaata aatgaagtaa gccatactga tttaatttat tggatgttat
      3361 tttccctaag acctgaaaat gaacatagta tgctagttat ttttcagtgt tagcctttta
      3421 ctttcctcac acaatttgga atcatataat ataggtactt tgtccctgat taaataatgt
      3481 gacggataga atgcatcaag tgtttattat gaaaagagtg gaaaagtata tagcttttag
      3541 caaaaggtgt ttgcccattc taagaaatga gcgaatatat agaaatagtg tgggcatttc
      3601 ttcctgttag gtggagtgta tgtgttgaca tttctcccca tctcttccca ctctgttttc
      3661 tccccattat ttgaataaag tgactgctga agatgacttt gaatccttat ccacttaatt
      3721 taatgtttaa agaaaaacct gtaatggaaa gtaagactcc ttccctaatt tcagtttaga
      3781 gcaacttgaa gaagagtaga caaaaaataa aatgcacata gaaaaagaga aaaagggcac
      3841 aaagggattg gcccaatatt gattcttttt ttataaaacc tcctttggct tagaaggaat
      3901 gactctagct acaataatac acagtatgtt taagcaggtt cccttggttg ttgcattaaa
      3961 tgtaatccac ctttaggtat tttagagcac agaacaacac tgtgttgatc tagtaggttt
      4021 ctatttttcc tttctcttta caatgcacat aatactttcc tgtatttata tcataacgtg
      4081 tatagtgtaa aatgtgaatg actttttttg tgaatgaaaa tctaaaatct ttgtaacttt
      4141 ttatatctgc ttttgtttca ccaaagaaac ctaaaatcct tcttttacta cac //
SEQ ID No. 4: Amino acid sequence of Homo sapiens Versican (VCAN), protein
UniProtKB/Swiss-Prot: CSPG2_HUMAN, P13611
MFINIKSILWMCSTLIVTHALHKVKVGKSPPVRGSLSGKVSLPCHFSTMPTLPPSYNTSEFLRIKWSKIEVDKNGKDLKETTVL
VAQNGNIKIGQDYKGRVSVPTHPEAVGDASLTVVKLLASDAGLYRCDVMYGIEDTQDTVSLTVDGVVFHYRAATSRYTLNFEAA
QKACLDVGAVIATPEQLFAAYEDGFEQCDAGWLADQTVRYPIRAPRVGCYGDKMGKAGVRTYGFRSPQETYDVYCYVDHLDGDV
FHLTVPSKFTFEEAAKECENQDARLATVGELQAAWRNGFDQCDYGWLSDASVRHPVTVARAQCGGGLLGVRTLYRFENQTGFPP
PDSRFDAYVFKRPDRCKMNPCLNGGTCYPTETDYVCTCVPGYSGDQCELDFDECHSNPCRNGATCVDGFNTFRCLCLPSYVGAL
CEQDTETCDYGWHKFQGQCYKYFAHRRTWDAAERECRLQGAHLTSILSHEEQMFVNRVGHDYQWIGLNDKMFEHDFRWTDGSTL
QYENWRPNQPDSFFSAGEDCVVIIWHENGQWNDVPCNYHLTYTCKKGTVACGQPPVVENAKTFGKMKPRYEINSLIRYHCKDGF
IQRHLPTIRCLGNGRWAIPKITCMNPSAYQRTSMKYFKNSSSAKDNSINTSKHDHRWSRRWQESRR
SEQ ID No. 5: Nucleotide sequence encoding Homo sapiens Integrin subunit alpha M
(ITGAM), mRNA
NCBI Reference Sequence: NM_001145808.1. The coding region ranges from nucleotide
99 to nucleotide 3560 (highlighted in bold). It is understood that the mRNA
corresponds to the sequence below (i.e. is identical to that sequence) with the
exception that the “t” (thymidine) residue is replaced by a “uracil” (u) residue.
ORIGIN
            1 ttttctgccc ttctttgctt tggtggcttc cttgtggttc ctcagtggtg cctgcaaccc
          61 ctggttcacc tccttccagg ttctggctcc ttccagccat ggctctcaga gtccttgtgt
        121 taacagcctt gaccttatgt catgggttca acttggacac tgaaaacgca atgaccttcc
        181 aagagaacgc aaggggcttc gggcagagcg tggtccagct tcagggatcc agggtggtgg
        241 ttggagcccc ccaggagata gtggctgcca accaaagggg cagcctctac cagtgcgact
        301 acagcacagg ctcatgcgag cccatccgcc tgcaggtccc cgtggaggcc gtgaacatgt
        361 ccctgggcct gtccctggca gccaccacca gcccccctca gctgctggcc tgtggtccca
        421 ccgtgcacca gacttgcagt gagaacacgt atgtgaaagg gctctgcttc ctgtttggat
        481 ccaacctacg gcagcagccc cagaagttcc cagaggccct ccgagggtgt cctcaagagg
        541 atagtgacat tgccttcttg attgatggct ctggtagcat catcccacat gactttcggc
        601 ggatgaagga gtttgtctca actgtgatgg agcaattaaa aaagtccaaa accttgttct
        661 ctttgatgca gtactctgaa gaattccgga ttcactttac cttcaaagag ttccagaaca
        721 accctaaccc aagatcactg gtgaagccaa taacgcagct gcttgggcgg acacacacgg
        781 ccacgggcat ccgcaaagtg gtacgagagc tgtttaacat caccaacgga gcccgaaaga
        841 atgcctttaa gatcctagtt gtcatcacgg atggagaaaa gtttggcgat cccttgggat
        901 atgaggatgt catccctgag gcagacagag agggagtcat tcgctacgtc attggggtgg
        961 gagatgcctt ccgcagtgag aaatcccgcc aagagcttaa taccatcgca tccaagccgc
      1021 ctcgtgatca cgtgttccag gtgaataact ttgaggctct gaagaccatt cagaaccagc
      1081 ttcgggagaa gatctttgcg atcgagggta ctcagacagg aagtagcagc tcctttgagc
      1141 atgagatgtc tcaggaaggc ttcagcgctg ccatcacctc taatggcccc ttgctgagca
      1201 ctgtggggag ctatgactgg gctggtggag tctttctata tacatcaaag gagaaaagca
      1261 ccttcatcaa catgaccaga gtggattcag acatgaatga tgcttacttg ggttatgctg
      1321 ccgccatcat cttacggaac cgggtgcaaa gcctggttct gggggcacct cgatatcagc
      1381 acatcggcct ggtagcgatg ttcaggcaga acactggcat gtgggagtcc aacgctaatg
      1441 tcaagggcac ccagatcggc gcctacttcg gggcctccct ctgctccgtg gacgtggaca
      1501 gcaacggcag caccgacctg gtcctcatcg gggcccccca ttactacgag cagacccgag
      1561 ggggccaggt gtccgtgtgc cccttgccca gggggcagag ggctcggtgg cagtgtgatg
      1621 ctgttctcta cggggagcag ggccaaccct ggggccgctt tggggcagcc ctaacagtgc
      1681 tgggggacgt aaatggggac aagctgacgg acgtggccat tggggcccca ggagaggagg
      1741 acaaccgggg tgctgtttac ctgtttcacg gaacctcagg atctggcatc agcccctccc
      1801 atagccagcg gatagcaggc tccaagctct ctcccaggct ccagtatttt ggtcagtcac
      1861 tgagtggggg ccaggacctc acaatggatg gactggtaga cctgactgta ggagcccagg
      1921 ggcacgtgct gctgctcagg tcccagccag tactgagagt caaggcaatc atggagttca
      1981 atcccaggga agtggcaagg aatgtatttg agtgtaatga tcaggtggtg aaaggcaagg
      2041 aagccggaga ggtcagagtc tgcctccatg tccagaagag cacacgggat cggctaagag
      2101 aaggacagat ccagagtgtt gtgacttatg acctggctct ggactccggc cgcccacatt
      2161 cccgcgccgt cttcaatgag acaaagaaca gcacacgcag acagacacag gtcttggggc
      2221 tgacccagac ttgtgagacc ctgaaactac agttgccgaa ttgcatcgag gacccagtga
      2281 gccccattgt gctgcgcctg aacttctctc tggtgggaac gccattgtct gctttcggga
      2341 acctccggcc agtgctggcg gaggatgctc agagactctt cacagccttg tttccctttg
      2401 agaagaattg tggcaatgac aacatctgcc aggatgacct cagcatcacc ttcagtttca
      2461 tgagcctgga ctgcctcgtg gtgggtgggc cccgggagtt caacgtgaca gtgactgtga
      2521 gaaatgatgg tgaggactcc tacaggacac aggtcacctt cttcttcccg cttgacctgt
      2581 cctaccggaa ggtgtccacg ctccagaacc agcgctcaca gcgatcctgg cgcctggcct
      2641 gtgagtctgc ctcctccacc gaagtgtctg gggccttgaa gagcaccagc tgcagcataa
      2701 accaccccat cttcccggaa aactcagagg tcacctttaa tatcacgttt gatgtagact
      2761 ctaaggcttc ccttggaaac aaactgctcc tcaaggccaa tgtgaccagt gagaacaaca
      2821 tgcccagaac caacaaaacc gaattccaac tggagctgcc ggtgaaatat gctgtctaca
      2881 tggtggtcac cagccatggg gtctccacta aatatctcaa cttcacggcc tcagagaata
      2941 ccagtcgggt catgcagcat caatatcagg tcagcaacct ggggcagagg agcctcccca
      3001 tcagcctggt gttcttggtg cccgtccggc tgaaccagac tgtcatatgg gaccgccccc
      3061 aggtcacctt ctccgagaac ctctcgagta cgtgccacac caaggagcgc ttgccctctc
      3121 actccgactt tctggctgag cttcggaagg cccccgtggt gaactgctcc atcgctgtct
      3181 gccagagaat ccagtgtgac atcccgttct ttggcatcca ggaagaattc aatgctaccc
      3241 tcaaaggcaa cctctcgttt gactggtaca tcaagacctc gcataaccac ctcctgatcg
      3301 tgagcacagc tgagatcttg tttaacgatt ccgtgttcac cctgctgccg ggacaggggg
      3361 cgtttgtgag gtcccagacg gagaccaaag tggagccgtt cgaggtcccc aaccccctgc
      3421 cgctcatcgt gggcagctct gtcgggggac tgctgctcct ggccctcatc accgccgcgc
      3481 tgtacaagct cggcttcttc aagcggcaat acaaggacat gatgagtgaa gggggtcccc
      3541 cgggggccga accccagtag cggctccttc ccgacagagc tgcctctcgg tggccagcag
      3601 gactctgccc agaccacacg tagcccccag gctgctggac acgtcggaca gcgaagtatc
      3661 cccgacagga cgggcttggg cttccatttg tgtgtgtgca agtgtgtatg tgcgtgtgtg
      3721 caagtgtctg tgtgcaagtg tgtgcacatg tgtgcgtgtg cgtgcatgtg cacttgcacg
      3781 cccatgtgtg agtgtgtgca agtatgtgag tgtgtccaag tgtgtgtgcg tgtgtccatg
      3841 tgtgtgcaag tgtgtgcatg tgtgcgagtg tgtgcatgtg tgtgctcagg ggcgtgtggc
      3901 tcacgtgtgt gactcagatg tctctggcgt gtgggtaggt gacggcagcg tagcctctcc
      3961 ggcagaaggg aactgcctgg gctcccttgt gcgtgggtga agccgctgct gggttttcct
      4021 ccgggagagg ggacggtcaa tcctgtgggt gaagacagag ggaaacacag cagcttctct
      4081 ccactgaaag aagtgggact tcccgtcgcc tgcgagcctg cggcctgctg gagcctgcgc
      4141 agcttggatg gagactccat gagaagccgt gggtggaacc aggaacctcc tccacaccag
      4201 cgctgatgcc caataaagat gcccactgag gaatgatgaa gcttcctttc tggattcatt
      4261 tattatttca atgtgacttt aattttttgg atggataagc ttgtctatgg tacaaaaatc
      4321 acaaggcatt caagtgtaca gtgaaaagtc tccctttcca gatattcaag tcacctcctt
      4381 aaaggtagtc aagattgtgt tttgaggttt ccttcagaca gattccaggc gatgtgcaag
      4441 tgtatgcacg tgtgcacaca caccacacat acacacacac aagctttttt acacaaatgg
      4501 tagcatactt tatattggtc tgtatcttgc tttttttcac caatatttct cagacatcgg
      4561 ttcatattaa gacataaatt actttttcat tcttttatac cgctgcatag tattccattg
      4621 tgtgagtgta ccataatgta tttaaccagt cttcttttga tatactattt tcattctctt
      4681 gttattgcat caatgctgag ttaataaatc aaatatatgt catttttgca tatatgtaag
      4741 gataa
SEQ ID No. 6: Amino acid sequence of Homo sapiens Integrin subunit alpha M (ITGAM),
protein
UniProtKB/Swiss-Prot: ITAM_HUMAN, P11215
MALRVLLLTATLTCHGFNLDTENAMTFQENARGFGQSVVQLQGSRVVVGAPQEIVAANQRGSLYQCDYSTGSCEPIRLQVPVEA
VNMSLGLSLAATTSPPQLLACGPTVHQTCSENTYVKGLCFLFGSNLRQQPQKFPEALRGCPQEDSDIAFLIDGSGSIIPHDFRR
MKEFVSTVMEQLKKSKTLFSLMQYSEEFRIHFTFKEFQNNPNPRSLVKPITQLLGRTHTATGIRKVVRELFNITNGARKNAFKI
LVVITDGEKFGDPLGYEDVIPEADREGVIRYVIGVGDAFRSEKSRQELNTIASKPPRDHVFQVNNFEALKTIQNQLREKIFAIE
GTQTGSSSSFEHEMSQEGFSAAITSNGPLLSTVGSYDWAGGVFLYTSKEKSTFINMTRVDSDMNDAYLGYAAAIILRNRVQSLV
LGAPRYQHIGLVAMFRQNTGMWESNANVKGTQIGAYFGASLCSVDVDSNGSTDLVLIGAPHYYEQTRGGQVSVCPLPRGRARWQ
CDAVLYGEQGQPWGRFGAALTVLGDVNGDKLTDVAIGAPGEENDRGAVYLFHGTSGSGISPSHSQRIAGSKLSPRLQYFGQSLS
GGQDLTMDGLVDLTVGAQGHVLLLRSQPVLRVKAIMEFNPREVARNVFECNDQVVKGKEAGEVRVCLHVQKSTRDRLREGQIQS
VVTYDLALDSGRPHSRAVFNETKNSTRRQTQVLGLTQTCETLKLQLPNCIEDPVSPIVLRLNFSLVGTPLSAFGNLRPVLAEDA
QRLFTALFPFEKNCGNDNICQDDLSITFSGMSLDCLVVGGPREFNVTVTVRNDGEDSYRTQVTFFFPLDLSYRKVSTLQNQRSQ
RSWRLACESASSTEVSGALKSTSCSINHPIFPENSEVTFNITFDVDSKASLGNKLLLKANVTSENNMPRTNKTEFQLELPVKYA
VYMVVTSHGVSTKYLNFTASENTSRVMQHQYQVSNLGQRSLPISLVFLVPVRLNQTVIWDRPQVTFSENLSSTCHTKERLPSHS
DFLAELRKAPVVNCSIAVCQRIQCDIPFFGIQEEFNATLKGNLSFDWYIKTSHNHLLIVSTAEILFNDSVFTLLPGQGAFVRSQ
TETKVEPFEVPNPLPLIVGSSVGGLLLLALITAALYKLGFFKRQYKDMMSEGGPPGAEPQ
SEQ ID No. 7: Nucleotide sequence encoding Homo sapiens Lymphocyte antigen 96
(Ly96), mRNA
NCBI Reference Sequence: NM_015364.4 The coding region ranges from nucleotide 115
to nucleotide 597 (highlighted in bold). It is understood that the mRNA
corresponds to the sequence below (i.e. is identical to that sequence) with the
exception that the “t” (thymidine) residue is replaced by a “uracil” (u) residue.
ORIGIN
          1 agaaatcatg tgactgatga ctaagttaaa tcttttctgc ttactgaaaa ggaagagtct
        61 gatgattagt tactgatcct ctttgcattt gtaaagcttt ggagatattg aatcatgtta
      121 ccatttctgt ttttttccac cctgttttct tccatattta ctgaagctca gaagcagtat
      181 tgggtctgca actcatccga tgcaagtatt tcatacacct actgtgataa aatgcaatac
      241 ccaatttcaa ttaatgttaa cccctgtata gaattgaaag gatccaaagg attattgcac
      301 attttctaca ttccaaggag agatttaaag caattatatt tcaatctcta tataactgta
      361 aacaccatga atcttccaaa gcgcaaagaa gttatttgcc gaggatctga tgacgattac
      421 tctttttgca gagctctgaa gggagagact gtgaatacaa caatatcatt ctccttcaag
      481 ggaataaaat tttctaaggg aaaatacaaa tgtgttgttg aagctatttc tgggagccca
      541 gaagaaatgc tcttttgctt ggagtttgtc atcctacacc aacctaattc aaattagaat
      601 aaattgagta tttaaaaaaa aaaaaaaaaa aaaaaaaaaa aa
//
SEQ ID No. 8: Amino acid sequence of Homo sapiens Lymphocyte antigen 96 (Ly96),
protein
UniProtKB/Swiss-Prot: LY96_HUMAN, Q9Y6Y9
MLPFLFFSTLFSSIFTEAQKAYWVCNSSDASISYTYCDKMQYPISINVNPCIELKRSKGLLHIFYIPRRDLKQLYFNLYITVNT
MNLPKRKEVICRGSDDDYSFCRALKGETVNTTISFSFKGIKFSKGKYKCVVEAISGSPEEMLFCLEFVILHQPNSN
SEQ ID No. 9: Nucleotide sequence encoding Homo sapiens Annexin A2 (ANXA2), mRNA
NCBI Reference Sequence: NM_001002858.2. The coding region ranges from nucleotide 74
to nucleotide 1147 (highlighted in bold). It is understood that the mRNA
corresponds to the sequence below (i.e. is identical to that sequence) with the
exception that the “t” (thymidine) residue is replaced by a “uracil” (u) residue.
ORIGIN
            1 gctcagcatt tggggacgct ctcagctctc ggcgcacggc ccaggtaagc ggggcgcgcc
          61 ctgcccgccc gcgatgggcc gccagctagc ggggtgtgga gacgctggga agaaggcttc
        121 cttcaaaatg tctactgttc acgaaatcct gtgcaagctc agcttggagg gtgatcactc
        181 tacaccccca agtgcatatg ggtctgtcaa agcctatact aactttgatg ctgagcggga
        241 tgctttgaac attgaaacag ccatcaagac caaaggtgtg gatgaggtca ccattgtcaa
        301 cattttgacc aaccgcagca atgcacagag acaggatatt gccttcgcct accagagaag
        361 gaccaaaaag gaacttgcat cagcactgaa gtcagcctta tctggccacc tggagacggt
        421 gattttgggc ctattgaaga cacctgctca gtatgacgct tctgagctaa aagcttccat
        481 gaaggggctg ggaaccgacg aggactctct cattgagatc atctgctcca gaaccaacca
        541 ggagctgcag gaaattaaca gagtctacaa ggaaatgtac aagactgatc tggagaagga
        601 cattatttcg gacacatctg gtgacttccg caagctgatg gttgccctgg caaagggtag
        661 aagagcagag gatggctctg tcattgatta tgaactgatt gaccaagatg ctcgggatct
        721 ctatgacgct ggagtgaaga ggaaaggaac tgatgttccc aagtggatca gcatcatgac
        781 cgagcggagc gtgccccacc tccagaaagt atttgatagg tacaagagtt acagccctta
        841 tgacatgttg gaaagcatca ggaaagaggt taaaggagac ctggaaaatg ctttcctgaa
        901 cctggttcag tgcattcaga acaagcccct gtattttgct gatcggctgt atgactccat
        961 gaagggcaag gggacgcgag ataaggtcct gatcagaatc atggtctccc gcagtgaagt
      1021 ggacatgttg aaaattaggt ctgaattcaa gagaaagtac ggcaagtccc tgtactatta
      1081 tatccagcaa gacactaagg gcgactacca gaaagcgctg ctgtacctgt gtggtggaga
      1141 tgactgaagc ccgacacggc ctgagcgtcc agaaatggtg ctcaccatgc ttccagctaa
      1201 caggtctaga aaaccagctt gcgaataaca gtccccgtgg ccatccctgt gagggtgacg
      1261 ttagcattac ccccaacctc attttagttg cctaagcatt gcctggcctt cctgtctagt
      1321 ctctcctgta agccaaagaa atgaacattc caaggagttg gaagtgaagt ctatgatgtg
      1381 aaacactttg cctcctgtgt actgtgtcat aaacagatga ataaactgaa tttgtacttt
      1441 agaaacacgt actttgtggc cctgctttca actgaattgt ttgaaaatta aacgtgcttg
      1501 gggttcagct ggtgaggctg tccctgtagg aagaaagctc tgggactgag ctgtacagta
      1561 tggttgcccc tatccaagtg tcgctattta agttaaattt aaatgaaata aaataaaata
      1621 aaatcaaaaa aa
//
SEQ ID No. 10: Amino acid sequence of Homo sapiens Annexin A2 (ANXA2), protein
UniProtKB/Swiss-Prot: ANAX2_HUMAN, P07355
MSTVHEILCKLSLEGDHSTPPSAYGSVKAYTNFDAERDALNIETAIKTKGVDEVTIVNILTNRSNAQRQDIAFAYQRRTKKELA
SALKSALSGHLETVILGLLKTPAQYDASELKASMKGLGTDEDSLIEIICSRTNQELQEINRVYKEMYKTDLEKDIISDTSGDFR
KLMVALAKGRRAEDGSVIDYELIDQDARDLYDAGVKRKGTDVPKWISIMTERSVPHLQKVFDRYKSYSPYDMLESIRKEVKGDL
ENAFLNLCQCIQNKPLYFADRLYDSMKGKGTRDKVLIRIMVSRSEVDMLKIRSEFKRKYGKSLYYYIQQDTKGDYQKALLYLGG
DD
SEQ ID No. 11: Nucleotide sequence encoding Homo sapiens CD86 Molecule (CD86), mRNA
NCBI Reference Sequence: NM_001206924.1. The coding region ranges from nucleotide
129 to nucleotide 782 (highlighted in bold). It is understood that the mRNA
corresponds to the sequence below (i.e. is identical to that sequence) with the
exception that the “t” (thymidine) residue is replaced by a “uracil” (u) residue.
ORIGIN
            1 agtcattgcc gaggaaggct tgcacagggt gaaagctttg cttctctgct gctgtaacag
          61 ggactagcac agacacacgg atgagtgggg tcatttccag atattaggtc acagcagaag
        121 cagccaaaat ggatccccag tgcactatgg gactgagtaa cattctcttt gtgatggctt
        181 tcctgctctc tgctaacttc agtcaacctg aaatagtacc aatttctaat ataacagaaa
        241 atgtgtacat aaatttgacc tgctcatcta tacacggtta cccagaacct aagaagatga
        301 gtgttttgct aagaaccaag aattcaacta tcgagtatga tggtattatg cagaaatctc
        361 aagataatgt cacagaactg tacgacgttt ccatcagctt gtctgtttca ttccctgatg
        421 ttacgagcaa tatgaccatc ttctgtattc tggaaactga caagacgcgg cttttatctt
        481 cacctttctc tatagagctt gaggaccctc agcctccccc agaccacatt ccttggatta
        541 cagctgtact tccaacagtt attatatgtg tgatggtttt ctgtctaatt ctatggaaat
        601 ggaagaagaa gaagcggcct cgcaactctt ataaatgtgg aaccaacaca atggagaggg
        661 aagagagtga acagaccaag aaaagagaaa aaatccatat acctgaaaga tctgatgaag
        721 cccagcgtgt ttttaaaagt tcgaagacat cttcatgcga caaaagtgat acatgttttt
        781 aattaaagag taaagcccat acaagtattc attttttcta ccctttcctt tgtaagttcc
        841 tgggcaacct ttttgatttc ttccagaagg caaaaagaca ttaccatgag taataagggg
        901 gctccaggac tccctctaag tggaatagcc tccctgtaac tccagctctg ctccgtatgc
        961 caagaggaga ctttaattct cttactgctt cttttcactt cagagcacac ttatgggcca
      1021 agcccagctt aatggctcat gacctggaaa taaaatttag gaccaatacc tcctccagat
      1081 cagattcttc tcttaatttc atagattgtg tttttttttt aaatagacct ctcaatttct
      1141 ggaaaactgc cttttatctg cccagaattc taagctggtg ccccactgaa ttttgtgtac
      1201 ctgtgactaa acaactacct cctcagtctg ggtgggactt atgtatttat gaccttatag
      1261 tgttaatatc ttgaaacata gagatctatg tactgtaata gtgtgattac tatgctctag
      1321 agaaaagtct acccctgcta aggagttctc atccctctgt cagggtcagt aaggaaaacg
      1381 gtggcctagg gtacaggcaa caatgagcag accaacctaa atttggggaa attaggagag
      1441 gcagagatag aacctggagc cacttctatc tgggctgttg ctaatattga ggaggcttgc
      1501 cccacccaac aagccatagt ggagagaact gaataaacag gaaaatgcca gagcttgtga
      1561 accctgtttc tcttgaagaa ctgactagtg agatggcctg gggaagctgt gaaagaacca
      1621 aaagagatca caatactcaa aagagagaga gagagaaaaa agagagatct tgatccacag
      1681 aaatacatga aatgtctggt ctgtccaccc catcaacaag tcttgaaaca agcaacagat
      1741 ggatagtctg tccaaatgga cataagacag acagcagttt ccctggtggt cagggagggg
      1801 ttttggtgat acccaagtta ttgggatgtc atcttcctgg aagcagagct ggggagggag
      1861 agccatcacc ttgataatgg gatgaatgga aggaggctta ggactttcca ctcctggctg
      1921 agagaggaag agctgcaacg gaattaggaa gaccaagaca cagatcaccc ggggcttact
      1981 tagcctacag atgtcctacg ggaacgtggg ctggcccagc atagggctag caaatttgag
      2041 ttggatgatt gtttttgctc aaggcaacca gaggaaactt gcatacagag acagatatac
      2101 tgggagaaat gactttgaaa acctggctct aaggtgggat cactaaggga tggggcagtc
      2161 tctgcccaaa cataaagaga actctgggga gcctgagcca caaaaatgtt cctttatttt
      2221 atgtaaaccc tcaagggtta tagactgcca tgctagacaa gcttgtccat gtaatattcc
      2281 catgttttta ccctgcccct gccttgatta gactcctagc acctggctag tttctaacat
      2341 gttttgtgca gcacagtttt taataaatgc ttgttacatt catttaaaaa aaaaaaaaa
//
SEQ ID No. 12: Amino acid sequence of Homo sapiens CD86 Molecule (CD86), protein
UniProtKB/Swiss-Prot: CD86_HUMAN, P42081
MDPQCTMGLSNILFVMAFLLSGAAPLKIQAYFNETADLPCQFANSQNQSLSELVVFWQDQENLVLNEVYLGKEKFDSVHSKYMG
RTSFDSDWTLRLHNLQIKDKGLYQCIIHHKKPTGMIRIHQMNSELSVLANFSQPEIVPISNITENVYINLTCSSIHGYPEPKKM
SVLLRTKNSTIEYDGVMQKSQDNVTELYDVSISLSVSFPDVTSNMTIFCILETDKTRLLSSPFSIELEDPQPPPDHIPWITAVL
PTVIICVMVFCLILWKWKKKKRPRNSYKCGTNTMEREESEQTKKREKIHIPERSDEAQRVFKSSTSSCDKSDTCF
SEQ ID No. 13: Nucleotide sequence encoding Homo sapiens G protein-coupled
receptor 65 (GPR65), mRNA
NCBI Reference Sequence: NM_003608.3. The coding region ranges from nucleotide
559 to nucleotide 1572 (highlighted in bold). It is understood that the mRNA
corresponds to the sequence below (i.e. is identical to that sequence) with the
exception that the “t” (thymidine) residue is replaced by a “uracil” (u) residue.
ORIGIN
            1 cctgtcccct cagcagtgtt ggtttctctt cttgacttga tgcaggcaca gatttatcaa
          61 gctcctcagt caacaaacac atcaccggaa gaaatatgga aggaaaggaa ttttaaaagg
        121 aaataccaat ctctgtgcaa acaaagcctt gtatattcat gtttgcacca atctactgtg
        181 agatttatga agaaaaacaa attgcggaca actctctatg tacacttaca aatgcctcag
        241 ttgatgcttg tgggctgttt gtcagcgttc tgtgataatg aacacatgga cttctgttta
        301 ttaaattcag ttgacccctt tagccaattg ccaggagcct ggatttttac ttccaactgc
        361 tgatatctgt gtaaaaattg atctacatcc accctttaaa agcattgatg aattaattag
        421 aactttagac aacaaagaaa aattgaaaaa gaattctcag taaaagcgaa ttcgatgttc
        481 aaaacaaact acaaagagac aagacttctc tgtttacttt ctaagaacta atataattgc
        541 taccttaaaa aggaaaaaat gaacagcaca tgtattgaag aacagcatga cctggatcac
        601 tatttgtttc ccattgttta catctttgtg attatagtca gcattccagc caatattgga
        661 tctctgtgtg tgtctttcct gcaagcaaag aaggaaagtg aactaggaat ttacctcttc
        721 agtttgtcac tatcagattt actctatgca ttaactctcc ctttatggat tgattatacc
        781 tggaataaag acaactggac tttctctcct gccttgtgca aagggagtgc ttttctcatg
        841 tacatgaatt tttacagcag cacagcattc ctcacctgca ttgccgttga tcggtatttg
        901 gctgttgtct accctttgaa gttttttttc ctaaggacaa gaagatttgc actcatggtc
        961 agcctgtcca tctggatatt ggaaaccatc ttcaatgctg tcatgttgtg ggaagatgaa
      1021 acagttgttg aatattgcga tgccgaaaag tctaatttta ctttatgcta tgacaaatac
      1081 cctttagaga aatggcaaat caacctcaac ttgttcagga cgtgtacagg ctatgcaata
      1141 cctttggtca ccatcctgat ctgcaaccgg aaagtctacc aagctgtgcg gcacaataaa
      1201 gccacggaaa acaaggaaaa gaagagaatc ataaaactac ttgtcagcat cacagttact
      1261 tttgtcttat gctttactcc ctttcatgtg atgttgctga ttcgctgcat tttagagcat
      1321 gctgtgaact tcgaagacca cagcaattct gggaagcgaa cttacacaat gtatagaatc
      1381 acggttgcat taacaagttt aaattgtgtt gctgatccaa ttctgtactg ttttgtaacc
      1441 gaaacaggaa gatatgatat gtggaatata ttaaaattct gcactgggag gtgtaataca
      1501 tcacaaagac aaagaaaacg catactttct gtgtctacaa aagatactat ggaattagag
      1561 gtccttgagt agaaccaagg atgttttgaa gggaagggaa gtttaagtta tgcattatta
      1621 tatcatcaag attacatttt gaaaaggaaa tctagcatgt gaggggacta agtgttctca
      1681 gagtgatgtt ttaatccagt ccaataaaaa tatcttaaaa ctgcattgta cagctccctc
      1741 cctgcgtttt attaaatgat gtatattaaa caaagatcaa tattttctta atgactcagg
      1801 gtctttattg ttaatgccaa ttgtttttgt atctgtgcta taatccctta gagtcagtaa
      1861 agtatgtagg ggactgtttc ttcctttgtg tctgggttta tgatttttct cactctttct
      1921 ttggactcca gggtgtcagc catcaggtct cctaattttg tgtaccggtc tccaacaacc
      1981 ccagctactg aatactgctt ctaatctcct cattcattaa caaatcttta tttttttatc
      2041 ttgtataaaa taactgcttt attgacacaa aatttacata acttaaaatt caactttgta
      2101 ttgtgtacaa ttcagtgatt ttttgtatat tcacagagct gtgcaaccat caccacactc
      2161 aaaaaatttt catcacccac caaagaaatc ttatactctt agcagtcgct ccctgctctc
      2221 ccgtccatgc cagttattaa tttactttct gtctctaagg attttcatta ctctgaacat
      2281 ttcatataaa tagaattata caatatgtgg cctactgtga cgtatttcac ttagtataat
      2341 ggtttcaagt tttatccatg tgtagaatgt atcagcactt catttctttt tatggcctga
      2401 tagtattctg ttgcatggtt atactccatt ttgtttatct aatcacttgg cttcattaac
      2461 aaatatttat tgaatccatt ccataaacta ggttttgagt taagtactgg ggctatgaaa
      2521 gaaatggtct catgaagcct cacgaagttt acattagttc aaaagcctag tcaccgagct
      2581 tgaaagattt ctatataaag gaaaaggaaa taggctctga gttttatttt gatctctttt
      2641 taatttataa ctgggtataa catagctgaa attaccagaa gtttaatgca tagacaaata
      2701 aatagttcta ttatatcttt ctttttggac ttagaatgtt agaatatttt gagagttctt
      2761 tttttttttt tttttgagtc agagtcttgc tctgtaatcc aggctagagt gtagtggtgc
      2821 gatctccact cactgcagcc tccacctccc aggttcaagc gattctcctg cctcagcctc
      2881 ccaagtagct gggattacag gcacccacca ccatgcccag ctaatttttg tatttttagt
      2941 agagacgggg tttcaccatg ttgcacaggc tggtctcaat cgaactcctg acctcaagtg
      3001 atcatcccac ctaggtctcc caaagtgctg agatgacagg cgtgagccac catgcctggc
      3061 aaagagagtc ttgatacaac atattctttt gaatcctcat tgtgtaaatt gcctcgttgt
      3121 aaatagacac tcagtaaaca ttttcctcac caaaatattt ttaaggattt ttctaccctt
      3181 ctccttttct ctttgctttc cttttcttgc ctgttctttc cactcccccc aaaatgatca
      3241 gatagcaaat gtcttgataa catgaggtgc cctcacatta aaaaacaaaa tattgagccg
      3301 ggcgcggtgg ctcatgcctg taatcccagc actttgggag gctgaggtgg gcagatcgcc
      3361 ttaggtcagg agttggagac caggctgacc aatatgatga aactctgtct ctactaaaaa
      3421 ttcaaaaatg tgccagacct ggcctggtgg catgtgcctg taatcccagc tacttgggag
      3481 gctgagtcat aagcctgcaa tgggaaaatg gatcgaatct ggggtgaggg ggaagtgatg
      3541 tgggggttat ggtacctctt ttctcttcca aagatgctgt tcttactgca tcacttgtgg
      3601 ctggccagga aaagccatgc aggagttttg tttgtggcca ctaggtgacg atcgtgttct
      3661 gtacgggacc tcttattaat agttcaccac tagccgccac tccagaagag cggaggaacc
      3721 caggataata ttttgtcaac caagaaacaa gaagtccctc ccaggaactg gaaatgaatg
      3781 gggaaaatgc tgaaatctca tttgcactat tcatttctct tctctctgga aagctcggca
      3841 atcatcaggt catttcattt ggcttaaatt ccatgtgtct ttccaaactt ttaaaagctg
      3901 gtgaaaattg ttccacccat atgtaaaaga acataggtta agttgtctaa ttcttgcagg
      3961 aatgtggata tagcattaaa aatatgtctt tgtatactta tcttacccat gtaagaaaag
      4021 agtggccaac tttcatataa atagaaagag aacatttaag ctatatgcag tttgcatttt
      4081 tgtctactat tatgaaatta ttatctatga aattcaagct gtaactcaac atatgtataa
      4141 ttttaatttc taatttattg ttagatctca gcacttaaaa aattacatct tgtatttgaa
      4201 ttgttaaatc tgttccctgc aaagaacagt aatacaatca tgttctaatt tactagcatt
      4261 tgcatatttt agaaatataa tggcctgtaa tttacttttc ttttgcctat aattttctga
      4321 agctctttat gatgcaccgg tgcattttta tttaaaaaat agattgtgac tcctcaaata
      4381 atgttacaat tcgatgttca aaaagcaatc caggtacata gccataaagg gatgagctag
      4441 agaggtctcc atattatcat tcaatgtgag aataaaaatt ctatatttta ttctagaata
      4501 aaattataaa tttctttatc ta //
SEQ ID No. 14: Amino acid sequence of Homo sapiens G protein-coupled receptor 65
(GPR65), protein
UniProtKB/Swiss-Prot: PSYR_HUMAN, Q8IYL9
MNSTCIEEQHDLDHYLFPIVYIFVIIVSIPANIGSLCVSFLQAKKESELGIYLFSLSLSDLLYALTLPLWIDYTWNKDNWTFSP
ALCKGSAFLMYMNFYSSTAFLTCIAVDRYLAVVYPLKFFFLRTRRFALMVSLSIWILETIFNAVMLWEDETVVEYCDAEKSNFT
LCYDKYPLEKWQINLNLFRTCTGYAIPLVTILICNRKVYQAVRHNKATENKEKKRIIKLLVSITVTFVLCFTPFHVMLLIRCIL
EHAVNFEDHSNSGKRTYTMYRITVALTSLNCVADPILYCFVTETGRYDMWNILKFCTGRCNTSQRQRKRILSVSTKDTMELEVL
E
SEQ ID No. 15: Nucleotide sequence encoding Homo sapiens Peptidase inhibitor 16
(CRISP9), mRNA
NCBI Reference Sequence: NM_153370.2. The coding region ranges from nucleotide
329 to nucleotide 1720 (highlighted in bold). It is understood that the mRNA
corresponds to the sequence below (i.e. is identical to that sequence) with the
exception that the “t” (thymidine) residue is replaced by a “uracil” (u) residue.
ORIGIN
            1 cctgggtgca accagtcaca gctctgcaga ggttactgtg attttgcccc tgaaggatct
          61 gtccacaact taggaactca cacagctttt ggcctgagcc cccgttacca agagaaagga
        121 ggtttttgcc aaggactcca aggggagtgc acttgatgct ggtcgggacc caaagcgccc
        181 agccctccct gagacattgt gtgagtcggg ctgggcctca aacacggccc ccactgcccc
        241 accccagcca gggtggtgct tgtgtgggaa ggactttaaa tccagctgcc agacccctgg
        301 acgggagaag gagagacggc tggccaccat gcacggctcc tgcagtttcc tgatgcttct
        361 gctgccgcta ctgctactgc tggtggccac cacaggcccc gttggagccc tcacagatga
        421 ggagaaacgt ttgatggtgg agctgcacaa cctctaccgg gcccaggtat ccccgacggc
        481 ctcagacatg ctgcacatga gatgggacga ggagctggcc gccttcgcca aggcctacgc
        541 acggcagtgc gtgtggggcc acaacaagga gcgcgggcgc cgcggcgaga atctgttcgc
        601 catcacagac gagggcatgg acgtgccgct ggccatggag gagtggcacc acgagcgtga
        661 gcactacaac ctcagcgccg ccacctgcag cccaggccag atgtgcggcc actacacgca
        721 ggtggtatgg gccaagacag agaggatcgg ctgtggttcc cacttctgtg agaagctcca
        781 gggtgttgag gagaccaaca tcgaattact ggtgtgcaac tatgagcctc cggggaacgt
        841 gaaggggaaa cggccctacc aggaggggac tccgtgctcc caatgtccct ctggctacca
        901 ctgcaagaac tccctctgtg aacccatcgg aagcccggaa gatgctcagg atttgcctta
        961 cctggtaact gaggccccat ccttccgggc gactgaagca tcagactcta ggaaaatggg
      1021 tactccttct tccctagcaa cggggattcc ggctttcttg gtaacagagg tctcaggctc
      1081 cctggcaacc aaggctctgc ctgctgtgga aacccaggcc ccaacttcct tagcaacgaa
      1141 agacccgccc tccatggcaa cagaggctcc accttgcgta acaactgagg tcccttccat
      1201 tttggcagct cacagcctgc cctccttgga tgaggagcca gttaccttcc ccaaatcgac
      1261 ccatgttcct atcccaaaat cagcagacaa agtgacagac aaaacaaaag tgccctctag
      1321 gagcccagag aactctctgg accccaagat gtccctgaca ggggcaaggg aactcctacc
      1381 ccatgcccag gaggaggctg aggctgaggc tgagttgcct ccttccagtg aggtcttggc
      1441 ctcagttttt ccagcccagg acaagccagg tgagctgcag gccacactgg accacacggg
      1501 gcacacctcc tccaagtccc tgcccaattt ccccaatacc tctgccaccg ctaatgccac
      1561 gggtgggcgt gccctggctc tgcagtcgtc cttgccaggt gcagagggcc ctgacaagcc
      1621 tagcgtcgtg tcagggctga actcgggccc tggtcatgtg tggggccctc tcctgggact
      1681 actgctcctg cctcctctgg tgttggctgg aatcttctga aggggatacc actcaaaggg
      1741 tgaagaggtc agctgtcctc ctgtcatctt ccccaccctg tccccagccc ctaaacaaga
      1801 tacttcttgg ttaaggccct ccggaaggga aaggctacgg ggcatgtgcc tcatcacacc
      1861 atccatcctg gaggcacaag gcctggctgg ctgcgagctc aggaggccgc ctgaggactg
      1921 cacaccgggc ccacacctct cctgcccctc cctcctgagt cctgggggtg ggaggatttg
      1981 agggagctca ctgcctacct ggcctggggc tgtctgccca cacagcatgt gcgctctccc
      2041 tgagtgcctg tgtagctggg gatggggatt cctaggggca gatgaaggac aagccccact
      2101 ggagtggggt tctttgagtg ggggaggcag ggacgaggga aggaaagtaa ctcctgactc
      2161 tccaataaaa acctgtccaa cctgtggcaa aaaaaaaaaa aaaaa
//
SEQ ID No. 16: Amino acid sequence of Homo sapiens Peptidase inhibitor 16 (CRISP9),
protein
UniProtKB/Swiss-Prot: PI16_HUMAN, Q6UXB8
MHGSCSFLMLLLPLLLLLVATTGPVGALTDEEKRLMVELHNLYRAQVSPTASDMLHMRWDEELAAFAKAYARQCVWGHNKERGR
RGENLFAITDEGMDVPLAMEEWHHEREHYNLSAATCSPGQMCGHYTQVVWAKTERIGCGSHFCEKLQGVEETNIELLVCNYEPP
GNVKGKRPYQEGTPCSQCPSGYHCKNSLCEPIGSPEDAQDLPYLVTEAPSFRATEASDSRKMGTPSSLATGIPAFLVTEVSGSL
ATKALPAVETQAPTSLATKDPPSMATEAPPCVTTEVPSILAAHSLPSLDEEPVTFPKSTHVPIPKSADKVTDKTKVPSRSPENS
LDPKMSLTGARELLPHAQEEAEAEAELPPSSEVLASVFPAQDKPGELQATLDHTGHTSSKSLPNFPNTSATANATGGRALAQSS
LPGAEGPDKPSVVSGLNSGPGHVWGPLLGLLLLPPLVLAGIF
SEQ ID No. 17: Nucleotide sequence encoding Homo sapiens Lysozyme (LYZ), mRNA
NCBI Reference Sequence: NM_000239.2. The coding region ranges from nucleotide
56 to nucleotide 502 (highlighted in bold). It is understood that the mRNA
corresponds to the sequence below (i.e. is identical to that sequence) with the
exception that the “t” (thymidine) residue is replaced by a “uracil” (u) residue.
ORIGIN
            1 aaatactggg gccagctcac cctggtcagc ctagcactct gacctagcag tcaacatgaa
          61 ggctctcatt gttctggggc ttgtcctcct ttctgttacg gtccagggca aggtctttga
        121 aaggtgtgag ttggccagaa ctctgaaaag attgggaatg gatggctaca ggggaatcag
        181 cctagcaaac tggatgtgtt tggccaaatg ggagagtggt tacaacacac gagctacaaa
        241 ctacaatgct ggagacagaa gcactgatta tgggatattt cagatcaata gccgctactg
        301 gtgtaatgat ggcaaaaccc caggagcagt taatgcctgt catttatcct gcagtgcttt
        361 gctgcaagat aacatcgctg atgctgtagc ttgtgcaaag agggttgtcc gtgatccaca
        421 aggcattaga gcatgggtgg catggagaaa tcgttgtcaa aacagagatg tccgtcagta
        481 tgttcaaggt tgtggagtgt aactccagaa ttttccttct tcagctcatt ttgtctctct
        541 cacattaagg gagtaggaat taagtgaaag gtcacactac cattatttcc ccttcaaaca
        601 aataatattt ttacagaagc aggagcaaaa tatggccttt cttctaagag atataatgtt
        661 cactaatgtg gttattttac attaagccta caacattttt cagtttgcaa atagaactaa
        721 tactggtgaa aatttaccta aaaccttggt tatcaaatac atctccagta cattccgttc
        781 tttttttttt tgagacagtc tcgctctgtc gcccaggctg gagtgcagtg gcgcaatctc
        841 ggctcactgc aacctccacc tcccgggttc acgccattct cctgcctcag cctcccgagt
        901 agctgggatt acgggcgccc gccaccacgc ccggctaatt ttttgtattt ttagtagaga
        961 cagggtttca ccgtgttagc caggatggtc tcgatctcct gaccttgtga tccacccacc
      1021 tcggcctccc aaagtgctgg gattacaggc gtgagccact gcgcccggcc acattcagtt
      1081 cttatcaaag aaataaccca gacttaatct tgaatgatac gattatgccc aatattaagt
      1141 aaaaaatata agaaaaggtt atcttaaata gatcttaggc aaaataccag ctgatgaagg
      1201 catctgatgc cttcatctgt tcagtcatct ccaaaaacag taaaaataac cactttttgt
      1261 tgggcaatat gaaattttta aaggagtaga ataccaaatg atagaaacag actgcctgaa
      1321 ttgagaattt tgatttctta aagtgtgttt ctttctaaat tgctgttcct taatttgatt
      1381 aatttaattc atgtattatg attaaatctg aggcagatga gcttacaagt attgaaataa
      1441 ttactaatta atcacaaatg tgaagttatg catgatgtaa aaaatacaaa cattctaatt
      1501 aaaggctttg caacac
//
SEQ ID No. 18: Amino acid sequence of Homo sapiens Lysozyme (LYZ), protein
UniProtKB/Swiss-Prot: LYSC_HUMAN, P61626
MKALIVLGLVLLSVTVQGKVFERCELARTLKRLGMDGYRGISLANWMCLAKWESGYNTRATNYNAGDRSTDYGIFQINSRYWCN
DGKTPGAVNACHLSCSALLQDNIADAVACAKRVVRDPQGRIAWVAWRNRCQNRDVRQYVQGCGV
SEQ ID No. 19: Nucleotide sequence encoding Homo sapiens Vimentin (VIM), mRNA
NCBI Reference Sequence: NM_003380.3. The coding region ranges from nucleotide
414 to nucleotide 1814 (highlighted in bold). It is understood that the mRNA
corresponds to the sequence below (i.e. is identical to that sequence) with the
exception that the “t” (thymidine) residue is replaced by a “uracil” (u) residue.
ORIGIN
            1 gcctctccaa aggctgcaga agtttcttgc taacaaaaag tccgcacatt cgagcaaaga
          61 caggctttag cgagttatta aaaacttagg ggcgctcttg tcccccacag ggcccgaccg
        121 cacacagcaa ggcgatggcc cagctgtaag ttggtagcac tgagaactag cagcgcgcgc
        181 ggagcccgct gagacttgaa tcaatctggt ctaacggttt cccctaaacc gctaggagcc
        241 ctcaatcggc gggacagcag ggcgcgtcct ctgccactct cgctccgagg tccccgcgcc
        301 agagacgcag ccgcgctccc accacccaca cccaccgcgc cctcgttcgc ctcttctccg
        361 ggagccagtc cgcgccaccg ccgccgccca ggccatcgcc accctccgca gccatgtcca
        421 ccaggtccgt gtcctcgtcc tcctaccgca ggatgttcgg cggcccgggc accgcgagcc
        481 ggccgagctc cagccggagc tacgtgacta cgtccacccg cacctacagc ctgggcagcg
        541 cgctgcgccc cagcaccagc cgcagcctct acgcctcgtc cccgggcggc gtgtatgcca
        601 cgcgctcctc tgccgtgcgc ctgcggagca gcgtgcccgg ggtgcggctc ctgcaggact
        661 cggtggactt ctcgctggcc gacgccatca acaccgagtt caagaacacc cgcaccaacg
        721 agaaggtgga gctgcaggag ctgaatgacc gcttcgccaa ctacatcgac aaggtgcgct
        781 tcctggagca gcagaataag atcctgctgg ccgagctcga gcagctcaag ggccaaggca
        841 agtcgcgcct gggggacctc tacgaggagg agatgcggga gctgcgccgg caggtggacc
        901 agctaaccaa cgacaaagcc cgcgtcgagg tggagcgcga caacctggcc gaggacatca
        961 tgcgcctccg ggagaaattg caggaggaga tgcttcagag agaggaagcc gaaaacaccc
      1021 tgcaatcttt cagacaggat gttgacaatg cgtctctggc acgtcttgac cttgaacgca
      1081 aagtggaatc tttgcaagaa gagattgcct ttttgaagaa actccacgaa gaggaaatcc
      1141 aggagctgca ggctcagatt caggaacagc atgtccaaat cgatgtggat gtttccaagc
      1201 ctgacctcac ggctgccctg cgtgacgtac gtcagcaata tgaaagtgtg gctgccaaga
      1261 acctgcagga ggcagaagaa tggtacaaat ccaagtttgc tgacctctct gaggctgcca
      1321 accggaacaa tgacgccctg cgccaggcaa agcaggagtc cactgagtac cggagacagg
      1381 tgcagtccct cacctgtgaa gtggatgccc ttaaaggaac caatgagtcc ctggaacgcc
      1441 agatgcgtga aatggaagag aactttgccg ttgaagctgc taactaccaa gacactattg
      1501 gccgcctgca ggatgagatt cagaatatga aggaggaaat ggctcgtcac cttcgtgaat
      1561 accaagacct gctcaatgtt aagatggccc ttgacattga gattgccacc tacaggaagc
      1621 tgctggaagg cgaggagagc aggatttctc tgcctcttcc aaacttttcc tccctgaacc
      1681 tgagggaaac taatctggat tcactccctc tggttgatac ccactcaaaa aggacacttc
      1741 tgattaagac ggttgaaact agagatggac aggttatcaa cgaaacttct cagcatcacg
      1801 atgaccttga ataaaaattg cacacactca gtgcagcaat atattaccag caagaataaa
      1861 aaagaaatcc atatcttaaa gaaacagctt tcaagtgcct ttctgcagtt tttcaggagc
      1921 gcaagataga tttggaatag gaataagctc tagttcttaa caaccgacac tcctacaaga
      1981 tttagaaaaa agtttacaac ataatctagt ttacagaaaa atcttgtgct agaatacttt
      2041 ttaaaaggta ttttgaatac cattaaaact gctttttttt ttccagcaag tatccaacca
      2101 acttggttct gcttcaataa atctttggaa aaactcaaaa aaaaaaaaaa a
//
SEQ ID No. 20: Amino acid sequence of Homo sapiens Vimentin (VIM), protein
UniProtKB/Swiss-Prot: VIME_HUMAN, P08670
MSTRSVSSSSYRRMFGGPGTASRPSSSRSYVTTSTRTYSLGSALRPSTSRSLYASSPGGVYATRSSAVRLRSSVPGVRLLQDSV
DFSLADAINTEFKNTRTNEKVELQELNDRFANYIDKVRFLEQQNKILLAELEQLKGQGKSRLGDLYEEEMRELRRQVDQLTNDK
ARVEVERDNLAEDIMRLREKLQEEMLQREEAENTLQSFRQDVDNASLARLDLERKVESLQEEIAFLKKLHEEEIQELQAQIQEQ
HVQIDVDVSKPDLTAALRDVRQQYESVAAKNLQEAEEWYKSKFADLSEAANRNNDALRQAKQESTEYRRQVQSLTCEVDALKGT
NESLERQMREMEENFAVEAANYQDTIGRLQDEIQNMKEEMARHLREYQDLLNVKMALDIEIATYRKLLEGEESRISLPLPNFSS
LNLRETNLDSLPLVDTHSKRTLLIKTVETRDGQVINETSQHHDDLE
SEQ ID No. 21: Nucleotide sequence encoding Homo sapiens Calmodulin regulated
spectrin associated protein family member 2 (CAMSAP2), mRNA
NCBI Reference Sequence: NM_203459.2. The coding region ranges from nucleotide
271 to nucleotide 4707 (highlighted in bold). It is understood that the mRNA
corresponds to the sequence below (i.e. is identical to that sequence) with the
exception that the “t” (thymidine) residue is replaced by a “uracil” (u) residue.
ORIGIN
            1 acggggcgga cctcgcgcgg acggacggac ggagacggcg ccgccacatt cctatgcccg
          61 ggagcggcgg cggcggcggc ggcggctccc gcgggaggcg gcaggcgcgc ggcgcggaca
        121 gctgagcttc tcctccgtcg gcgcccgggc ggacatcgcc cgggccccga tggtttgagc
        181 ttgcttctcc ctccctcccg acccccgtgg tggcgaggcc acgccatgtg aaggttaggg
        241 ccgggacatc ccgaggagcc gcggtgaaag atgggggatg ctgcagaccc cagggagatg
        301 agaaagacgt tcattgttcc agccatcaag ccttttgacc actatgattt ctccagggcc
        361 aaaatcgcct gcaatctggc ctggctggtg gccaaagcct ttgggacaga aaatgtgcca
        421 gaggaacttc aagaaccatt ttacacagat cagtatgacc aggaacacat caaaccacct
        481 gttgttaatt tgcttctatc ggctgaacta tactgtcgtg ctgggagtct cattctcaag
        541 agtgatgctg caaaacccct tttgggccat gatgctgtaa tccaggcttt agcacagaaa
        601 ggtctttatg tcactgacca ggaaaaattg gtaactgaac gagatctcca caagaaaccc
        661 atacagatga gtgcacattt ggccatgatc gataccctca tgatggctta tactgtagaa
        721 atggtcagta tagaaaaagt aattgcgtgt gctcagcagt attcagcttt ttttcaagcc
        781 acagatctgc cctatgatat tgaggacgct gtcatgtact ggataaataa ggtaaatgaa
        841 catttgaaag acataatgga acaagaacaa aaactgaaag aacatcacac agttgaagct
        901 ccaggaggtc aaaaggctcg ttatcggaaa gagcaaacat tgcttaagca actgccttgc
        961 attccattgg tagaaaattt gttgaaggat gggacagatg gctgtgcatt agctgccctt
      1021 attcattttt actgtcctga tgttgtcaga ttagaggata tttgtttgaa agaaactatg
      1081 tctttggctg atagcctgta taatctgcag ctgattcaag aattttgcca agaatacttg
      1141 aaccagtgtt gccatttcac tctggaagat atgctctatg ctgcttcatc cataaagagt
      1201 aattatttgg tgttcatggc ggaactgttc tggtggtttg aagtggtgaa gccgtctttt
      1261 gtacagcctc gtgttgttcg tccacaagga gctgaacctg taaaagatat gccttcaatt
      1321 cctgtcttga atgctgccaa aagaaatgtc ttagatagta gttctgactt cccttcaagt
      1381 ggggaaggag ctacatttac acagtctcat catcatttgc cttctaggta ttcacgtccc
      1441 caggctcatt cttcagcctc aggaggaatt agaaggtctt catctatgtc ttatgttgat
      1501 ggcttcatag ggacatggcc caaagagaaa agatcatcag tgcatggcgt atcatttgat
      1561 atttcttttg ataaagaaga tagtgtacag agatccactc caaaccgagg aatcactcgt
      1621 tctattagta atgaaggact tactctgaac aacagtcatg tatctaaaca cattaggaaa
      1681 aatttgtcct tcaagccaat aaatggagaa gaggaagcag agagcattga agaagaactt
      1741 aatatagatt ctcacagtga cctcaaatct tgtgtgcccc ttaacacaaa tgaactaaat
      1801 tctaatgaga atattcatta caagcttcca aatggagctt tacaaaatag aatacttctt
      1861 gacgagtttg gcaatcagat cgagacacca agcattgaag aagcattaca aataattcag
      1921 gatactgaaa aatctcctca tacacctcag ccagaccaaa ttgctaatgg cttctttctt
      1981 catagtcaag aaatgagtat cttaaattca aatatcaagt taaatcaatc tagtcctgat
      2041 aatgtaactg atacgaaagg tgccttgagt cccataactg acaatactga agtagacact
      2101 ggaattcacg ttccttcaga agatattcct gaaactatgg acgaagattc ttcgttgaga
      2161 gattatactg taagcttgga ctctgacatg gatgatgcat ctaaatttct tcaggattat
      2221 gatattcgaa ctggcaacac cagggaagct ttgagtcctt gtccaagtac tgtaagtacc
      2281 aagtctcagc caggcagcag tgcttcttct agttctggag ttaaaatgac cagctttgct
      2341 gaacaaaaat tcaggaaact gaatcatacc gatggaaaaa gtagtggaag cagttctcaa
      2401 aaaactacac cagaaggctc tgaacttaat attcctcatg tggttgcttg ggcacaaatt
      2461 ccagaagaaa cagggcttcc acagggacgg gacactaccc agctgttggc ctctgaaatg
      2521 gtgcatctta ggatgaaact agaagaaaag aggcgtgcta tagaagccca gaaaaagaaa
      2581 atggaagctg cttttaccaa acagagacag aaaatgggaa ggacagcatt ccttactgta
      2641 gtgaaaaaga aaggggatgg gatatctcct ctacgagagg aagcggcggg tgcagaagat
      2701 gagaaagtat atactgatcg agcaaaagaa aaggaatcac aaaaaactga tggacaaagg
      2761 agcaagtcac tggcagatat aaaagagagc atggagaatc ctcaagccaa atggctaaag
      2821 tctccaacta cacctattga tcctgagaag cagtggaacc tggcaagccc ctcagaagaa
      2881 actttaaatg aaggagagat tttagaatat accaaatcca ttgaaaagtt aaattcatcc
      2941 ctgcattttc tacaacaaga aatgcaacgc ttgtcacttc agcaggagat gttaatgcag
      3001 atgagagagc aacaatcttg ggtgatttca cctccacaac cctctccaca gaaacagatt
      3061 cgagatttta agccttctaa gcaggcaggc ctgtcatcag ccattgcacc attctcctca
      3121 gactcccctc gtcctactca cccatctcca cagtcttcta acaggaaaag tgcatctttt
      3181 tctgttaaaa gtcaaaggac tcctaggcca aatgagttaa aaataacacc tttgaatcga
      3241 accttgacac ctcctcggtc tgtggatagc cttcctcggt taaggaggtt ttcaccaagt
      3301 caagttccta ttcaaactag gtcatttgta tgttttgggg atgatggaga acctcagtta
      3361 aaggaatcca aacctaaaga ggaagttaaa aaggaggaat tggaatccaa agggactttg
      3421 gaacagcgtg gacataatcc agaagaaaag gaaatcaaac cttttgagtc aacagtctct
      3481 gaagtcctat cactgcctgt cacagagact gtatgtctga caccaaatga ggaccaattg
      3541 aatcaaccca cagaaccccc tcctaaaccc gttttcccac ccactgctcc aaaaaatgtt
      3601 aatctgattg aagtttccct ctcagatttg aaaccccctg aaaaggctga tgtacctgtt
      3661 gaaaaatatg atggagaaag tgataaagaa caatttgatg atgaccagaa agtatgctgt
      3721 ggattctttt ttaaggatga tcaaaaagca gaaaatgata tggcaatgaa acgggcagct
      3781 ttgttggaga aaagattaag aagggaaaag gaaactcagc tccggaaaca acagttggaa
      3841 gcagaaatgg agcataagaa ggaggaaaca aggcgtaaaa ctgaggaaga acgtcagaag
      3901 aaagaagatg agagagcacg cagagaattt attaggcaag aatatatgag gcggaaacaa
      3961 ctgaaactaa tggaagatat ggatacagta attaaacccc gtcctcaagt agtaaaacaa
      4021 aaaaaacagc gaccaaaatc tattcacaga gatcatattg aatcccccaa aacaccaata
      4081 aagggtcctc cagtctctag cctttctttg gcatcgctga acacgggtga taacgagagt
      4141 gtacattcag gcaagaggac gccaagatca gagtctgtag aaggcttctt atctccaagt
      4201 cgttgtggca gtcgaaatgg agaaaaagac tgggagaatg catcaacaac ttcttcagtg
      4261 gcttctggaa cagaatatac aggaccaaag ctctacaaag aacccagtgc aaaatccaat
      4321 aagcacataa tacaaaatgc tttagctcat tgctgtttgg ctggaaaagt aaatgaaggt
      4381 cagaagaaaa aaatactgga ggaaatggag aaatcagatg ccaacaactt cttaatcttg
      4441 ttccgggatt caggatgcca gttcagatct ttatacactt attgcccaga aactgaagaa
      4501 atcaataaac tgactgggat aggccctaaa tctatcacta aaaaaatgat tgaaggactt
      4561 tacaaatata attctgacag gaaacagttt agccacatac ccgctaaaac tttatctgcc
      4621 agtgttgatg caattaccat tcatagccat ttatggcaga ccaaaagacc agtaacaccc
      4681 aaaaaacttt tacccactaa ggcatagaag ttgggaaata cttgcttcag aacattcatg
      4741 gtaaatttgc acttcatctt tcctgcctat agaaaatctt tctaattgcc aacaagactt
      4801 ttattaatta aaactggaca ttaagctctg ttgtcatgaa caactggaat gtaaaccaca
      4861 gtattttgga gtgcagaaca ttctcaatta agtgataagt ccaaatgatg aaggaaatgt
      4921 tttaattcac aaatggagat ttgtatgtgt tatcaggttc acctgcttga tattagatac
      4981 attaaagcac tgaattttca tggatattag ttggatttat cattgaaata tggttaagat
      5041 tacaaattat gtgttttatt tgttgctttt ttttaacctt ttaatgtata ttcttgtctt
      5101 cagatggttt gctatttttc tctcctgggg gtttattcta agataccttt gtattttatt
      5161 tcatgtggag atcatgaaag taggaaatat acctttagaa gtaactcgca cctttcttat
      5221 gatgttaaga gaaacactag tgtttagttt tacagtaacc ctcatatttt aatggtgtta
      5281 cagcatttgc aaaaattatt ctgctaagta tttacaactc tatttattat tcactcaagt
      5341 attaacattc tctattaaat aagaggaggt gttgtaaaga gctgctagta ggttcgcttt
      5401 aaaccacatg agcttaacca agaatatgtt atgagaagtt gctgattaaa tcagtgctgt
      5461 ttttacacca cttctggcca actcagaata atttagattg ttcttttaac aaaaaaggct
      5521 ttttacacca cttctggcca actcagaata atttagattg ttcttttaac aaaaaaggct
      5581 gtagtctttc aatctgaaga tgtaagactt cctgaaacaa gttctcaaga agtctttaca
      5641 ttatatttat aactcatata aaaattatat ttagaatttt taaacatgta caaagggcta
      5701 cattttaatt ttaaaatagc ttcacattat tttacttata ttgggttttt cttcatttta
      5761 atccttttca agtggaatgg cttagaataa gtatacactt gaaatctcct ctacatgatc
      5821 tttgttcttt aacagtgtat accagagggt tagttgggga aaaacttcat tctcaggaaa
      5881 agacttgaat gattatgtga ccctgttata tttcagtgtt gtgacaaatg tgtaaactag
      5941 cgggggaaga cagtattgta tcataaatga gatgcgtagt ttgttttctt tcatgggaag
      6001 tagagataaa aatatataca tttctctaat tgagttgttt agagaaagaa ctaatgtctc
      6061 atatgatgta tttacttatt ttaaaaaaaa gaataggaat gagatgtccc tgagctgtac
      6121 ttttctatta ttataaggcc tttaggcatc agtgcatctg ggttatcaac attttctcaa
      6181 atgctgtcaa tattttactg taatttatgt tcttatattt atgtatattt gttaaaactg
      6241 taaaaaaatt tcacagattt ttttccaata cctgtgcaag atacatgtgt agctcaaaac
      6301 tatttgtgat ctactgtttg catgtaagag accaggatat gtaactctta tattttaagt
      6361 gtatacatat tgtgtatata acatatggat attaaaaatg gggaattgca cattttacct
      6421 tttggacagt aatttctatc acagttagaa ggaaatgata gtcaaataca cgtttagatt
      6481 aaaactagtt taaaaaatta taaatgaatc taatcaaaat gtgaatagta gtcaaaagga
      6541 taatttaata agcattttac gttactaaat ttgttcattt caatattaac taaatttccc
      6601 tcatcaaagc aatctttgtg atattacttc gctattaaat aaagaaaatt ggatgcaaga
      6661 caatggagaa actttaaaac taaacaggac caccctttat tcttaaattt gtgtgtgtcc
      6721 aacagttgaa ttgaatgtct ataaggtcta aaggtagaat gtgaatattg ccacagagtt
      6781 cattgctctc agtataagat tttactttat taatgcagaa ggaatatgga tatatttctt
      6841 taagtctgca gattttttta ttatggtgca gctttttttt aattatgttt ttaaaattat
      6901 acagttgaaa aatatgccat ttcataaagt ctgaggattt tcgtcaacct tactgaaaca
      6961 cactggtgct ttcatcatca gaggtcaaat tattatgata actattccat taagtttgcc
      7021 aaacatttgt cgtggttacc agtgcagcct gtcaaattct gctatttgac acagctttgg
      7081 aaagatttag ttcttggttt ttccgttttg tattagaatg actgttacag ttttatttgg
      7141 ctgtttaaag ccaaattcag ctatttaatt atggtttcat ggacactgtt gagcaatgta
      7201 cagtgtatgg tgtgcttacc tgtccactct agagcattgc ttacaggttt tttgtttttt
      7261 aagatgctgt gctgtaaaat actgtcatac ttgctatttc ctggtacagt gtagtttttc
      7321 ccctttcatt tgaataaaag catggcacca aatgaaaaaa aaaaaaaaa
//
SEQ ID No. 22: Amino acid sequence of Homo sapiens Calmodulin regulated spectrin
assocaited protein family member 2 (CAMSAP2), protein
UniProtKB/Swiss-Prot: CAMP2_HUMAN, Q08AD1
MGDAADPREMRKTFIVPAIKPFDHYDFSRAKIACNLAWLVAKAFGTENVPEELQEPFYTDQYDQEHIKPPVVNLLLSAELYCRA
GSLILKSDAAKPLLGHDAVIQALAQKGLYVTDQEKLVTERDLHKKPIQMSAHLAMIDTLMMAYTVEMVSIEKVIACAQQYSAFF
QATDLPYDIEDAVMYWINKVNEHLKDIMEQEQKLKEHHTVEAPGGQKSPSKWFWKLVPARYRKEQTLLKQLPCIPLVENLLKDG
TDGCALAALIHFYCPDVVRLEDICLKETMSLADSLYNLQLIQEFCQEYLNQCCHFTLEDMLYAASSIKSNYLVFMAELFWWFEV
VKPSFVQPRVVRPQGAEPVKDMPSIPVLNAAKRNVLDSSSDFPSSGEGATFTQSHHHLPSRYSRPQAHSSASGGIRRSSSMSYV
DGFIGTWPKEKRSSVHGVSFDISFDKEDSVQRSTPNRGITRSISNEGLTLNNSHVSKHIRKNLSFKPINGEEEAESIEEELNID
SHSDLKSCVPLNTNELNSNENIHYKLPNGALQNRILLDEFGNQIETPSIEEALQIIHDTEKSPHTPQPDQIANGFFLHSQEMSI
LNSNIKLNQSSPDNVTDTKGALSPITDNTEVDTGIHVPSEDIPETMDEDSSLRDYTVSLDSDMDDASKFLQDYDIRTGNTREAL
SPCPSTVSTKSQPGSSASSSSGVKMTSFAEQKFRKLNHTDGKSSGSSSQKTTPEGSELNIPHVVAWAQIPEETGLPQGRDTTQL
LASEMVHLRMKLEEKRRAIEAQKKKMEAAFTKQRQKMGRTAFLTVVKKKGDGISPLREEAAGAEDEKVYTDRAKEKESQKTDGQ
RSKSLADIKESMENPQAKWLKSPTTPIDPEKQWNLASPSEETLNEGEILEYTKSIEKLNSSLHFLQQEMQRLSLQQEMLMQMRE
QQSWVISPPQPSPQKQIRDFKPSKQAGLSSAIAPFSSDSPRPTHPSPQSSNRKSASFSVKSQRTPRPNELKITPLNRTLTPPRS
VDSLPRLRRFSPSQVPIQTRSFVCFGDDGEPQLKESKPKEEVKKEELESKGTLEQRGHNPEEKIEKPFESTVSEVLSLPVTETV
CLTPNEDQLNQPTEPPPKPVFPPTAPKNVNLIEVSLSDLKPPEKADVPVEKYDGESDKEQFDDDQKVCCGFFFKDDQKAENDMA
MKRAALLEKRLRREKETQLRKQQLEAEMEHKKEETRRKTEEERQKKEDERARREFIRQEYMRRKQLKLMEDMDTVIKPRPQVVK
QKKQRPKSIHRDHIESPKTPIKGPPVSSLSLASLNTGDNESVHSGKRTPRSESVEGFLSPSRCGSRNGEKDWENASTTSSVASG
TEYTGPKLYKEPSAKSNKHIIQNALAHCCLAGKVNEGQKKKILEEMEKSDANNFLILFRDSGCQFRSLYTYCPETEEINKLTGI
GPKSITKKMIEGLYKYNSDRKQFSHIPAKTLSASVDAITIHSHLWQTKRPVTPKKLLPTKA
SEQ ID No. 23: Nucleotide sequence encoding Homo sapiens Cathepsin G (CTSG), mRNA
NCBI Reference Sequence: NM_. The coding region ranges from nucleotide
38 to nucleotide 805 (highlighted in bold). It is understood that the mRNA
corresponds to the sequence below (i.e. is identical to that sequence) with the
exception that the “t” (thymidine) residue is replaced by a “uracil” (u) residue.
ORIGIN
          1 gcacagcagc aactgactgg gcagcctttc aggaaagatg cagccactcc tgcttctgct
        61 ggcctttctc ctacccactg gggctgaggc aggggagatc atcggaggcc gggagagcag
      121 gccccactcc cgcccctaca tggcgtatct tcagatccag agtccagcag gtcagagcag
      181 atgtggaggg ttcctggtgc gagaagactt tgtgctgaca gcagctcatt gctggggaag
      241 caatataaat gtcaccctgg gcgcccacaa tatccagaga cgggaaaaca cccagcaaca
      301 catcactgcg cgcagagcca tccgccaccc tcaatataat cagcggacca tccagaatga
      361 catcatgtta ttgcagctga gcagaagagt cagacggaat cgaaacgtga acccagtggc
      421 tctgcctaga gcccaggagg gactgagacc cgggacgctg tgcactgtgg ccggctgggg
      481 cagggtcagc atgaggaggg gaacagatac actccgagag gtgcagctga gagtgcagag
      541 ggataggcag tgcctccgca tcttcggttc ctacgacccc cgaaggcaga tttgtgtggg
      601 ggaccggcgg gaacggaagg ctgccttcaa gggggattcc ggaggccccc tgctgtgtaa
      661 caatgtggcc cacggcatcg tctcctatgg aaagtcgtca ggggttcctc cagaagtctt
      721 caccagggtc tcaagtttcc tgccctggat aaggacaaca atgagaagct tcaaactgct
      781 ggatcagatg gagacccccc tgtgactgac tcttcttctc ggggacacag gccagctcca
      841 cagtgttgcc agagccttaa taaacgtcca cagagtataa ataaccaatt cctcatttgt
      901 tcattaaacg tcattcagta ctta //
SEQ ID No. 24: Amino acid sequence of Homo sapiens Cathepsin G (CTSG), protein
UniProtKB/Swiss-Prot: CATG_HUMAN, P08311
MQPLLLLLAFLLPTGAEAGEIIGGRESRPHSRPYMAYLQIQSPAGQSRCGGFLVREDFVLTAAHCWGSNINVTLGAHNIQRREN
TQQHITARRAIRHPQYNQRTIQNDIMLLQLSRRVRRNRVNVPVALPRAQEGLRPGTLCTVAGWGRVSMRRGTDTLREVQLRVQR
DRQCLRIFGSYDPRRQICVGDRRERKAAFKGDSGGPLLCNNVAHGIVSYGKSSGVPPEVFTRVSSFLPWIRTTMRSFKLLDQME
TPL
SEQ ID No. 25: Nucleotide sequence encoding Homo sapiens Glyceraldehyde-3-phosphate
dehydrogenase (GAPDH), mRNA
NCBI Reference Sequence: NM_002046.5. The coding region ranges from nucleotide
189 to nucleotide 1196 (highlighted in bold). It is understood that the mRNA
corresponds to the sequence below (i.e. is identical to that sequence) with the
exception that the “t” (thymidine) residue is replaced by a “uracil” (u) residue.
ORIGIN
            1 gcctcaagac cttgggctgg gactggctga gcctggcggg aggcggggtc cgagtcaccg
          61 cctgccgccg cgcccccggt ttctataaat tgagcccgca gcctcccgct tcgctctctg
        121 ctcctcctgt tcgacagtca gccgcatctt cttttgcgtc gccagccgag ccacatcgct
        181 cagacaccat ggggaaggtg aaggtcggag tcaacggatt tggtcgtatt gggcgcctgg
        241 tcaccagggc tgcttttaac tctggtaaag tggatattgt tgccatcaat gaccccttca
        301 ttgacctcaa ctacatggtt tacatgttcc aatatgattc cacccatggc aaattccatg
        361 gcaccgtcaa ggctgagaac gggaagcttg tcatcaatgg aaatcccatc accatcttcc
        421 aggagcgaga tccctccaaa atcaagtggg gcgatgctgg cgctgagtac gtcgtggagt
        481 ccactggcgt cttcaccacc atggagaagg ctggggctca tttgcagggg ggagccaaaa
        541 gggtcatcat ctctgccccc tctgctgatg cccccatgtt cgtcatgggt gtgaaccatg
        601 agaagtatga caacagcctc aagatcatca gcaatgcctc ctgcaccacc aactgcttag
        661 cacccctggc caaggtcatc catgacaact ttggtatcgt ggaaggactc atgaccacag
        721 tccatgccat cactgccacc cagaagactg tggatggccc ctccgggaaa ctgtggcgtg
        781 atggccgcgg ggctctccag aacatcatcc ctgcctctac tggcgctgcc aaggctgtgg
        841 gcaaggtcat ccctgagctg aacgggaagc tcactggcat ggccttccgt gtccccactg
        901 ccaacgtgtc agtggtggac ctgacctgcc gtctagaaaa acctgccaaa tatgatgaca
        961 tcaagaaggt ggtgaagcag gcgtcggagg gccccctcaa gggcatcctg ggctacactg
      1021 agcaccaggt gttctcctct gacttcaaca gcgacaccca ctcctccacc tttgacgctg
      1081 gggctggcat tgccctcaac gaccactttg tcaagctcat ttcctggtat gacaacgaat
      1141 ttggctacag caacagggtg gtggacctca tggcccacat ggcctccaag gagtaagacc
      1201 cctggaccac cagccccagc aagagcacaa gaggaagaga gagaccctca ctgctgggga
      1261 gtccctgcca cactcagtcc cccaccacac tgaatctccc ctcctcacag ttgccatgta
      1321 gaccccttga agaggggagg ggcctaggga gccgcacctt gtcatgtacc atcaataaag
      1381 taccctgtgc tcaaccagtt aaaaaaaaaa aaaaaaaaaa aa
//
SEQ ID No. 26: Amino acid sequence of Homo sapiens Glyceraldehyde-3-phosphate
dehydrogenase (GAPDH), protein
UniProtKB/Swiss-Prot: G3P_HUMAN, P04406
MGKVKVGVNGFGRIGRLVTRAAFNSGKVDIVAINDPFIDLNYMVYMFQYDSTHGKFHGTVKAENGKLVINGNPITIFQERDPSK
IKWGDAGAEYVVESTGVFTTMEKAGAHLQGGAKRVIISAPSADAPMFVMGVNHEKYDNSLKIISNASCTTNCLAPAKVIHDNFG
IVEGLMTTVHAITATQKTVDGPSGKLWRDGRGALQNIIPASTGAAKAVGKVIPELNGKLTGMAFRVPTANVSVVDLTCRLEKPA
KYDDIKKVVKQASEGPLKGILGYTEHQVVSSDFNSDTHSSTFDAGAGIALNDHFVKLISWYDNEFGYSNRVVDLMAHMASKE
SEQ ID No. 27: Nucleotide sequence encoding Homo sapiens Hypoxanthine
phosphoribosyl transferase 1 (HPRT1), mRNA
NCBI Reference Sequence: NM_000194.2. The coding region ranges from nucleotide
168 to nucleotide 824 (highlighted in bold). It is understood that the mRNA
corresponds to the sequence below (i.e. is identical to that sequence) with the
exception that the “t” (thymidine) residue is replaced by a “uracil” (u) residue.
ORIGIN
            1 ggcggggcct gcttctcctc agcttcaggc ggctgcgacg agccctcagg cgaacctctc
          61 ggctttcccg cgcggcgccg cctcttgctg cgcctccgcc tcctcctctg ctccgccacc
        121 ggcttcctcc tcctgagcag tcagcccgcg cgccggccgg ctccgttatg gcgacccgca
        181 gccctggcgt cgtgattagt gatgatgaac caggttatga ccttgattta ttttgcatac
        241 ctaatcatta tgctgaggat ttggaaaggg tgtttattcc tcatggacta attatggaca
        301 ggactgaacg tcttgctcga gatgtgatga aggagatggg aggccatcac attgtagccc
        361 tctgtgtgct caaggggggc tataaattct ttgctgacct gctggattac atcaaagcac
        421 tgaatagaaa tagtgataga tccattccta tgactgtaga ttttatcaga ctgaagagct
        481 attgtaatga ccagtcaaca ggggacataa aagtaattgg tggagatgat ctctcaactt
        541 taactggaaa gaatgtcttg attgtggaag atataattga cactggcaaa acaatgcaga
        601 ctttgctttc cttggtcagg cagtataatc caaagatggt caaggtcgca agcttgctgg
        661 tgaaaaggac cccacgaagt gttggatata agccagactt tgttggattt gaaattccag
        721 acaagtttgt tgtaggatat gcccttgact ataatgaata cttcagggat ttgaatcatg
        781 tttgtgtcat tagtgaaact ggaaaagcaa aatacaaagc ctaagatgag agttcaagtt
        841 gagtttggaa acatctggag tcctattgac atcgccagta aaattatcaa tgttctagtt
        901 ctgtggccat ctgcttagta gagctttttg catgtatctt ctaagaattt tatctgtttt
        961 gtactttaga aatgtcagtt gctgcattcc taaactgttt atttgcacta tgagcctata
      1021 gactatcagt tccctttggg cggattgttg tttaacttgt aaatgaaaaa attctcttaa
      1081 accacagcac tattgagtga aacattgaac tcatatctgt aagaaataaa gagaagatat
      1141 attagttttt taattggtat tttaattttt atatatgcag gaaagaatag aagtgattga
      1201 atattgttaa ttataccacc gtgtgttaga aaagtaagaa gcagtcaatt ttcacatcaa
      1261 agacagcatc taagaagttt tgttctgtcc tggaattatt ttagtagtgt ttcagtaatg
      1321 ttgactgtat tttccaactt gttcaaatta ttaccagtga atctttgtca gcagttccct
      1381 tttaaatgca aatcaataaa ttcccaaaaa tttaaaaaaa aaaaaaaaaa aaaaa
//
SEQ ID No.: 28 Amino acid sequence of Homo sapiens Hypoxanthine phosphoribosyl
transferase 1 (HPRT1), protein
UnitProtKB/Swiss-Prot: HPRT_HUMAN, P00492
MATRSPGVVISDDEPGYDLDLFCIPNHYAEDLERVFIOPHGLIMDRTERLARDVMKEMGGHHIVALCVLKGGYKFFADLLDYIK
ALNRNSDRSIPMTVDFIRLKSYCNDQSTGDIKVIGGDGLSTLTGKNVLIVEDIIDTGKTMQTLLSLVRQYNPKMVKVASLLVKR
TPRSVGYKPDFVGFEIPDKFVVGYALDYNEYFRDLNHVCVISETGKAKYKA
```

All references cited herein are fully incorporated by reference. Having now fully described the invention, it will be understood by a person skilled in the art that the invention may be practiced within a wide and equivalent range of conditions, parameters and the like, without affecting the spirit or scope of the invention or any embodiment thereof.

1. A method for monitoring the response to treatment with an LSD1 inhibitor in a subject suffering from leukemia, said method comprising determining the level of one or more of the markers VCAN, S100A12, ITGAM, LY96, ANXA2, CD86, GPR65, CRISP9, LYZ and VIM, in a sample from said subject, wherein an increased level of one or more of the markers VCAN, S100A12, ITGAM, LY96, ANXA2, CD86, GPR65, CRISP9, LYZ and VIM compared to a control is indicative for response to treatment.

2. A method for the identification of a responding subject to treatment with an LSD1 inhibitor, said method comprising determining the level of one or more of the markers VCAN, S100A12, ITGAM, LY96, ANXA2, CD86, GPR65, CRISP9, LYZ and VIM in a sample from a subject suffering from leukemia, wherein an increased level of one or more of the markers VCAN, S100A12, ITGAM, LY96, ANXA2, CD86, GPR65, CRISP9, LYZ and VIM compared to a control is indicative for a responding subject.

3. A method of determining whether a proliferative diseased cell is responsive to treatment with an LSD1 inhibitor,
said method comprising determining the level of one or more of the markers VCAN, S100A12, ITGAM, LY96, ANXA2, CD86, GPR65, CRISP9, LYZ and VIM in a sample from a subject suffering from leukemia, wherein an increased level of one or more of the markers VCAN, S100A12, ITGAM, LY96, ANXA2, CD86, GPR65, CRISP9, LYZ and VIM compared to a control is indicative for a responsive proliferative diseased cell.

4. The method of any one of claims 1 to 3, wherein said leukemia is myeloid leukemia.

5. The method of claim 4, wherein said myeloid leukemia is acute myeloid leukemia (AML).

6. The method of any one of claims 1 to 5, wherein said AML is acute myelomonocytic leukemia, acute monoblastic leukemia or acute monocytic leukemia.

7. The method of any one of claims 1 to 5 wherein said AML is AML subtype M4 or M5.

8. The method of any one of claims 1 to 7, wherein the level of 2 or more, 3 or more, 4 or more, 5 or more, 6 or more, 7 or more, 8 or more, or 9, or 10 of the markers VCAN, S100A12, ITGAM, LY96, ANXA2, CD86, GPR65, CRISP9, LYZ and VIM is determined.

9. The method of any one of claims 1 to 7, wherein the level of one or more, 2 or more, 3 or more, 4 or more, 5 or more, 6 or more, 7 or more, 8 or 9, of the markers VCAN, S100A12, ITGAM, LY96, ANXA2, CD86, GPR65, CRISP9, and LYZ is determined.

10. The method of any one of claims 1 to 9, wherein said sample is to be obtained from said subject after the initiation of the treatment with said LSD1 inhibitor.

11. The method of claim 10, wherein said sample is to be obtained from said subject at day 3 or at a subsequent day after the initiation of the treatment with said LSD1 inhibitor.

12. The method of claim 11, wherein said sample is to be obtained from said subject at any one of days 3 to 26 days after the initiation of the treatment with said LSD1 inhibitor.

13. The method of any one of claims 1 to 12, wherein said subject is a human.

14. The method of any one of claims 1 to 13, wherein said level of one or more of the markers VCAN, S100A12, ITGAM, LY96, ANXA2, CD86, GPR65, CRISP9, LYZ and VIM, is at least 1.3-fold, preferably at least 2-fold increased in comparison to a control.

15. The method of any one of claims 1 to 14, wherein the control for said marker is the level of said marker determined in a sample of said same subject prior to the initiation of treatment with said LSD1 inhibitor.

16. The method of any one of claims 1 to 15, wherein said level of said one or more of the markers VCAN, S100A12, ITGAM, LY96, ANXA2, CD86, GPR65, CRISP9, LYZ and VIM is the expression level.

17. The method of claim 16, wherein said expression level is the mRNA expression level.

18. The method of claim 17, wherein the mRNA expression level is assessed by PCR, in situ hybridization, Whole Transcriptome Sequencing (RNAseq), nanopore sequencing, digital gene expresion or micro-array analysis.

19. The method of claim 18, wherein said PCR is quantitative PCR or RealTime PCR, preferably quantitative RealTime PCR (qPCR).

20. The method of claim 16, wherein said expression level is the protein expression level.

21. The method of claim 20, wherein said protein expression level is assessed by immunoassay, gel- or blot-based methods, IHC, mass spectrometry, flow cytometry, FACS or protein activity assay.

22. The method of any one of claims 16 to 21, wherein the expression level is normalized to the expression level of an endogenous gene.

23. The method of claim 22, wherein said endogenous gene is GADPH or HPRT1.

24. The method of claim 23, wherein said endogenous gene is HPRT1.

25. The method of any one of claims 1 to 24, wherein if the level of one or more of the markers VCAN, S100A12, ITGAM, LY96, ANXA2, CD86, GPR65, CRISP9, LYZ and VIM is not increased compared to a control, the treatment with said LSD1 inhibitor is adapted.

26. The method of claim 25, wherein said adaption of the treatment with said LSD1 inhibitor comprises increasing the dose of said LSD1 inhibitor.

27. The method of any one of claims 1 to 26, wherein said sample is a blood sample, in particular a peripheral blood sample.

28. The method of any one of claims 1 to 27, wherein said LSD1 inhibitor is a 2-(hetero)arylcyclopropylamino compound.

29. The method of any of claims 1 to 28, wherein said LSD1 inhibitor is a compound disclosed in WO2010/043721, WO2010/084160, WO2011/035941, WO2011/042217, WO2011/131697, WO2012/013727, WO2012/013728, WO2012/045883, WO2013/057320, WO2013/057322, WO2012/135113, WO2013/022047, WO2014/058071, WO2010/143582, US2010-0324147, WO2011/131576, WO2014/084298, WO2014/086790, WO2014/164867, WO2014/194280, WO2015/021128, WO2015/123465, WO2015/123437, WO2015/123424, WO2015/123408, WO2015/156417, WO2015/181380, WO2016/123387 or WO2016/130952.

30. The method of any of claims 1 to 29, wherein said LSD1 inhibitor is (trans)-N1-((1R,2S)-2-phenylcyclopropyl)cyclohexane-1,4-diamine or a pharmaceutically acceptable salt or solvate thereof.

31. The method of any of claims 1 to 30, wherein said LSD1 inhibitor is (trans)-N1-((1R,2S)-2-phenylcyclopropyl)cyclohexane-1,4-diamine bis-hydrochloride.

32. The method of any one of claims 1 to 31, wherein said method is an in vitro method.

33. A method of treating a subject suffering from leukemia with an LSD1 inhibitor, wherein the subject is identified as a responder to treatment with an LSD1 inhibitor according to any one of claims 2 and 4 to 32.

34. LSD1 inhibitor for use in treating a subject suffering from leukemia, wherein the subject is identified as a responder to treatment with an LSD1 inhibitor according to any one of claims 2 and 4 to 32

35. A kit for use in carrying out the method of any one of claims of 1 to 33, comprising means for determining the level of one or more of the markers VCAN, S100A12, ITGAM, LY96, ANXA2, CD86, GPR65, CRISP9, LYZ and VIM.

36. A method for assessing whether a subject is at risk of developing a differentiation syndrome (DS), said method comprising determining the level of one or more of the markers VCAN, S100A12, ITGAM, LY96, ANXA2, CD86, GPR65, CRISP9, LYZ and VIM, in a sample from said subject, wherein an increased level of one or more of the markers VCAN, S100A12, ITGAM, LY96, ANXA2, CD86, GPR65, CRISP9, LYZ and VIM compared to a control is indicative for an increased risk of developing a differentiation syndrome (DS).

37. The method of claim 36, which comprises determining the level of one or more of the markers VCAN and S100A12.


SEQUENCE LISTING (28 sequences)

SEQ ID NO: 1 - RNA, 466 nt, Homo sapiens
  Nucleotide sequence encoding Homo sapiens S100 calcium binding protein A12 (S100A12)
accactgctggctttttgctgtagctccacattcctgtgcattgaggggttaacattaggctgggaagatgacaaaacttgaagagcatctggagggaattgtcaatatcttccaccaatactcagttcggaaggggcattttgacaccctctctaagggtgagctgaagcagctgcttacaaaggagcttgcaaacaccatcaagaatatcaaagataaagctgtcattgatgaaatattccaaggcctggatgctaatcaagatgaacaggtcgactttcaagaattcatatccctggtagccattgcgctgaaggctgcccattaccacacccacaaagagtaggtagctctctgaaggctttttacccagcaatgtcctcaatgagggtcttttctttccctcaccaaaacccagccttgcccgtggggagtaagagttaataaacacactcacgaaaagtt

SEQ ID NO: 2 - protein, 92 aa, Homo sapiens
  Amino acid sequence of Homo sapiens S100 calcium binding protein A12 (S100A12)
MTKLEEHLEGIVNIFHQYSVRKGHFDTLSKGELKQLLTKELANTIKNIKDKAVIDEIFQGLDANQDEQVDFQEFISLVAIALKAAHYHTHKE

SEQ ID NO: 3 - RNA, 4193 nt, Homo sapiens
  Nucleotide sequence encoding Homo sapiens Versican (VCAN)
cttcttctcgctgagtctcctcctcggctctgacggtacagtgatataatgatgatgggtgtcacaacccgcatttgaacttgcaggcgagctgccccgagcctttctggggaagaactccaggcgtgcggacgcaacagccgagaacattaggtgttgtggacaggagctgggaccaagatcttcggccagccccgcatcctcccgcatcttccagcaccgtcccgcaccctccgcatccttccccgggccaccacgcttcctatgtgacccgcctgggcaacgccgaacccagtcgcgcagcgctgcagtgaattttccccccaaactgcaataagccgccttccaaggccaagatgttcataaatataaagagcatcttatggatgtgttcaaccttaatagtaacccatgcgctacataaagtcaaagtgggaaaaagcccaccggtgaggggctccctctctggaaaagtcagcctaccttgtcatttttcaacgatgcctactttgccacccagttacaacaccagtgaatttctccgcatcaaatggtctaagattgaagtggacaaaaatggaaaagatttgaaagagactactgtccttgtggcccaaaatggaaatatcaagattggtcaggactacaaagggagagtgtctgtgcccacacatcccgaggctgtgggcgatgcctccctcactgtggtcaagctgctggcaagtgatgcgggtctttaccgctgtgacgtcatgtacgggattgaagacacacaagacacggtgtcactgactgtggatggggttgtgtttcactacagggcggcaaccagcaggtacacactgaattttgaggctgctcagaaggcttgtttggacgttggggcagtcatagcaactccagagcagctctttgctgcctatgaagatggatttgagcagtgtgacgcaggctggctggctgatcagactgtcagatatcccatccgggctcccagagtaggctgttatggagataagatgggaaaggcaggagtcaggacttatggattccgttctccccaggaaacttacgatgtgtattgttatgtggatcatctggatggtgatgtgttccacctcactgtccccagtaaattcaccttcgaggaggctgcaaaagagtgtgaaaaccaggatgccaggctggcaacagtgggggaactccaggcggcatggaggaacggctttgaccagtgcgattacgggtggctgtcggatgccagcgtgcgccaccctgtgactgtggccagggcccagtgtggaggtggtctacttggggtgagaaccctgtatcgttttgagaaccagacaggcttccctccccctgatagcagatttgatgcctactgctttaaacgacctgatcgctgcaaaatgaacccgtgccttaacggaggcacctgttatcctactgaaacttcctacgtatgcacctgtgtgccaggatacagcggagaccagtgtgaacttgattttgatgaatgtcactctaatccctgtcgtaatggagccacttgtgttgatggttttaacacattcaggtgcctctgccttccaagttatgttggtgcactttgtgagcaagataccgagacatgtgactatggctggcacaaattccaagggcagtgctacaaatactttgcccatcgacgcacatgggatgcagctgaacgggaatgccgtctgcagggtgcccatctcacaagcatcctgtctcacgaagaacaaatgtttgttaatcgtgtgggccatgattatcagtggataggcctcaatgacaagatgtttgagcatgacttccgttggactgatggcagcacactgcaatacgagaattggagacccaaccagccagacagcttcttttctgctggagaagactgtgttgtaatcatttggcatgagaatggccagtggaatgatgttccctgcaattaccatctcacctatacgtgcaagaaaggaacagtcgcttgcggccagccccctgttgtagaaaatgccaagacctttggaaagatgaaacctcgttatgaaatcaactccctgattagataccactgcaaagatggtttcattcaacgtcaccttccaactatccggtgcttaggaaatggaagatgggctatacctaaaattacctgcatgaacccatctgcataccaaaggacttattctatgaaatactttaaaaattcctcatcagcaaaggacaattcaataaatacatccaaacatgatcatcgttggagccggaggtggcaggagtcgaggcgctgatccctaaaatggcgaacatgtgttttcatcatttcagccaaagtcctaacttcctgtgcctttcctatcacctcgagaagtaattatcagttggtttggatttttggaccaccgttcagtcattttgggttgccgtgctcccaaaacattttaaatgaaagtattggcattcaaaaagacagcagacaaaatgaaagaaaatgagagcagaaagtaagcatttccagcctatctaatttctttagttttctatttgcctccagtgcagtccatttcctaatgtataccagcctactgtactatttaaaatgctcaatttcagcaccgatggccatgtaaataagatgatttaatgttgattttaatcctgtatataaaataaaaagtcacaatgagtttgggcatatttaatgatgattatggagccttagaggtctttaatcattggttcggctgcttttatgtagtttaggctggaaatggtttcacttgctctttgactgtcagcaagactgaagatggcttttcctggacagctagaaaacacaaaatcttgtaggtcattgcacctatctcagccataggtgcagtttgcttctacatgatgctaaaggctgcgaatgggatcctgatggaactaaggactccaatgtcgaactcttctttgctgcattcctttttcttcacttacaagaaaggcctgaatggaggacttttctgtaaccaggaacattttttaggggtcaaagtgctaataattaactcaaccaggtctactttttaatggctttcataacactaactcataaggttaccgatcaatgcatttcatacggatatagacctagggctctggagggtgggggattgttaaaacacatgcaaaaaaaaaaaaaaaaaaaaaaaaagaaattttgtatatataaccattttaatcttttataaagttttgaatgttcatgtatgaatgctgcagctgtgaagcatacataaataaatgaagtaagccatactgatttaatttattggatgttattttccctaagacctgaaaatgaacatagtatgctagttatttttcagtgttagccttttactttcctcacacaatttggaatcatataatataggtactttgtccctgattaaataatgtgacggatagaatgcatcaagtgtttattatgaaaagagtggaaaagtatatagcttttagcaaaaggtgtttgcccattctaagaaatgagcgaatatatagaaatagtgtgggcatttcttcctgttaggtggagtgtatgtgttgacatttctccccatctcttcccactctgttttctccccattatttgaataaagtgactgctgaagatgactttgaatccttatccacttaatttaatgtttaaagaaaaacctgtaatggaaagtaagactccttccctaatttcagtttagagcaacttgaagaagagtagacaaaaaataaaatgcacatagaaaaagagaaaaagggcacaaagggattggcccaatattgattctttttttataaaacctcctttggcttagaaggaatgactctagctacaataatacacagtatgtttaagcaggttcccttggttgttgcattaaatgtaatccacctttaggtattttagagcacagaacaacactgtgttgatctagtaggtttctatttttcctttctctttacaatgcacataatactttcctgtatttatatcataacgtgtatagtgtaaaatgtgaatgactttttttgtgaatgaaaatctaaaatctttgtaactttttatatctgcttttgtttcaccaaagaaacctaaaatccttcttttactacac

SEQ ID NO: 4 - protein, 655 aa, Homo sapiens
  Amino acid sequence of Homo sapiens Versican (VCAN)
MFINIKSILWMCSTLIVTHALHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKRPDRCKMNPCLNGGTCYPTETSYVCTCVPGYSGDQCELDFDECHSNPCRNGATCVDGFNTFRCLCLPSYVGALCEQDTETCDYGWHKFQGQCYKYFAHRRTWDAAERECRLQGAHLTSILSHEEQMFVNRVGHDYQWIGLNDKMFEHDFRWTDGSTLQYENWRPNQPDSFFSAGEDCVVIIWHENGQWNDVPCNYHLTYTCKKGTVACGQPPVVENAKTFGKMKPRYEINSLIRYHCKDGFIQRHLPTIRCLGNGRWAIPKITCMNPSAYQRTYSMKYFKNSSSAKDNSINTSKHDHRWSRRWQESRR

SEQ ID NO: 5 - RNA, 4745 nt, Homo sapiens
  Nucleotide sequence encoding Homo sapiens Integrin subunit alpha M (ITGAM)
ttttctgcccttctttgctttggtggcttccttgtggttcctcagtggtgcctgcaacccctggttcacctccttccaggttctggctccttccagccatggctctcagagtccttctgttaacagccttgaccttatgtcatgggttcaacttggacactgaaaacgcaatgaccttccaagagaacgcaaggggcttcgggcagagcgtggtccagcttcagggatccagggtggtggttggagccccccaggagatagtggctgccaaccaaaggggcagcctctaccagtgcgactacagcacaggctcatgcgagcccatccgcctgcaggtccccgtggaggccgtgaacatgtccctgggcctgtccctggcagccaccaccagcccccctcagctgctggcctgtggtcccaccgtgcaccagacttgcagtgagaacacgtatgtgaaagggctctgcttcctgtttggatccaacctacggcagcagccccagaagttcccagaggccctccgagggtgtcctcaagaggatagtgacattgccttcttgattgatggctctggtagcatcatcccacatgactttcggcggatgaaggagtttgtctcaactgtgatggagcaattaaaaaagtccaaaaccttgttctctttgatgcagtactctgaagaattccggattcactttaccttcaaagagttccagaacaaccctaacccaagatcactggtgaagccaataacgcagctgcttgggcggacacacacggccacgggcatccgcaaagtggtacgagagctgtttaacatcaccaacggagcccgaaagaatgcctttaagatcctagttgtcatcacggatggagaaaagtttggcgatcccttgggatatgaggatgtcatccctgaggcagacagagagggagtcattcgctacgtcattggggtgggagatgccttccgcagtgagaaatcccgccaagagcttaataccatcgcatccaagccgcctcgtgatcacgtgttccaggtgaataactttgaggctctgaagaccattcagaaccagcttcgggagaagatctttgcgatcgagggtactcagacaggaagtagcagctcctttgagcatgagatgtctcaggaaggcttcagcgctgccatcacctctaatggccccttgctgagcactgtggggagctatgactgggctggtggagtctttctatatacatcaaaggagaaaagcaccttcatcaacatgaccagagtggattcagacatgaatgatgcttacttgggttatgctgccgccatcatcttacggaaccgggtgcaaagcctggttctgggggcacctcgatatcagcacatcggcctggtagcgatgttcaggcagaacactggcatgtgggagtccaacgctaatgtcaagggcacccagatcggcgcctacttcggggcctccctctgctccgtggacgtggacagcaacggcagcaccgacctggtcctcatcggggccccccattactacgagcagacccgagggggccaggtgtccgtgtgccccttgcccagggggcagagggctcggtggcagtgtgatgctgttctctacggggagcagggccaaccctggggccgctttggggcagccctaacagtgctgggggacgtaaatggggacaagctgacggacgtggccattggggccccaggagaggaggacaaccggggtgctgtttacctgtttcacggaacctcaggatctggcatcagcccctcccatagccagcggatagcaggctccaagctctctcccaggctccagtattttggtcagtcactgagtgggggccaggacctcacaatggatggactggtagacctgactgtaggagcccaggggcacgtgctgctgctcaggtcccagccagtactgagagtcaaggcaatcatggagttcaatcccagggaagtggcaaggaatgtatttgagtgtaatgatcaggtggtgaaaggcaaggaagccggagaggtcagagtctgcctccatgtccagaagagcacacgggatcggctaagagaaggacagatccagagtgttgtgacttatgacctggctctggactccggccgcccacattcccgcgccgtcttcaatgagacaaagaacagcacacgcagacagacacaggtcttggggctgacccagacttgtgagaccctgaaactacagttgccgaattgcatcgaggacccagtgagccccattgtgctgcgcctgaacttctctctggtgggaacgccattgtctgctttcgggaacctccggccagtgctggcggaggatgctcagagactcttcacagccttgtttccctttgagaagaattgtggcaatgacaacatctgccaggatgacctcagcatcaccttcagtttcatgagcctggactgcctcgtggtgggtgggccccgggagttcaacgtgacagtgactgtgagaaatgatggtgaggactcctacaggacacaggtcaccttcttcttcccgcttgacctgtcctaccggaaggtgtccacgctccagaaccagcgctcacagcgatcctggcgcctggcctgtgagtctgcctcctccaccgaagtgtctggggccttgaagagcaccagctgcagcataaaccaccccatcttcccggaaaactcagaggtcacctttaatatcacgtttgatgtagactctaaggcttcccttggaaacaaactgctcctcaaggccaatgtgaccagtgagaacaacatgcccagaaccaacaaaaccgaattccaactggagctgccggtgaaatatgctgtctacatggtggtcaccagccatggggtctccactaaatatctcaacttcacggcctcagagaataccagtcgggtcatgcagcatcaatatcaggtcagcaacctggggcagaggagcctccccatcagcctggtgttcttggtgcccgtccggctgaaccagactgtcatatgggaccgcccccaggtcaccttctccgagaacctctcgagtacgtgccacaccaaggagcgcttgccctctcactccgactttctggctgagcttcggaaggcccccgtggtgaactgctccatcgctgtctgccagagaatccagtgtgacatcccgttctttggcatccaggaagaattcaatgctaccctcaaaggcaacctctcgtttgactggtacatcaagacctcgcataaccacctcctgatcgtgagcacagctgagatcttgtttaacgattccgtgttcaccctgctgccgggacagggggcgtttgtgaggtcccagacggagaccaaagtggagccgttcgaggtccccaaccccctgccgctcatcgtgggcagctctgtcgggggactgctgctcctggccctcatcaccgccgcgctgtacaagctcggcttcttcaagcggcaatacaaggacatgatgagtgaagggggtcccccgggggccgaaccccagtagcggctccttcccgacagagctgcctctcggtggccagcaggactctgcccagaccacacgtagcccccaggctgctggacacgtcggacagcgaagtatccccgacaggacgggcttgggcttccatttgtgtgtgtgcaagtgtgtatgtgcgtgtgtgcaagtgtctgtgtgcaagtgtgtgcacatgtgtgcgtgtgcgtgcatgtgcacttgcacgcccatgtgtgagtgtgtgcaagtatgtgagtgtgtccaagtgtgtgtgcgtgtgtccatgtgtgtgcaagtgtgtgcatgtgtgcgagtgtgtgcatgtgtgtgctcaggggcgtgtggctcacgtgtgtgactcagatgtctctggcgtgtgggtaggtgacggcagcgtagcctctccggcagaagggaactgcctgggctcccttgtgcgtgggtgaagccgctgctgggttttcctccgggagaggggacggtcaatcctgtgggtgaagacagagggaaacacagcagcttctctccactgaaagaagtgggacttcccgtcgcctgcgagcctgcggcctgctggagcctgcgcagcttggatggagactccatgagaagccgtgggtggaaccaggaacctcctccacaccagcgctgatgcccaataaagatgcccactgaggaatgatgaagcttcctttctggattcatttattatttcaatgtgactttaattttttggatggataagcttgtctatggtacaaaaatcacaaggcattcaagtgtacagtgaaaagtctccctttccagatattcaagtcacctccttaaaggtagtcaagattgtgttttgaggtttccttcagacagattccaggcgatgtgcaagtgtatgcacgtgtgcacacacaccacacatacacacacacaagcttttttacacaaatggtagcatactttatattggtctgtatcttgctttttttcaccaatatttctcagacatcggttcatattaagacataaattactttttcattcttttataccgctgcatagtattccattgtgtgagtgtaccataatgtatttaaccagtcttcttttgatatactattttcattctcttgttattgcatcaatgctgagttaataaatcaaatatatgtcatttttgcatatatgtaaggataa

SEQ ID NO: 6 - protein, 1152 aa, Homo sapiens
  Amino acid sequence of Homo sapiens Integrin subunit alpha M (ITGAM)
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 7 - RNA, 642 nt, Homo sapiens
  Nucleotide sequence encoding Homo sapiens Lymphocyte antigen 96 (Ly96)
agaaatcatgtgactgatgactaagttaaatcttttctgcttactgaaaaggaagagtctgatgattagttactgatcctctttgcatttgtaaagctttggagatattgaatcatgttaccatttctgtttttttccaccctgttttcttccatatttactgaagctcagaagcagtattgggtctgcaactcatccgatgcaagtatttcatacacctactgtgataaaatgcaatacccaatttcaattaatgttaacccctgtatagaattgaaaggatccaaaggattattgcacattttctacattccaaggagagatttaaagcaattatatttcaatctctatataactgtcaacaccatgaatcttccaaagcgcaaagaagttatttgccgaggatctgatgacgattactctttttgcagagctctgaagggagagactgtgaatacaacaatatcattctccttcaagggaataaaattttctaagggaaaatacaaatgtgttgttgaagctatttctgggagcccagaagaaatgctcttttgcttggagtttgtcatcctacaccaacctaattcaaattagaataaattgagtatttaaaaaaaaaaaaaaaaaaaaaaaaaaaaa

SEQ ID NO: 8 - protein, 160 aa, Homo sapiens [sequence omitted]

SEQ ID NO: 9 - RNA, 1632 nt, Homo sapiens
  Nucleotide sequence encoding Homo sapiens Annexin A2 (ANXA2)
gctcagcatttggggacgctctcagctctcggcgcacggcccaggtaagcggggcgcgccctgcccgcccgcgatgggccgccagctagcggggtgtggagacgctgggaagaaggcttccttcaaaatgtctactgttcacgaaatcctgtgcaagctcagcttggagggtgatcactctacacccccaagtgcatatgggtctgtcaaagcctatactaactttgatgctgagcgggatgctttgaacattgaaacagccatcaagaccaaaggtgtggatgaggtcaccattgtcaacattttgaccaaccgcagcaatgcacagagacaggatattgccttcgcctaccagagaaggaccaaaaaggaacttgcatcagcactgaagtcagccttatctggccacctggagacggtgattttgggcctattgaagacacctgctcagtatgacgcttctgagctaaaagcttccatgaaggggctgggaaccgacgaggactctctcattgagatcatctgctccagaaccaaccaggagctgcaggaaattaacagagtctacaaggaaatgtacaagactgatctggagaaggacattatttcggacacatctggtgacttccgcaagctgatggttgccctggcaaagggtagaagagcagaggatggctctgtcattgattatgaactgattgaccaagatgctcgggatctctatgacgctggagtgaagaggaaaggaactgatgttcccaagtggatcagcatcatgaccgagcggagcgtgccccacctccagaaagtatttgataggtacaagagttacagcccttatgacatgttggaaagcatcaggaaagaggttaaaggagacctggaaaatgctttcctgaacctggttcagtgcattcagaacaagcccctgtattttgctgatcggctgtatgactccatgaagggcaaggggacgcgagataaggtcctgatcagaatcatggtctcccgcagtgaagtggacatgttgaaaattaggtctgaattcaagagaaagtacggcaagtccctgtactattatatccagcaagacactaagggcgactaccagaaagcgctgctgtacctgtgtggtggagatgactgaagcccgacacggcctgagcgtccagaaatggtgctcaccatgcttccagctaacaggtctagaaaaccagcttgcgaataacagtccccgtggccatccctgtgagggtgacgttagcattacccccaacctcattttagttgcctaagcattgcctggccttcctgtctagtctctcctgtaagccaaagaaatgaacattccaaggagttggaagtgaagtctatgatgtgaaacactttgcctcctgtgtactgtgtcataaacagatgaataaactgaatttgtactttagaaacacgtactttgtggccctgctttcaactgaattgtttgaaaattaaacgtgcttggggttcagctggtgaggctgtccctgtaggaagaaagctctgggactgagctgtacagtatggttgcccctatccaagtgtcgctatttaagttaaatttaaatgaaataaaataaaataaaatcaaaaaaa

SEQ ID NO: 10 - protein, 339 aa, Homo sapiens
  Amino acid sequence of Homo sapiens Annexin A2 (ANXA2)
MSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 11 - RNA, 2399 nt, Homo sapiens
  Nucleotide sequence encoding Homo sapiens CD86 Molecule (CD86)
agtcattgccgaggaaggcttgcacagggtgaaagctttgcttctctgctgctgtaacagggactagcacagacacacggatgagtggggtcatttccagatattaggtcacagcagaagcagccaaaatggatccccagtgcactatgggactgagtaacattctctttgtgatggccttcctgctctctgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtattatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaagatctgatgaagcccagcgtgtttttaaaagttcgaagacatcttcatgcgacaaaagtgatacatgtttttaattaaagagtaaagcccatacaagtattcattttttctaccctttcctttgtaagttcctgggcaacctttttgatttcttccagaaggcaaaaagacattaccatgagtaataagggggctccaggactccctctaagtggaatagcctccctgtaactccagctctgctccgtatgccaagaggagactttaattctcttactgcttcttttcacttcagagcacacttatgggccaagcccagcttaatggctcatgacctggaaataaaatttaggaccaatacctcctccagatcagattcttctcttaatttcatagattgtgttttttttttaaatagacctctcaatttctggaaaactgccttttatctgcccagaattctaagctggtgccccactgaattttgtgtacctgtgactaaacaactacctcctcagtctgggtgggacttatgtatttatgaccttatagtgttaatatcttgaaacatagagatctatgtactgtaatagtgtgattactatgctctagagaaaagtctacccctgctaaggagttctcatccctctgtcagggtcagtaaggaaaacggtggcctagggtacaggcaacaatgagcagaccaacctaaatttggggaaattaggagaggcagagatagaacctggagccacttctatctgggctgttgctaatattgaggaggcttgccccacccaacaagccatagtggagagaactgaataaacaggaaaatgccagagcttgtgaaccctgtttctcttgaagaactgactagtgagatggcctggggaagctgtgaaagaaccaaaagagatcacaatactcaaaagagagagagagagaaaaaagagagatcttgatccacagaaatacatgaaatgtctggtctgtccaccccatcaacaagtcttgaaacaagcaacagatggatagtctgtccaaatggacataagacagacagcagtttccctggtggtcagggaggggttttggtgatacccaagttattgggatgtcatcttcctggaagcagagctggggagggagagccatcaccttgataatgggatgaatggaaggaggcttaggactttccactcctggctgagagaggaagagctgcaacggaattaggaagaccaagacacagatcacccggggcttacttagcctacagatgtcctacgggaacgtgggctggcccagcatagggctagcaaatttgagttggatgattgtttttgctcaaggcaaccagaggaaacttgcatacagagacagatatactgggagaaatgactttgaaaacctggctctaaggtgggatcactaagggatggggcagtctctgcccaaacataaagagaactctggggagcctgagccacaaaaatgttcctttattttatgtaaaccctcaagggttatagactgccatgctagacaagcttgtccatgtaatattcccatgtttttaccctgcccctgccttgattagactcctagcacctggctagtttctaacatgttttgtgcagcacagtttttaataaatgcttgttacattcatttaaaaaaaaaaaaaa

SEQ ID NO: 12 - protein, 329 aa, Homo sapiens
  Amino acid sequence of Homo sapiens CD86 Molecule (CD86)
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 13 - RNA, 4522 nt, Homo sapiens
  Nucleotide sequence encoding Homo sapiens G protein-coupled receptor 65 (GPR65)
cctgtcccctcagcagtgttggtttctcttcttgacttgatgcaggcacagatttatcaagctcctcagtcaacaaacacatcaccggaagaaatatggaaggaaaggaattttaaaaggaaataccaatctctgtgcaaacaaagccttgtatattcatgtttgcaccaatctactgtgagatttatgaagaaaaacaaattgcggacaactctctatgtacacttacaaatgcctcagttgatgcttgtgggctgtttgtcagcgttctgtgataatgaacacatggacttctgtttattaaattcagttgacccctttagccaattgccaggagcctggatttttacttccaactgctgatatctgtgtaaaaattgatctacatccaccctttaaaagcattgatgaattaattagaactttagacaacaaagaaaaattgaaaaagaattctcagtaaaagcgaattcgatgttcaaaacaaactacaaagagacaagacttctctgtttactttctaagaactaatataattgctaccttaaaaaggaaaaaatgaacagcacatgtattgaagaacagcatgacctggatcactatttgtttcccattgtttacatctttgtgattatagtcagcattccagccaatattggatctctgtgtgtgtctttcctgcaagcaaagaaggaaagtgaactaggaatttacctcttcagtttgtcactatcagatttactctatgcattaactctccctttatggattgattatacctggaataaagacaactggactttctctcctgccttgtgcaaagggagtgcttttctcatgtacatgaatttttacagcagcacagcattcctcacctgcattgccgttgatcggtatttggctgttgtctaccctttgaagttttttttcctaaggacaagaagatttgcactcatggtcagcctgtccatctggatattggaaaccatcttcaatgctgtcatgttgtgggaagatgaaacagttgttgaatattgcgatgccgaaaagtctaattttactttatgctatgacaaataccctttagagaaatggcaaatcaacctcaacttgttcaggacgtgtacaggctatgcaatacctttggtcaccatcctgatctgcaaccggaaagtctaccaagctgtgcggcacaataaagccacggaaaacaaggaaaagaagagaatcataaaactacttgtcagcatcacagttacttttgtcttatgctttactccctttcatgtgatgttgctgattcgctgcattttagagcatgctgtgaacttcgaagaccacagcaattctgggaagcgaacttacacaatgtatagaatcacggttgcattaacaagtttaaattgtgttgctgatccaattctgtactgttttgtaaccgaaacaggaagatatgatatgtggaatatattaaaattctgcactgggaggtgtaatacatcacaaagacaaagaaaacgcatactttctgtgtctacaaaagatactatggaattagaggtccttgagtagaaccaaggatgttttgaagggaagggaagtttaagttatgcattattatatcatcaagattacattttgaaaaggaaatctagcatgtgaggggactaagtgttctcagagtgatgttttaatccagtccaataaaaatatcttaaaactgcattgtacagctccctccctgcgttttattaaatgatgtatattaaacaaagatcaatattttcttaatgactcagggtctttattgttaatgccaattgtttttgtatctgtgctataatcccttagagtcagtaaagtatgtaggggactgtttcttcctttgtgtctgggtttatgatttttctcactctttctttggactccagggtgtcagccatcaggtctcctaattttgtgtaccggtctccaacaaccccagctactgaatactgcttctaatctcctcattcattaacaaatctttatttttttatcttgtataaaataactgctttattgacacaaaatttacataacttaaaattcaactttgtattgtgtacaattcagtgattttttgtatattcacagagctgtgcaaccatcaccacactcaaaaaattttcatcacccaccaaagaaatcttatactcttagcagtcgctccctgctctcccgtccatgccagttattaatttactttctgtctctaaggattttcattactctgaacatttcatataaatagaattatacaatatgtggcctactgtgacgtatttcacttagtataatggtttcaagttttatccatgtgtagaatgtatcagcacttcatttctttttatggcctgatagtattctgttgcatggttatactccattttgtttatctaatcacttggcttcattaacaaatatttattgaatccattccataaactaggttttgagttaagtactggggctatgaaagaaatggtctcatgaagcctcacgaagtttacattagttcaaaagcctagtcaccgagcttgaaagatttctatataaaggaaaaggaaataggctctgagttttattttgatctctttttaatttataactgggtataacatagctgaaattaccagaagtttaatgcatagacaaataaatagttctattatatctttctttttggacttagaatgttagaatattttgagagttctttttttttttttttttgagtcagagtcttgctctgtaatccaggctagagtgtagtggtgcgatctccactcactgcagcctccacctcccaggttcaagcgattctcctgcctcagcctcccaagtagctgggattacaggcacccaccaccatgcccagctaatttttgtatttttagtagagacggggtttcaccatgttgcacaggctggtctcaatcgaactcctgacctcaagtgatcatcccacctaggtctcccaaagtgctgagatgacaggcgtgagccaccatgcctggcaaagagagtcttgatacaacatattcttttgaatcctcattgtgtaaattgcctcgttgtaaatagacactcagtaaacattttcctcaccaaaatatttttaaggatttttctacccttctccttttctctttgctttccttttcttgcctgttctttccactccccccaaaatgatcagatagcaaatgtcttgataacatgaggtgccctcacattaaaaaacaaaatattgagccgggcgcggtggctcatgcctgtaatcccagcactttgggaggctgaggtgggcagatcgccttaggtcaggagttggagaccaggctgaccaatatgatgaaactctgtctctactaaaaattcaaaaatgtgccagacctggcctggtggcatgtgcctgtaatcccagctacttgggaggctgagtcataagcctgcaatgggaaaatggatcgaatctggggtgagggggaagtgatgtgggggttatggtacctcttttctcttccaaagatgctgttcttactgcatcacttgtggctggccaggaaaagccatgcaggagttttgtttgtggccactaggtgacgatcgtgttctgtacgggacctcttattaatagttcaccactagccgccactccagaagagcggaggaacccaggataatattttgtcaaccaagaaacaagaagtccctcccaggaactggaaatgaatggggaaaatgctgaaatctcatttgcactattcatttctcttctctctggaaagctcggcaatcatcaggtcatttcatttggcttaaattccatgtgtctttccaaacttttaaaagctggtgaaaattgttccacccatatgtaaaagaacataggttaagttgtctaattcttgcaggaatgtggatatagcattaaaaatatgtctttgtatacttatcttacccatgtaagaaaagagtggccaactttcatataaatagaaagagaacatttaagctatatgcagtttgcatttttgtctactattatgaaattattatctatgaaattcaagctgtaactcaacatatgtataattttaatttctaatttattgttagatctcagcacttaaaaaattacatcttgtatttgaattgttaaatctgttccctgcaaagaacagtaatacaatcatgttctaatttactagcatttgcatattttagaaatataatggcctgtaatttacttttcttttgcctataattttctgaagctctttatgatgcaccggtgcatttttatttaaaaaatagattgtgactcctcaaataatgttacaattcgatgttcaaaaagcaatccaggtacatagccataaagggatgagctagagaggtctccatattatcattcaatgtgagaataaaaattctatattttattctagaataaaattataaatttctttatcta

SEQ ID NO: 14 - protein, 337 aa, Homo sapiens
  Amino acid sequence of Homo sapiens G protein-coupled receptor 65 (GPR65)
MNSTCIEEQHDLDHYLFPIVYIFVIIVSIPANIGSLCVSFLQAKKESELGIYLFSLSLSDLLYALTLPLWIDYTWNKDNWTFSPALCKGSAFLMYMNFYSSTAFLTCIAVDRYLAVVYPLKFFFLRTRRFALMVSLSIWILETIFNAVMLWEDETVVEYCDAEKSNFTLCYDKYPLEKWQINLNLFRTCTGYAIPLVTILICNRKVYQAVRHNKATENKEKKRIIKLLVSITVTFVLCFTPFHVMLLIRCILEHAVNFEDHSNSGKRTYTMYRITVALTSLNCVADPILYCFVTETGRYDMWNILKFCTGRCNTSQRQRKRILSVSTKDTMELEVLE

SEQ ID NO: 15 - RNA, 2205 nt, Homo sapiens
  Nucleotide sequence encoding Homo sapiens Peptidase inhibitor 16 (CRISP9)
cctgggtgcaaccagtcacagctctgcagaggttactgtgattttgcccctgaaggatctgtccacaacttaggaactcacacagcttttggcctgagcccccgttaccaagagaaaggaggtttttgccaaggactccaaggggagtgcacttgatgctggtcgggacccaaagcgcccagccctccctgagacattgtgtgagtcgggctgggcctcaaacacggcccccactgccccaccccagccagggtggtgcttgtgtgggaaggactttaaatccagctgccagacccctggacgggagaaggagagacggctggccaccatgcacggctcctgcagtttcctgatgcttctgctgccgctactgctactgctggtggccaccacaggccccgttggagccctcacagatgaggagaaacgtttgatggtggagctgcacaacctctaccgggcccaggtatccccgacggcctcagacatgctgcacatgagatgggacgaggagctggccgccttcgccaaggcctacgcacggcagtgcgtgtggggccacaacaaggagcgcgggcgccgcggcgagaatctgttcgccatcacagacgagggcatggacgtgccgctggccatggaggagtggcaccacgagcgtgagcactacaacctcagcgccgccacctgcagcccaggccagatgtgcggccactacacgcaggtggtatgggccaagacagagaggatcggctgtggttcccacttctgtgagaagctccagggtgttgaggagaccaacatcgaattactggtgtgcaactatgagcctccggggaacgtgaaggggaaacggccctaccaggaggggactccgtgctcccaatgtccctctggctaccactgcaagaactccctctgtgaacccatcggaagcccggaagatgctcaggatttgccttacctggtaactgaggccccatccttccgggcgactgaagcatcagactctaggaaaatgggtactccttcttccctagcaacggggattccggctttcttggtaacagaggtctcaggctccctggcaaccaaggctctgcctgctgtggaaacccaggccccaacttccttagcaacgaaagacccgccctccatggcaacagaggctccaccttgcgtaacaactgaggtcccttccattttggcagctcacagcctgccctccttggatgaggagccagttaccttccccaaatcgacccatgttcctatcccaaaatcagcagacaaagtgacagacaaaacaaaagtgccctctaggagcccagagaactctctggaccccaagatgtccctgacaggggcaagggaactcctaccccatgcccaggaggaggctgaggctgaggctgagttgcctccttccagtgaggtcttggcctcagtttttccagcccaggacaagccaggtgagctgcaggccacactggaccacacggggcacacctcctccaagtccctgcccaatttccccaatacctctgccaccgctaatgccacgggtgggcgtgccctggctctgcagtcgtccttgccaggtgcagagggccctgacaagcctagcgtcgtgtcagggctgaactcgggccctggtcatgtgtggggccctctcctgggactactgctcctgcctcctctggtgttggctggaatcttctgaaggggataccactcaaagggtgaagaggtcagctgtcctcctgtcatcttccccaccctgtccccagcccctaaacaagatacttcttggttaaggccctccggaagggaaaggctacggggcatgtgcctcatcacaccatccatcctggaggcacaaggcctggctggctgcgagctcaggaggccgcctgaggactgcacaccgggcccacacctctcctgcccctccctcctgagtcctgggggtgggaggatttgagggagctcactgcctacctggcctggggctgtctgcccacacagcatgtgcgctctccctgagtgcctgtgtagctggggatggggattcctaggggcagatgaaggacaagccccactggagtggggttctttgagtgggggaggcagggacgagggaaggaaagtaactcctgactctccaataaaaacctgtccaacctgtggcaaaaaaaaaaaaaaaaa

SEQ ID NO: 16 - protein, 463 aa, Homo sapiens
  Amino acid sequence of Homo sapiens Peptidase inhibitor 16 (CRISP9)
MHGSCSFLMLLLPLLLLLVATTGPVGALTDEEKRLMVELHNLYRAQVSPTASDMLHMRWDEELAAFAKAYARQCVWGHNKERGRRGENLFAITDEGMDVPLAMEEWHHEREHYNLSAATCSPGQMCGHYTQVVWAKTERIGCGSHFCEKLQGVEETNIELLVCNYEPPGNVKGKRPYQEGTPCSQCPSGYHCKNSLCEPIGSPEDAQDLPYLVTEAPSFRATEASDSRKMGTPSSLATGIPAFLVTEVSGSLATKALPAVETQAPTSLATKDPPSMATEAPPCVTTEVPSILAAHSLPSLDEEPVTFPKSTHVPIPKSADKVTDKTKVPSRSPENSLDPKMSLTGARELLPHAQEEAEAEAELPPSSEVLASVFPAQDKPGELQATLDHTGHTSSKSLPNFPNTSATANATGGRALALQSSLPGAEGPDKPSVVSGLNSGPGHVWGPLLGLLLLPPLVLAGIF

SEQ ID NO: 17 - RNA, 1516 nt, Homo sapiens
  Nucleotide sequence encoding Homo sapiens Lysozyme (LYZ)
aaatactggggccagctcaccctggtcagcctagcactctgacctagcagtcaacatgaaggctctcattgttctggggcttgtcctcctttctgttacggtccagggcaaggtctttgaaaggtgtgagttggccagaactctgaaaagattgggaatggatggctacaggggaatcagcctagcaaactggatgtgtttggccaaatgggagagtggttacaacacacgagctacaaactacaatgctggagacagaagcactgattatgggatatttcagatcaatagccgctactggtgtaatgatggcaaaaccccaggagcagttaatgcctgtcatttatcctgcagtgctttgctgcaagataacatcgctgatgctgtagcttgtgcaaagagggttgtccgtgatccacaaggcattagagcatgggtggcatggagaaatcgttgtcaaaacagagatgtccgtcagtatgttcaaggttgtggagtgtaactccagaattttccttcttcagctcattttgtctctctcacattaagggagtaggaattaagtgaaaggtcacactaccattatttccccttcaaacaaataatatttttacagaagcaggagcaaaatatggcctttcttctaagagatataatgttcactaatgtggttattttacattaagcctacaacatttttcagtttgcaaatagaactaatactggtgaaaatttacctaaaaccttggttatcaaatacatctccagtacattccgttctttttttttttgagacagtctcgctctgtcgcccaggctggagtgcagtggcgcaatctcggctcactgcaacctccacctcccgggttcacgccattctcctgcctcagcctcccgagtagctgggattacgggcgcccgccaccacgcccggctaattttttgtatttttagtagagacagggtttcaccgtgttagccaggatggtctcgatctcctgaccttgtgatccacccacctcggcctcccaaagtgctgggattacaggcgtgagccactgcgcccggccacattcagttcttatcaaagaaataacccagacttaatcttgaatgatacgattatgcccaatattaagtaaaaaatataagaaaaggttatcttaaatagatcttaggcaaaataccagctgatgaaggcatctgatgccttcatctgttcagtcatctccaaaaacagtaaaaataaccactttttgttgggcaatatgaaatttttaaaggagtagaataccaaatgatagaaacagactgcctgaattgagaattttgatttcttaaagtgtgtttctttctaaattgctgttccttaatttgattaatttaattcatgtattatgattaaatctgaggcagatgagcttacaagtattgaaataattactaattaatcacaaatgtgaagttatgcatgatgtaaaaaatacaaacattctaattaaaggctttgcaacac

SEQ ID NO: 18 - protein, 148 aa, Homo sapiens
  Amino acid sequence of Homo sapiens Lysozyme (LYZ)
MKALIVLGLVLLSVTVQGKVFERCELARTLKRLGMDGYRGISLANWMCLAKWESGYNTRATNYNAGDRSTDYGIFQINSRYWCNDGKTPGAVNACHLSCSALLQDNIADAVACAKRVVRDPQGIRAWVAWRNRCQNRDVRQYVQGCGV

SEQ ID NO: 19 - RNA, 2151 nt, Homo sapiens
  Nucleotide sequence encoding Homo sapiens Vimentin (VIM)
gcctctccaaaggctgcagaagtttcttgctaacaaaaagtccgcacattcgagcaaagacaggctttagcgagttattaaaaacttaggggcgctcttgtcccccacagggcccgaccgcacacagcaaggcgatggcccagctgtaagttggtagcactgagaactagcagcgcgcgcggagcccgctgagacttgaatcaatctggtctaacggtttcccctaaaccgctaggagccctcaatcggcgggacagcagggcgcgtcctctgccactctcgctccgaggtccccgcgccagagacgcagccgcgctcccaccacccacacccaccgcgccctcgttcgcctcttctccgggagccagtccgcgccaccgccgccgcccaggccatcgccaccctccgcagccatgtccaccaggtccgtgtcctcgtcctcctaccgcaggatgttcggcggcccgggcaccgcgagccggccgagctccagccggagctacgtgactacgtccacccgcacctacagcctgggcagcgcgctgcgccccagcaccagccgcagcctctacgcctcgtccccgggcggcgtgtatgccacgcgctcctctgccgtgcgcctgcggagcagcgtgcccggggtgcggctcctgcaggactcggtggacttctcgctggccgacgccatcaacaccgagttcaagaacacccgcaccaacgagaaggtggagctgcaggagctgaatgaccgcttcgccaactacatcgacaaggtgcgcttcctggagcagcagaataagatcctgctggccgagctcgagcagctcaagggccaaggcaagtcgcgcctgggggacctctacgaggaggagatgcgggagctgcgccggcaggtggaccagctaaccaacgacaaagcccgcgtcgaggtggagcgcgacaacctggccgaggacatcatgcgcctccgggagaaattgcaggaggagatgcttcagagagaggaagccgaaaacaccctgcaatctttcagacaggatgttgacaatgcgtctctggcacgtcttgaccttgaacgcaaagtggaatctttgcaagaagagattgcctttttgaagaaactccacgaagaggaaatccaggagctgcaggctcagattcaggaacagcatgtccaaatcgatgtggatgtttccaagcctgacctcacggctgccctgcgtgacgtacgtcagcaatatgaaagtgtggctgccaagaacctgcaggaggcagaagaatggtacaaatccaagtttgctgacctctctgaggctgccaaccggaacaatgacgccctgcgccaggcaaagcaggagtccactgagtaccggagacaggtgcagtccctcacctgtgaagtggatgcccttaaaggaaccaatgagtccctggaacgccagatgcgtgaaatggaagagaactttgccgttgaagctgctaactaccaagacactattggccgcctgcaggatgagattcagaatatgaaggaggaaatggctcgtcaccttcgtgaataccaagacctgctcaatgttaagatggcccttgacattgagattgccacctacaggaagctgctggaaggcgaggagagcaggatttctctgcctcttccaaacttttcctccctgaacctgagggaaactaatctggattcactccctctggttgatacccactcaaaaaggacacttctgattaagacggttgaaactagagatggacaggttatcaacgaaacttctcagcatcacgatgaccttgaataaaaattgcacacactcagtgcagcaatatattaccagcaagaataaaaaagaaatccatatcttaaagaaacagctttcaagtgcctttctgcagtttttcaggagcgcaagatagatttggaataggaataagctctagttcttaacaaccgacactcctacaagatttagaaaaaagtttacaacataatctagtttacagaaaaatcttgtgctagaatactttttaaaaggtattttgaataccattaaaactgcttttttttttccagcaagtatccaaccaacttggttctgcttcaataaatctttggaaaaactcaaaaaaaaaaaaaaa

SEQ ID NO: 20 - protein, 466 aa, Homo sapiens
  Amino acid sequence of Homo sapiens Vimentin (VIM)
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTLLIKTVETRDGQVINETSQHHDDLE

SEQ ID NO: 21 - RNA, 7369 nt, Homo sapiens
  Nucleotide sequence encoding Homo sapiens Calmodulin regulated spectrin associated protein family member 2 (CAMSAP2)
acggggcggacctcgcgcggacggacggacggagacggcgccgccacattcctatgcccgggagcggcggcggcggcggcggcggctcccgcgggaggcggcaggcgcgcggcgcggacagctgagcttctcctccgtcggcgcccgggcggacatcgcccgggccccgatggtttgagcttgcttctccctccctcccgacccccgtggtggcgaggccacgccatgtgaaggttagggccgggacatcccgaggagccgcggtgaaagatgggggatgctgcagaccccagggagatgagaaagacgttcattgttccagccatcaagccttttgaccactatgatttctccagggccaaaatcgcctgcaatctggcctggctggtggccaaagcctttgggacagaaaatgtgccagaggaacttcaagaaccattttacacagatcagtatgaccaggaacacatcaaaccacctgttgttaatttgcttctatcggctgaactatactgtcgtgctgggagtctcattctcaagagtgatgctgcaaaaccccttttgggccatgatgctgtaatccaggctttagcacagaaaggtctttatgtcactgaccaggaaaaattggtaactgaacgagatctccacaagaaacccatacagatgagtgcacatttggccatgatcgataccctcatgatggcttatactgtagaaatggtcagtatagaaaaagtaattgcgtgtgctcagcagtattcagctttttttcaagccacagatctgccctatgatattgaggacgctgtcatgtactggataaataaggtaaatgaacatttgaaagacataatggaacaagaacaaaaactgaaagaacatcacacagttgaagctccaggaggtcaaaaggctcgttatcggaaagagcaaacattgcttaagcaactgccttgcattccattggtagaaaatttgttgaaggatgggacagatggctgtgcattagctgcccttattcatttttactgtcctgatgttgtcagattagaggatatttgtttgaaagaaactatgtctttggctgatagcctgtataatctgcagctgattcaagaattttgccaagaatacttgaaccagtgttgccatttcactctggaagatatgctctatgctgcttcatccataaagagtaattatttggtgttcatggcggaactgttctggtggtttgaagtggtgaagccgtcttttgtacagcctcgtgttgttcgtccacaaggagctgaacctgtaaaagatatgccttcaattcctgtcttgaatgctgccaaaagaaatgtcttagatagtagttctgacttcccttcaagtggggaaggagctacatttacacagtctcatcatcatttgccttctaggtattcacgtccccaggctcattcttcagcctcaggaggaattagaaggtcttcatctatgtcttatgttgatggcttcatagggacatggcccaaagagaaaagatcatcagtgcatggcgtatcatttgatatttcttttgataaagaagatagtgtacagagatccactccaaaccgaggaatcactcgttctattagtaatgaaggacttactctgaacaacagtcatgtatctaaacacattaggaaaaatttgtccttcaagccaataaatggagaagaggaagcagagagcattgaagaagaacttaatatagattctcacagtgacctcaaatcttgtgtgccccttaacacaaatgaactaaattctaatgagaatattcattacaagcttccaaatggagctttacaaaatagaatacttcttgacgagtttggcaatcagatcgagacaccaagcattgaagaagcattacaaataattcatgatactgaaaaatctcctcatacacctcagccagaccaaattgctaatggcttctttcttcatagtcaagaaatgagtatcttaaattcaaatatcaagttaaatcaatctagtcctgataatgtaactgatacgaaaggtgccttgagtcccataactgacaatactgaagtagacactggaattcacgttccttcagaagatattcctgaaactatggacgaagattcttcgttgagagattatactgtaagcttggactctgacatggatgatgcatctaaatttcttcaggattatgatattcgaactggcaacaccagggaagctttgagtccttgtccaagtactgtaagtaccaagtctcagccaggcagcagtgcttcttctagttctggagttaaaatgaccagctttgctgaacaaaaattcaggaaactgaatcataccgatggaaaaagtagtggaagcagttctcaaaaaactacaccagaaggctctgaacttaatattcctcatgtggttgcttgggcacaaattccagaagaaacagggcttccacagggacgggacactacccagctgttggcctctgaaatggtgcatcttaggatgaaactagaagaaaagaggcgtgctatagaagcccagaaaaagaaaatggaagctgcttttaccaaacagagacagaaaatgggaaggacagcattccttactgtagtgaaaaagaaaggggatgggatatctcctctacgagaggaagcggcgggtgcagaagatgagaaagtatatactgatcgagcaaaagaaaaggaatcacaaaaaactgatggacaaaggagcaagtcactggcagatataaaagagagcatggagaatcctcaagccaaatggctaaagtctccaactacacctattgatcctgagaagcagtggaacctggcaagcccctcagaagaaactttaaatgaaggagagattttagaatataccaaatccattgaaaagttaaattcatccctgcattttctacaacaagaaatgcaacgcttgtcacttcagcaggagatgttaatgcagatgagagagcaacaatcttgggtgatttcacctccacaaccctctccacagaaacagattcgagattttaagccttctaagcaggcaggcctgtcatcagccattgcaccattctcctcagactcccctcgtcctactcacccatctccacagtcttctaacaggaaaagtgcatctttttctgttaaaagtcaaaggactcctaggccaaatgagttaaaaataacacctttgaatcgaaccttgacacctcctcggtctgtggatagccttcctcggttaaggaggttttcaccaagtcaagttcctattcaaactaggtcatttgtatgttttggggatgatggagaacctcagttaaaggaatccaaacctaaagaggaagttaaaaaggaggaattggaatccaaagggactttggaacagcgtggacataatccagaagaaaaggaaatcaaaccttttgagtcaacagtctctgaagtcctatcactgcctgtcacagagactgtatgtctgacaccaaatgaggaccaattgaatcaacccacagaaccccctcctaaacccgttttcccacccactgctccaaaaaatgttaatctgattgaagtttccctctcagatttgaaaccccctgaaaaggctgatgtacctgttgaaaaatatgatggagaaagtgataaagaacaatttgatgatgaccagaaagtatgctgtggattcttttttaaggatgatcaaaaagcagaaaatgatatggcaatgaaacgggcagctttgttggagaaaagattaagaagggaaaaggaaactcagctccggaaacaacagttggaagcagaaatggagcataagaaggaggaaacaaggcgtaaaactgaggaagaacgtcagaagaaagaagatgagagagcacgcagagaatttattaggcaagaatatatgaggcggaaacaactgaaactaatggaagatatggatacagtaattaaaccccgtcctcaagtagtaaaacaaaaaaaacagcgaccaaaatctattcacagagatcatattgaatcccccaaaacaccaataaagggtcctccagtctctagcctttctttggcatcgctgaacacgggtgataacgagagtgtacattcaggcaagaggacgccaagatcagagtctgtagaaggcttcttatctccaagtcgttgtggcagtcgaaatggagaaaaagactgggagaatgcatcaacaacttcttcagtggcttctggaacagaatatacaggaccaaagctctacaaagaacccagtgcaaaatccaataagcacataatacaaaatgctttagctcattgctgtttggctggaaaagtaaatgaaggtcagaagaaaaaaatactggaggaaatggagaaatcagatgccaacaacttcttaatcttgttccgggattcaggatgccagttcagatctttatacacttattgcccagaaactgaagaaatcaataaactgactgggataggccctaaatctatcactaaaaaaatgattgaaggactttacaaatataattctgacaggaaacagtttagccacatacccgctaaaactttatctgccagtgttgatgcaattaccattcatagccatttatggcagaccaaaagaccagtaacacccaaaaaacttttacccactaaggcatagaagttgggaaatacttgcttcagaacattcatggtaaatttgcacttcatctttcctgcctatagaaaatctttctaattgccaacaagacttttattaattaaaactggacattaagctctgttgtcatgaacaactggaatgtaaaccacagtattttggagtgcagaacattctcaattaagtgataagtccaaatgatgaaggaaatgttttaattcacaaatggagatttgtatgtgttatcaggttcacctgcttgatattagatacattaaagcactgaattttcatggatattagttggatttatcattgaaatatggttaagattacaaattatgtgttttatttgttgcttttttttaaccttttaatgtatattcttgtcttcagatggtttgctatttttctctcctgggggtttattctaagatacctttgtattttatttcatgtggagatcatgaaagtaggaaatatacctttagaagtaactcgcacctttcttatgatgttaagagaaacactagtgtttagttttacagtaaccctcatattttaatggtgttacagcatttgcaaaaattattctgctaagtatttacaactctatttattattcactcaagtattaacattctctattaaataagaggaggtgttgtaaagagctgctagtaggttcgctttaaaccacatgagcttaaccaagaatatgttatgagaagttgctgattaaatcagtgctgtttttacaccacttctggccaactcagaataatttagattgttcttttaacaaaaaaggctttctatctcttttaaagtaagtcactttataagttggcagaagtgaatgacactttgagagtagtctttcaatctgaagatgtaagacttcctgaaacaagttctcaagaagtctttacattatatttataactcatataaaaattatatttagaatttttaaacatgtacaaagggctacattttaattttaaaatagcttcacattattttacttatattgggtttttcttcattttaatccttttcaagtggaatggcttagaataagtatacacttgaaatctcctctacatgatctttgttctttaacagtgtataccagagggttagttggggaaaaacttcattctcaggaaaagacttgaatgattatgtgaccctgttatatttcagtgttgtgacaaatgtgtaaactagcgggggaagacagtattgtatcataaatgagatgcgtagtttgttttctttcatgggaagtagagataaaaatatatacatttctctaattgagttgtttagagaaagaactaatgtctcatatgatgtatttacttattttaaaaaaaagaataggaatgagatgtccctgagctgtacttttctattattataaggcctttaggcatcagtgcatctgggttatcaacattttctcaaatgctgtcaatattttactgtaatttatgttcttatatttatgtatatttgttaaaactgtaaaaaaatttcacagatttttttccaatacctgtgcaagatacatgtgtagctcaaaactatttgtgatctactgtttgcatgtaagagaccaggatatgtaactcttatattttaagtgtatacatattgtgtatataacatatggatattaaaaatggggaattgcacattttaccttttggacagtaatttctatcacagttagaaggaaatgatagtcaaatacacgtttagattaaaactagtttaaaaaattataaatgaatctaatcaaaatgtgaatagtagtcaaaaggataatttaataagcattttacgttactaaatttgttcatttcaatattaactaaatttccctcatcaaagcaatctttgtgatattacttcgctattaaataaagaaaattggatgcaagacaatggagaaactttaaaactaaacaggaccaccctttattcttaaatttgtgtgtgtccaacagttgaattgaatgtctataaggtctaaaggtagaatgtgaatattgccacagagttcattgctctcagtataagattttactttattaatgcagaaggaatatggatatatttctttaagtctgcagatttttttattatggtgcagcttttttttaattatgtttttaaaattatacagttgaaaaatatgccatttcataaagtctgaggattttcgtcaaccttactgaaacacactggtgctttcatcatcagaggtcaaattattatgataactattccattaagtttgccaaacatttgtcgtggttaccagtgcagcctgtcaaattctgctatttgacacagctttggaaagatttagttcttggtttttccgttttgtattagaatgactgttacagttttatttggctgtttaaagccaaattcagctatttaattatggtttcatggacactgttgagcaatgtacagtgtatggtgtgcttacctgtccactctagagcattgcttacaggttttttgttttttaagatgctgtgctgtaaaatactgtcatacttgctatttcctggtacagtgtagtttttcccctttcatttgaataaaagcatggcaccaaatgaaaaaaaaaaaaaaa

SEQ ID NO: 22 - protein, 1489 aa, Homo sapiens
  Amino acid sequence of Homo sapiens Calmodulin regulated spectrin associated protein family member 2 (CAMSAP2)
MGDAADPREMRKTFIVPAIKPFDHYDFSRAKIACNLAWLVAKAFGTENVPEELQEPFYTDQYDQEHIKPPVVNLLLSAELYCRAGSLILKSDAAKPLLGHDAVIQALAQKGLYVTDQEKLVTERDLHKKPIQMSAHLAMIDTLMMAYTVEMVSIEKVIACAQQYSAFFQATDLPYDIEDAVMYWINKVNEHLKDIMEQEQKLKEHHTVEAPGGQKSPSKWFWKLVPARYRKEQTLLKQLPCIPLVENLLKDGTDGCALAALIHFYCPDVVRLEDICLKETMSLADSLYNLQLIQEFCQEYLNQCCHFTLEDMLYAASSIKSNYLVFMAELFWWFEVVKPSFVQPRVVRPQGAEPVKDMPSIPVLNAAKRNVLDSSSDFPSSGEGATFTQSHHHLPSRYSRPQAHSSASGGIRRSSSMSYVDGFIGTWPKEKRSSVHGVSFDISFDKEDSVQRSTPNRGITRSISNEGLTLNNSHVSKHIRKNLSFKPINGEEEAESIEEELNIDSHSDLKSCVPLNTNELNSNENIHYKLPNGALQNRILLDEFGNQIETPSIEEALQIIHDTEKSPHTPQPDQIANGFFLHSQEMSILNSNIKLNQSSPDNVTDTKGALSPITDNTEVDTGIHVPSEDIPETMDEDSSLRDYTVSLDSDMDDASKFLQDYDIRTGNTREALSPCPSTVSTKSQPGSSASSSSGVKMTSFAEQKFRKLNHTDGKSSGSSSQKTTPEGSELNIPHVVAWAQIPEETGLPQGRDTTQLLASEMVHLRMKLEEKRRAIEAQKKKMEAAFTKQRQKMGRTAFLTVVKKKGDGISPLREEAAGAEDEKVYTDRAKEKESQKTDGQRSKSLADIKESMENPQAKWLKSPTTPIDPEKQWNLASPSEETLNEGEILEYTKSIEKLNSSLHFLQQEMQRLSLQQEMLMQMREQQSWVISPPQPSPQKQIRDFKPSKQAGLSSAIAPFSSDSPRPTHPSPQSSNRKSASFSVKSQRTPRPNELKITPLNRTLTPPRSVDSLPRLRRFSPSQVPIQTRSFVCFGDDGEPQLKESKPKEEVKKEELESKGTLEQRGHNPEEKEIKPFESTVSEVLSLPVTETVCLTPNEDQLNQPTEPPPKPVFPPTAPKNVNLIEVSLSDLKPPEKADVPVEKYDGESDKEQFDDDQKVCCGFFFKDDQKAENDMAMKRAALLEKRLRREKETQLRKQQLEAEMEHKKEETRRKTEEERQKKEDERARREFIRQEYMRRKQLKLMEDMDTVIKPRPQVVKQKKQRPKSIHRDHIESPKTPIKGPPVSSLSLASLNTGDNESVHSGKRTPRSESVEGFLSPSRCGSRNGEKDWENASTTSSVASGTEYTGPKLYKEPSAKSNKHIIQNALAHCCLAGKVNEGQKKKILEEMEKSDANNFLILFRDSGCQFRSLYTYCPETEEINKLTGIGPKSITKKMIEGLYKYNSDRKQFSHIPAKTLSASVDAITIHSHLWQTKRPVTPKKLLPTKA

SEQ ID NO: 23 - RNA, 924 nt, Homo sapiens
  Nucleotide sequence encoding Homo sapiens Cathepsin G (CTSG)
gcacagcagcaactgactgggcagcctttcaggaaagatgcagccactcctgcttctgctggcctttctcctacccactggggctgaggcaggggagatcatcggaggccgggagagcaggccccactcccgcccctacatggcgtatcttcagatccagagtccagcaggtcagagcagatgtggagggttcctggtgcgagaagactttgtgctgacagcagctcattgctggggaagcaatataaatgtcaccctgggcgcccacaatatccagagacgggaaaacacccagcaacacatcactgcgcgcagagccatccgccaccctcaatataatcagcggaccatccagaatgacatcatgttattgcagctgagcagaagagtcagacggaatcgaaacgtgaacccagtggctctgcctagagcccaggagggactgagacccgggacgctgtgcactgtggccggctggggcagggtcagcatgaggaggggaacagatacactccgagaggtgcagctgagagtgcagagggataggcagtgcctccgcatcttcggttcctacgacccccgaaggcagatttgtgtgggggaccggcgggaacggaaggctgccttcaagggggattccggaggccccctgctgtgtaacaatgtggcccacggcatcgtctcctatggaaagtcgtcaggggttcctccagaagtcttcaccagggtctcaagtttcctgccctggataaggacaacaatgagaagcttcaaactgctggatcagatggagacccccctgtgactgactcttcttctcggggacacaggccagctccacagtgttgccagagccttaataaacgtccacagagtataaataaccaattcctcatttgttcattaaacgtcattcagtactta

SEQ ID NO: 24 - protein, 255 aa, Homo sapiens
  Amino acid sequence of Homo sapiens Cathepsin G (CTSG)
MQPLLLLLAFLLPTGAEAGEIIGGRESRPHSRPYMAYLQIQSPAGQSRCGGFLVREDFVLTAAHCWGSNINVTLGAHNIQRRENTQQHITARRAIRHPQYNQRTIQNDIMLLQLSRRVRRNRNVNPVALPRAQEGLRPGTLCTVAGWGRVSMRRGTDTLREVQLRVQRDRQCLRIFGSYDPRRQICVGDRRERKAAFKGDSGGPLLCNNVAHGIVSYGKSSGVPPEVFTRVSSFLPWIRTTMRSFKLLDQMETPL

SEQ ID NO: 25 - RNA, 1421 nt, Homo sapiens
  Nucleotide sequence encoding Homo sapiens Glyceraldehyde-3-phosphate dehydrogenase (GAPDH)
gcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccagttaaaaaaaaaaaaaaaaaaaaa

SEQ ID NO: 26 - protein, 335 aa, Homo sapiens
  Amino acid sequence of Homo sapiens Glyceraldehyde-3-phosphate dehydrogenase (GAPDH)
MGKVKVGVNGFGRIGRLVTRAAFNSGKVDIVAINDPFIDLNYMVYMFQYDSTHGKFHGTVKAENGKLVINGNPITIFQERDPSKIKWGDAGAEYVVESTGVFTTMEKAGAHLQGGAKRVIISAPSADAPMFVMGVNHEKYDNSLKIISNASCTTNCLAPLAKVIHDNFGIVEGLMTTVHAITATQKTVDGPSGKLWRDGRGALQNIIPASTGAAKAVGKVIPELNGKLTGMAFRVPTANVSVVDLTCRLEKPAKYDDIKKVVKQASEGPLKGILGYTEHQVVSSDFNSDTHSSTFDAGAGIALNDHFVKLISWYDNEFGYSNRVVDLMAHMASKE

SEQ ID NO: 27 - RNA, 1435 nt, Homo sapiens
  Nucleotide sequence encoding Homo sapiens Hypoxanthine phosphoribosyl transferase 1 (HPRT1)
ggcggggcctgcttctcctcagcttcaggcggctgcgacgagccctcaggcgaacctctcggctttcccgcgcggcgccgcctcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaaaaaaaaaaaaaaaaaaaaaa

SEQ ID NO: 28 - protein, 218 aa, Homo sapiens
  Amino acid sequence of Homo sapiens Hypoxanthine phosphoribosyl transferase 1 (HPRT1)
MATRSPGVVISDDEPGYDLDLFCIPNHYAEDLERVFIPHGLIMDRTERLARDVMKEMGGHHIVALCVLKGGYKFFADLLDYIKALNRNSDRSIPMTVDFIRLKSYCNDQSTGDIKVIGGDDLSTLTGKNVLIVEDIIDTGKTMQTLLSLVRQYNPKMVKVASLLVKRTPRSVGYKPDFVGFEIPDKFVVGYALDYNEYFRDLNHVCVISETGKAKYKA